SEQUENCE LISTING (3 sequences)

SEQ ID NO: 1 - DNA, 20 bp, Artificial Sequence
  Description of Artificial Sequence  Synthetic Primer
cctgggcgcagtcaatgtgg

SEQ ID NO: 2 - DNA, 20 bp, Artificial Sequence
  Description of Artificial Sequence  Synthetic Primer
ggtgatagctgacgcggtcc

SEQ ID NO: 3 - DNA, 22 bp, Artificial Sequence
  Description of Artificial Sequence  Synthetic Primer
agttgaggggactttcccaggc

(19) United States
(12) Patent Application Publication
(10) Pub. No.: US 2004/0047919 A1
(43) Pub. Date: Mar. 11, 2004

(54) Methods and compositions involving aldose reductase inhibitors

(75) Inventors: Satish K. Srivastava, Galveston, TX (US); K. Venkat Ramana, Galveston, TX (US); Aruni Bhatnagar, Louisville, KY (US)

Correspondence Address: Gina N. Shishima, Fulbright & Jaworski L.L.P., Suite 2400, 600 Congress Avenue, Austin, TX, 78701, US

(73) Assignee: Board of Regents, The University of Texas System

(21) Appl. No.: 10/462,223

(22) Filed: Jun. 13, 2003

Related U.S. Application Data
(60) Provisional application No. 60/388,213, filed on Jun. 13, 2002.

(51) Int. Cl. A61K 33/00

(52) U.S. Cl. 424/718

(57) ABSTRACT

Embodiments of the invention include methods and compositions for the inhibition of Aldose Reductase by nitric oxide. Certain embodiments of the invention include the induction of nitric oxide by administration of a nitric oxide donor, nitric oxide precursor, inhibitor of a nitric oxide synthase inhibitor, and/or an activator of nitric oxide synthase. Methods may include the treatment of various disease states by inhibiting Aldose Reductase.

This application claims priority to U.S. Provisional Patent Application No. 60/388,213 filed on Jun. 13, 2002, which is hereby incorporated by reference in its entirety.

The government may own rights in the present invention pursuant to grant number from the National Institutes of Health, grant numbers DK36118, HL55477, EY01677, and HL59378.

### BACKGROUND OF THE INVENTION

### DESCRIPTION OF RELATED ART

Aldose reductase (AR) catalyzes the reduction of a wide range of aldehydes (Bhatnager and Srivastava, 1992). The substrates of the enzyme range from aromatic and aliphatic aldehydes to aldoses such as glucose, galactose, and ribose. The reduction of glucose by AR is particularly significant during hyperglycemia and increased flux of glucose via AR has been etiologically linked to the development of secondary diabetic complications (Bhatnager and Srivastava, 1992; Yabe-Nishimura, 1998). However, recent studies showing that AR is an excellent catalyst for the reduction of lipid peroxidation-derived aldehydes and their glutathione conjugates (Srivastava et al., 1995; Vander Jagt et al., 1995; Srivastava et al., 1998; Srivastava et al., 1999; Dixit et al., 2000; Ramana et al., 2000) suggest that in contrast to its injurious role during diabetes, under normal glucose concentration, AR may be involved in protection against oxidative and electrophilic stress. The antioxidant role of AR is consistent with the observations that in a variety of cell types AR is upregulated by oxidants such as hydrogen peroxide (Spycher et al., 1997), lipid peroxidation-derived aldehydes (Ruef et al., 2000; Rittner et al., 1999), advanced glcosylation end products (Nakamura et al., 2000) and nitric oxide (Seo et al., 2000). The expression of the enzyme is also increased under several pathological conditions associated with increased oxidative or electrophilic stress such as iron overload (Barisani et al., 2000), alcoholic liver disease (O'Connor et al., 1999), heart failure (Yang et al., 2000), myocardial ischemia (Shinmura et al., 2000), vascular inflammation (Rittner et al., 1999) and restenosis (Ruef et al., 2000).

Although glucose is a poor substrate of AR, the enzyme is recruited in renal tissues to generate sorbitol for balancing the osmotic gap during diureseis (Burg et al., 1997). The abundance and the transcription of the AR gene are dramatically enhanced by the activation of the transcription factor-TonE-binding protein (Miyakawa et al., 1999; Ko et al., 2000). However, osmotic role of AR in non-renal tissues is unclear, and the high expression of the enzyme in tissues such as heart, blood vessels, skeletal muscle or brain suggests that the enzyme may be involved in processes other than osmoregulation and glucose metabolism. Recent evidence shows that in addition to osmotic or oxidative stress, AR and its homologs are also upregulated by mitogenic stimuli. Stimulation of NIH 3T3 cells by FGF-1 (and to a lesser extent by FGF-2, EGF and phorbol esters) leads to a dramatic increase in the expression of an aldo-keto reductase-FR-1, (Donohue et al., 1994) which is related to AR in structure and function (Donohue et al., 1994; Srivastava et al., 1998). The AR protein itself is also increased by growth factors in the 3T3 fibroblasts (Hsu et al., 1997), astrocytes (Jacquin-Becker and Labourdette, 1997) and the vascular smooth muscle cells (VSMC; Ruef et al., 2000). Although the quiescent VSMC of the tunica media do not express detectable levels of AR, the expression of the enzyme is markedly induced during vascular inflammation or growth (Ruef et al., 2000; Rittner et al., 1999). Moreover, The inventors have previously shown that inhibition of AR prevents serum-induced VSMC growth in culture and neointima formation in balloon-injured rat carotid arteries (Ruef et al., 2000).

Extensive investigations show that diabetes is associated with the impairment of NO-mediated vascular relaxation and a decrease in NO bioavailability, which may be a causative factor in other complications as well (Kassab et al., 2001). The second messenger NO is a diffusible gas that regulates several physiological processes, including blood pressure, platelet aggregation, and neurotransmission (van Goor et al., 2001; Torreilles, 2001; West et al., 2002). In addition, recent studies show that NO regulates glucose and oxygen consumption in the heart (Traverse et al., 2002; Recchia, 2002). However, previous studies have shown that incubation of VSMC with NO-donors results in the transcriptional upregulation of AR (Seo et al., 2000). Thus, the mechanisms of vascular injury are not well understood.

Furthermore, the role of aldose reductase in a number of diseases and conditions requires elucidation, as patients with these diseases and conditions continue to require new treatments. Thus, there is a need for screening and therapeutic methods involving aldose reductase.

### SUMMARY OF THE INVENTION

The present invention concerns the discovery that aldose reductase (AR) can be modified to affect its activity. More particularly, it was discovered that nitric oxide regulates the polyol pathway of glucose metabolism (through aldose reductase) and that aldose reductase mediates mitogenic signaling. Thus, the present invention concerns preventative, prognostic, and therapeutic compositions and methods that affect or are implicated in glucose metabolism and/or mitogenic signaling. Moreover, the present invention concerns the discovery that aldose reductase is also involved in apoptotic pathways, particularly those in which TNF-α plays a role. Thus, the present invention concerns preventative, prognostic, and therapeutic compositions and methods that affect or are implicated in apoptosis, particularly apoptosis of vascular endothelial cells and vascular smooth muscle cells. Additionally, the present invention concerns the discovery that inhibition of AR leads to inhibition or downregulation of NF-κB activity, particularly NF-κB activity that has been induced by TNF-α. Consequently, the present invention concerns preventative, prognostic, and therapeutic compositions and methods that affect or are implicated in NF-κB activity or TNF-α activity. Also, the present invention concerns the discovery that S-glutathiolation of AR can inhibit its activity. Therefore, the present invention concerns screening methods and compositions involving assaying for S-glutathiolation of AR, as well as preventative, prognostic, and therapeutic compositions and methods that affect or are implicated in S-glutathiolation of AR.

It is contemplated that activity of an enzyme or polypeptide can be affected directly or indirectly, and can include, but is not limited to, modifying or modulating, altering, reducing, down-regulating, inhibiting, eliminating, increasing, enhancing, inducing, up-regulating transcription, translation, post-translation modification, binding activity, enzyme activity, stability, localization, protein conformation, protein-protein interactions, signalling, or co-factor interaction. The term “inhibitor” in the context of a polypeptide, such as AR inhibitor, refers to a substance or compound that directly or indirectly inhibits (decrease, limit, or block—according to its ordinary and plain meaning) the activity of the polypeptide in a given context. Similarly, the term “inducer” in the context of a polypeptide refers to a substance or compound that directly or indirectly induces (initiate or increase—according to its ordinary and plain meaning) the activity of the polypeptide in a given context.

The present invention concerns methods of reducing, inhibiting, affecting and/or generally modulating aldose reductase activity in a cell. Methods of the invention further include, but are not limited to, methods of reducing the risk of diabetes complications; methods of reducing the risk of diabetes complications in a patient; methods for preventing or treating inflammation in a cell or patient; methods for reducing an immune response in a patient; methods for preventing or treating allergies; methods for treating or preventing anaphylaxis; methods for relieving, treating, or preventing asthma symptoms; methods for reducing a reaction to a toxin; methods for preventing or treating hyperglycemia-induced atherosclerosis (may include with stent in); methods for preventing or treating restenosis; methods of reducing or preventing stress-induced change in a cell or patient; methods of treating or preventing cancer; methods of inhibiting apoptosis; methods of inhibiting NF-κB activity; methods of inhibiting TNF-α; and methods of reducing ICAM-1 activity. In methods of the invention, embodiments involve administering or providing to a cell or patient an effective amount of a composition comprising an AR inhibitor, particularly, as in some embodiments, a nitric oxide inducer. An effective amount refers to the amount that accomplishes a particular goal. In some embodiments an effective amount results in a therapeutic benefit, which is understood to encompass any therapeutic benefit to the cell or patient.

A list of nonexhaustive examples of such benefit includes extension of the subject's life by any period of time, decrease or delay in the progression of the disease, and alleviation of one or more symptoms that can be attributed to the subject's condition or disease.

In some embodiments of the invention, a nitric oxide inducer is provided or administered to the cell to modulate an aldose reductase polypeptide in a cell. In particular embodiments, the inducer inhibits AR. It is contemplated in some embodiments that aldose reductase is modulated by chemically modifying the cysteine located at position 298 in a aldose reductase polypeptide or the corresponding cysteine (which may be at a different position, depending on organism) of the aldose reductase in the cell. It is contemplated that the present invention is not limited to any particular aldose reductase disclosed in the Examples, but can be extended to any aldose reductase polypeptide recognized in the art, particularly other mammalian AR polypeptides. The methods and compositions of the invention are all contemplated for use in mammalian cells and organisms, particularly humans.

A nitric oxide inducer (NO inducer) refers to any compound that increases the amount of available nitric oxide. A nitric oxide inducer includes, but is not limited to, nitric oxide precursors, nitric oxide donors, or inhibitors of nitric oxide synthase inhibitor. Furthermore, nitric oxide donors include nitric oxide synthase substrates. In some embodiments of the invention, a nitric oxide precursor is the NO inducer. In still further embodiments, the precursor is L-arginine. In other embodiments of the invention, the nitric oxide inducer is a nitric oxide donor. The nitric oxide donors include nitric oxide synthase substrates, sildenafil citrate, or nitroglycerine in any form. In some embodiments, the nitroglycerine is provide to the patient as a patch. Nitric oxide synthase substrates include L-arginine. In still further embodiments, a nitric oxide inducer is an inhibitor of a nitric oxide synthase inhibitor or an activator of nitric oxide synthase. In some embodiments, the nitric oxide inducer inhibits at least one of the following nitric oxide synthase inhibitors: L-NAME and L-NNA.

Other AR inhibitors of the invention include 4-hydroxy-trans-2-nonenal (HNE) and glutathione disulfide (GSSG).

It is contemplated that the compositions of the invention may comprise more than one nitric oxide inducer, and could involve 1, 2, 3, 4, 5 or more such inducers, administered simultaneously or sequentially.

In some embodiments, the diabetes complication is cataractogenesis, neuropathy, nephropathy, retinopathy, vasculopathy, atherosclerosis, restenosis, artery or vein graft rejection, or wound healing.

Methods of the invention may include further steps. In some embodiments, a patient with the relevant condition or disease is identified or a patient at risk for the condition or disease is identified. A patient may be someone who has not been diagnosed with the disease or condition (diagnosis, prognosis, and/or staging) or someone diagnosed with disease or condition (diagnosis, prognosis, monitoring, and/or staging), including someone treated for the disease or condition (prognosis, staging, and/or monitoring). Alternatively, the person may not have been diagnosed with the disease or condition but suspected of having the disease or condition based either on patient history or family history, or the exhibition or observation of characteristic symptoms.

Methods of the invention involve patients, or the cells of patients, who have, exhibit signs or symptoms of, or at risk for diabetes, diabetes complications, toxic shock, allergy, asthma, anaphylaxis, hyperglycemia-induced atherosclerosis, cataractogenesis, neuropathy, nephropathy, retinopathy, vasculopathy, an open wound, inflammation, restenosis, artery or vein graft rejection, complications from or with wound healing, microvaculopathy, macroangiopathy, heart disease, stroke, ischemia, septicemia, ischemic damage, arteriosclerosis, iron overload, alcholic liver disease, hear failure, myocardial ischmia, vascular inflammation, or stress. It is specifically contemplated that methods discussed with respect to a particular disease, condition, or symptom, may be implemented with respect to other diseases, conditions, or conditions discussed herein.

Further step that may be included are providing to the patient or cells other therapeutics or preventative agents. Examples include insulin, epinephrine or adrenalin derivatives or analogs, chemotherapeutics, radiotherapeutics or other anti-cancer agents (gene therapy, immunotherapy, surgery—tumor resection), anti-inflammatory agents, and medicine or therapy for the treatment of restenosis, atherosclerosis, cataractogenesis, neuropathy, nephropathy, retinopathy, vasculopathy, atherosclerosis, restenosis, artery or vein graft rejection, or wound healing.

In some embodiments of the invention, as part of a therapeutic treatment, patients are administered an NF-κB inhibitor, such as IκB-α, or nucleic acid molecules with a site to which NF-κB binds, an anti-NF-κB antibody, an NF-κB ribozyme or siRNA, or an IκB inducer.

Compositions may be administered to the cell or patient directly, locally, topically, orally, endoscopically, intratracheally, intratumorally, intravenously, intralesionally, intramuscularly, intraperitoneally, regionally, percutaneously, or subcutaneously. Compositions, in some embodiments are in a pharmaceutically acceptable formulation.

Other compositions of the invention to effect modulation of aldose reductase involve a nitric oxide inducer, a hydrogen peroxide inducer, lipid-peroxidation derived aldehydes, and/or advanced glycosylation end products.

In some embodiments of the invention, compositions concern inhibitors of aldose reductase. Such inhibitors may include nucleic acid compositions. In further embodiments, the compositions are antisense, ribozyme, and siRNA that inhibit aldose reductase.

Methods of the invention also include screening methods to identify candidate therapeutic compounds, particularly those that generally have an AR-inhibitory effect. Methods of screening include assaying candidate compounds that effect a reduction, elimination, or inhibition of NF-κB or TNF-α activity. In addition to directly affecting the activity of either protein, the candidate compound may indirectly affect activity by altering expression, stability, localization or processing of the protein. In some embodiments, the activity of NF-κB is reduced by reducing the amount of NF-κB capable of activating transcription.

Candidate compounds include but are not limited to nucleic acids, such as DNA, RNA, oligonucleotides, antisense molecules, ribozymes, siRNA, nucleotide analogs, aptamers; proteinaceous compositions, such as peptides, polypeptides, proteins, antibodies, peptide mimetics, peptide nucleic acids, amino acid analogs; fusion proteins, chimeric proteins; and, small molecules, such as inorganic and organic small molecules.

In specific embodiments, there are methods of screening for a candidate aldose reductase inhibitor comprising: a) contacting aldose reductase with a candidate substance; and, b) assaying for S-glutathiolation of aldose reductase, wherein S-glutathiolation of aldose reductase identifies substance as a candidate aldose reductase inhibitor. In some embodiments, the invention also includes assaying the activity of S-glutathiolated aldose reductase. In other embodiments, the candidate substance is an NO donor.

Another screening method of the invention includes a method of screening for an aldose reductase inhibitor comprising: a) stimulating a cell with TNF-α in the presence of a candidate substance, b) assaying for apoptosis of the cell, wherein inhibition of apoptosis identifies the cell as a candidate aldose reductase inhibitor; and, c) determining whether the candidate aldose reductase inhibitor inhibits the activity of aldose reductase.

The present invention also concerns methods of reducing ICAM-1 expression in a cell comprising administering to the cell an effective amount of a composition comprising an aldose reductase inhibitor. Other aspects of the invention include methods of inhibiting TNF-α-induced apoptosis in a cell comprising administering to the cell an effective amount of a composition comprising an aldose reductase inhibitor. In still further aspects there are methods of inhibiting apoptosis of a vascular endothelial cell comprising administering to the cell an effective amount of a composition comprising an aldose reductase inhibitor.

In some cases, cells of the invention are in a patient exhibiting symptoms of atherosclerosis, restenosis, microvaculopathy, or macroangiopathy or the patient is at risk for atherosclerosis, restenosis, microvaculopathy, or macroangiopathy.

It is specifically contemplated that any limitation discussed with respect to one embodiment of the invention may apply to any other embodiment of the invention. Furthermore, any composition of the invention may be used in any method of the invention, and any method of the invention may be used to produce or to utilize any composition of the invention.

The use of the term “or” in the claims is used to mean “and/or” unless explicitly indicated to refer to alternatives only or the alternative are mutually exclusive, although the disclosure supports a definition that refers to only alternatives and “and/or.”

Throughout this application, the term “about” is used to indicate that a value includes the standard deviation of error for the device or method being employed to determine the value.

As used herein the specification, “a” or “an” may mean one or more, unless clearly indicated otherwise. As used herein in the claim(s), when used in conjunction with the word “comprising,” the words “a” or “an” may mean one or more than one. As used herein “another” may mean at least a second or more.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### DESCRIPTION OF THE FIGURES

FIG. 1: Inhibition of AR prevents NF-κB activation in balloon-injured arteries. Cross sections of balloon-injured arteries were obtained from uninjured rat carotid arteries and after 10 days of injury from rat that were treated with the vehicle or 10 mg/kg/day tolrestat and were stained with antibodies directed against activated NF-κB. Immunoreactivity of the antibodies is evident as a dark brown stain, whereas the non-reactive areas display only the background color. The extent of immunoreactivity was quantified by image analysis and is shown in Panel D. The bars represent mean immunoreactivity in the neointima of 5 animals±SEM. *P<0.05 compared to control (untreated) rats.

FIG. 2A, FIG. 2B, FIG. 2C, FIG. 2D and FIG. 2E: Inhibition of AR prevents TNF-α-induced proliferation. Growth-arrested rat VSMC were stimulated with the indicated concentrations of either TNF-α or sorbinil for 24 h. Cell proliferation was determined by measuring the incorporation of [³H]-thymidine (10 μCi/ml), added 6 h prior to the end of the experiment. The extent of proliferation is expressed a percent increase compared to serum-starved cells stimulated with the vehicle alone. FIG. 2A The dependence of VSMC proliferation on TNF-α concentration in the absence and the presence of 10 μM sorbinil. FIG. 2B Inhibition VSMC growth by different concentration sorbinil in the absence and the presence of 2 nM TNF-α. To examine the effect of AR inhibitors the VSMC were incubated with 10 μM sorbinil or tolrestat for 24 h without or with 2 nM TNF-α and the number of cells FIG. 2C, MTT reactivity FIG. 2D and FIG. 2E [³H]-thymidine incorporation were measured as described in the text. Control dishes were stimulated with the vehicle alone. Bars represent mean±SEM (n=4), *P<0.05, **P<0.01 compared with treatment without the inhibitor.

FIG. 3A, FIG. 3B and FIG. 3C: AR inhibitors attenuate TNF-α-induced VSMC proliferation. Quiescent VSMC were either left untreated or were pre-incubated with the AR inhibitors, sorbinil and tolrestat (10 μM each) and were then exposed to TNF-α (2 nM) for 24 h. The VSMC proliferation was determined by the addition of [³H]-thymidine (10 μCi/ml) 6 h prior to completion of incubation period, or by MTT assay and counting the number of cells as described under Materials and Methods. Bar graphs represent fold change in the cell growth as determined by FIG. 3A; Cell count, FIG. 3B; MTT assay and FIG. 3C; [³H]-thymidine incorporation.

FIG. 4A and FIG. 4B: Attenuation of TNF-α-induced VSMC proliferation by ARI is not due to apoptosis. Quiescent VSMC without and after pretreatment with AR inhibitors, sorbinil and tolrestat (10 μM each), were exposed to TNF-α (2 nM) for 24 h and then the VSMC apoptosis FIG. 4A and caspase-3 activation FIG. 4B were determined by using Rochie's cell death ELISA detection kit and using caspase-3 specific substrate, Z-DEVD-AFC.

FIG. 5A and FIG. 5B: Inhibition of AR abrogates PKC activation. FIG. 5A Quiescent VSMC were preincubated with 10 μM sorbinil or tolrestat for 24 h, FIG. 5B the VSMC were transiently transfected with AR antisense or scrambled control oligonucleotide as described in the experimental procedures, subsequently the cells were stimulated with TNF-α (0.1 nM), bFGF (5 ng/ml), PDGF-AB (5 ng/ml), Ang-II (2 μM) or PMA (10 nM) for 4 h and the membrane-bound PKC activity was determined as described in the text. In FIG. 5A Bars represent mean±SEM (n=4). **P<0.01, ***P<0.001 and ^{##} non significant, compared with the activity without the inhibitor. In FIG. 5B Bars represent mean±SEM (n=4). *P<0.01, **P<0.001 compared with the activity in the Scrambled control oligonucleotide transfected cells. The inset in B shows the AR expression as determined by Western blot analysis in VSMC transfected with antisense AR.

FIG. 6A and FIG. 6B: Transient transfection of antisense AR prevents TNF-α-induced proliferation of VSMC. Quiescent VSMC were either left untreated or preincubated with AR antisense or srambled oligonucleotides as described in the text. After 24 h of treatment, the cells were stimulated with 2 nM TNF-α or medium and the number of cells FIG. 6A and MTT reactivity FIG. 6B were measured. Bars represent mean±SEM (n=4).

FIG. 7: AR inhibitors attenuate TNF-α-induced membrane bound PKC activation in VSMC. Quiescent VSMC were preincubated with 10 μM of sorbinil or tolrestat for 24 h. Subsequently the cells were stimulated with 2 nM of TNF-α for 4 h at 37° C. The cytosolic and membrane bound fractions were separated as described in the text. The activation of PKC was assayed by using Promega SignaTECT PKC assay system.

FIG. 8A: Regulation of aldose reductase activity and sorbitol content in the aorta by NO. The abdominal aortas of Sprague-Dawley rats, C57/BL6 mice and eNOS-null mice in the C57/BL6 background were dissected into rings and incubated with 2 mM L-arginine or 1 mM L-NAME for 12 h and then glucose was added to a final concentration of 50 mM. After 24 h, the pieces of aorta were homogenized and their AR activity and sorbitol content measured as described in the experimental procedures. Error bars represent S.D. of mean for 3 separate experiments. ^{**}P<0.001, ^{*}<0.01 and ^{#} non-significant compared to the C57/BL6 mice.

FIG. 8B: Reversible inactivation of aldose reductase by NO. The VSMC were incubated in KH buffer containing 1 mM SNAP for 0-2 h and AR activity was determined as described in Materials and Methods. To examine regeneration of AR activity, the cells were washed with KH buffer and reincubated in fresh media without SNAP for 4 to 12 h. AR activity in VSMC was determined at the different time periods.

FIG. 9A and FIG. 9B: In vitro modification of AR by NO donors. Purified human recombinant AR was reduced with 100 mM DTT and passed through PD10 column to remove excess of DTT. The reduced enzyme was incubated with nitrogen saturated 100 mM potassium phosphate buffer (pH 7.0) containing 1 mM EDTA with indicated concentrations of either freshly prepared GSNO (FIG. 9A) or glyco-SNAP (FIG. 9B) at room temperature. AR activity was determined at different time intervals by using DL-glyceraldehyde as substrate as described in the examples.

FIG. 10A and FIG. 10B: ESI-MS of GSNO or glyco-SNAP modified recombinant AR. The reduced enzyme was incubated with GSNO (FIG. 10A) and glyco-SNAP (FIG. 10B) in 0.1 M potassium phosphate buffer (pH 7.0) for 60 min and 10 min, respectively. Excess of NO donors was removed by passing through PD10 column and the ESI-MS of the desalted mixture was determined as described in Example 3.

FIG. 11: Inhibition of AR attenuates TNF-α-induced changes in cell growth. Quiescent VEC without and with pretreatment with AR inhibitors, sorbinil and tolrestat (10 μM), were exposed to TNF-α (2 nM) for 24 h and the VEC proliferation was determined by the addition of [³H]-thymidine (10 μCi/ml) 6 h prior to completion of incubation period as described in the examples.

FIG. 12A and FIG. 12B: Inhibition of AR attenuates TNF-α-induced apoptosis. Quiescent VEC without and with pretreatment with AR inhibitors, sorbinil and tolrestat (10 μM), were exposed to TNF-α (2 nM) for 24 h. Apoptosis of VEC was measured by nucleosomal degradation by using Rochie's cell death ELISA detection kit (FIG. 12A) and caspase-3 activation by using caspase-3 specific substrate, Z-DEVD-AFC (FIG. 12B) as described in the examples.

FIG. 13A, FIG. 13B and FIG. 13C: Inhibition of AR prevents antiproliferative effects of high glucose and TNF-α in HLEC. Growth-arrested HLEC were stimulated with either 50 mM glucose (high glucose) or 2 nM TNF-α in the absence and presence of AR-inhibitors, sorbinil or tolrestat (10 μM). After 24 h, cell growth and viability were determined by counting the number of cells in the dish (FIG. 13A), MTT assay (FIG. 13B) and the incorporation of [³H]-thymidine added 6 h prior to the end of the experiment (FIG. 13C). Columns represent mean±SE (n=4); **P<0.01 compared with serum-starved cells untreated with either TNF-α or high glucose.

FIG. 14A and FIG. 14B: Inhibition of AR prevents high glucose and TNF-α-induced apoptosis and the activation of caspase-3. Growth-arrested HLEC were stimulated with either 50 mM glucose (high glucose) or 2 nM TNF-α in the absence and presence of AR-inhibitors, sorbinil or tolrestat (10 μM) for 24 h. FIG. 14A Apoptosis was evaluated by using “Cell Death Detection ELISA” kit (Roche Inc.) that measures cytoplasmic DNA-histone complexes, generated during apoptotic DNA fragmentation. The cell death detection was performed according to the manufacture's instructions and monitored spectrophotometrically at 405 nm. FIG. 14B Caspase-3 activation was measured by increase in fluorescence (excitation: 400 nm; emission: 505 nm) due to cleavage of substrate (Z-DEVD-AFC, CBZ-Asp-Glu-Val-Asp-AFC). Columns represent mean±SE (n=4), *P<0.01, **P<0.01 compared with cells left untreated with either high glucose or TNF-α.

FIG. 15A, FIG. 15B, FIG. 15C and FIG. 15D: Inhibition of AR prevents phosphorylation and degradation of IκB-α. Quiescent HLEC were left either untreated (left panel) or pre-incubated with 10 or 20 μM sorbinil for 24 h, and then stimulated with glucose 50 mM or 0.1 nM TNF-α (right panels). After the indicated duration of exposure, the cells were harvested, lysed and cytosolic extracts were prepared as described in the text. The cytosolic extracts were separated by SDS-PAGE by loading equal amounts of protein in each lane. Western blots were developed using antibodies directed against phospho-IκB-α protein FIG. 15A and FIG. 15C or unphosphorylated IκB-α FIG. 15B and FIG. 15D to determine the total IκB-α protein.

FIG. 16A and FIG. 16B: Inhibition of AR abrogates PKC activation. FIG. 16A Quiescent HLEC were incubated with 10 μM sorbinil or tolrestat for 24 h, FIG. 16B the HLEC were transiently transfected with AR antisense or scrambled control oligonucleotides. Subsequently, the cells were stimulated with high glucose (50 mM), TNF-α (0.1 nM) or PMA (10 nM) for 4 h and the membrane-bound PKC activity was determined as described in the text. The bars represent mean±SE (n=4). **P<0.001, compared with the activity without the inhibitor FIG. 16A or with the scrambled control oligonucleotides transfected cells FIG. 16B. The inset in FIG. 16B shows the AR expression as determined by Western blot analysis after HLEC transfections; C; control, L; treated with lipofectamine alone, S; treated with scrambled oligonucleotide and A, antisense oligonucleotide. Corresponding levels of the house-keeping enzyme protein glyceraldehydes-3-phosphate dehydrogenase (GAPDH), determined by Western analysis of the same gel are also shown in the inset.

FIG. 17A and FIG. 17B: Transient transfection of antisense AR prevents high glucose or TNF-α-induced apoptosis of HLEC. Quiescent HLEC were either incubated with lipofectamine or transfected with AR antisense or scrambled oligonucleotides as described in the text. After transfection, the cells were stimulated with 50 mM glucose or 2 nM TNF-α for an additional 24 h and the number of cells FIG. 17A and MTT reactivity FIG. 17B were measured. Bars represent mean±SE (n=4). **P<0.001 compared with the values obtained with cells transfected with the scrambled control oligonucleotide.

### DETAILED DESCRIPTION OF EMBODIMENTS

Abnormal vascular smooth muscle cell (VSMC) proliferation is a key feature of atherosclerosis and restenosis, however, the mechanisms regulating growth remain unclear. Various embodiments of the invention include compositions and methods for the inhibition of the aldehyde-metabolizing enzyme aldose reductase (AR), that for example, inhibits NF-κB activation during restenosis of balloon-injured rat carotid arteries as well as VSMC proliferation due to tumor necrosis factor (TNF-α) stimulation. Inhibition of VSMC growth by AR inhibitors was not accompanied by increase in cell death or apoptosis. Inhibition of AR led to a decrease in the activity of the transcription factor NF-κB in culture and in the neointima of rat carotid arteries after balloon injury. Inhibition of AR in VSMC also prevented the activation of NF-κB by fibroblast growth factor (bFGF), Angiotensin-II (Ang-II) and platelet-derived growth factor (PDGF-AB). The VSMC treated with AR inhibitors showed decreased nuclear translocation of NF-κB, and diminished phosphorylation and proteolytic degradation of IκB-α. Under identical conditions, treatment with AR inhibitors also prevented the activation of protein kinase C (PKC) by TNF-α, bFGF, Ang-II, and PDGF-AB but not phorbol esters, indicating that AR inhibitors prevent PKC stimulation or the availability of its activator, but not PKC itself. Treatment with antisense AR, which decreased the AR activity by >80%, attenuated PKC activation in TNF-α, bFGF, Ang-II, and PDGF-AB-stimulated VSMC and prevented TNF-α-induced proliferation. Collectively, these data suggest that inhibition of NF-κB may be a significant cause of the antimitogenic effects of AR inhibition and that this may be related to disruption of PKC-associated signaling in the AR-inhibited cells.

In additional embodiments of the invention compositions and methods are described for inhibition of AR. An increase in the flux of glucose through the polyol pathway has been suggested to be a significant source of tissue injury and dysfunction associated with long-term diabetes. The first and the rate-limiting step in the polyol pathway is catalyzed by aldose reductase (AR) that converts glucose to sorbitol. AR is a redox-sensitive protein, which is readily modified in vitro by oxidants including NO-donors and nitrosothiols. Therefore, we tested the hypothesis that NO may be a physiological regulator of AR and consequently the polyol pathway. We found that administration of the nitric oxide synthase (NOS) inhibitor—N^{G}-nitro-L-arginine methyl ester (L-NAME) increased sorbitol accumulation in the aorta of non-diabetic as well as diabetic rats, whereas treatment with L-arginine (a precursor of NO) or nitroglycerine patches prevented sorbitol accumulation. When incubated ex vivo with high glucose, sorbitol accumulation was increased by L-NAME and prevented by L-arginine in strips of aorta from rats or wild type, but not eNOS-deficient, mice. Also, exposure to NO-donors inhibited AR and prevented sorbitol accumulation in rat aortic vascular smooth muscle cells (VSMC) in culture. The NO-donors also increased the incorporation of radioactivity in the AR protein immunoprecipitated from VSMC in which the glutathione pool was labeled with [³⁵S]-cysteine. Based on these results, we conclude that NO regulates the vascular synthesis of polyols by S-thiolating AR. The observations suggest that increasing the synthesis or bioavailability of NO could prevent diabetic changes in polyol metabolism of glucose.

The inventors, therefore, examined the participation of AR in VSMC mitogenesis in response to TNF-α, which is the main mitogen driving neointima formation in vivo (Rectenwald et al., 2000; Niemann-Jonsson et al., 2001) and various growth factors.

I. AR and Diabetes Mellitus

Diabetes mellitus is characterized by abnormal glucose metabolism, which is usually associated with elevated levels of blood glucose (Ruderman et al., 1992; Wu, 1993; King et al., 1996). Although due to insulin deficiency or resistance, glucose utilization is diminished in tissues that require insulin for glucose uptake, tissues in which glucose transport is not regulated by insulin face severe and sustained hyperglycemia (Litherland et al., 2001; Czech and Corvera, 1999). Because glycolytic utilization is saturated, excessive glucose in these tissues is converted to sorbitol via NADPH-dependent reduction catalyzed by aldose reductase (AR). Under normal, euglycemic conditions, sorbitol synthesis represents a minor (>3%) fate of glucose in non-renal tissues, however, at levels encountered during diabetes 30 to 35% of the glucose could be converted to sorbitol. This increase in the polyol pathway has been linked to several pathological changes in insulin-insensitive tissues such as those in the blood vessels, peripheral nerves, renal medulla, blood cells and ocular lens. Although the mechanism by which the increase in the polyol pathway contributes to hyperglycemic injury are not well understood, it has been suggested that the osmotic and/or oxidative stress imposed by sorbitol accumulation or NADPH depletion may be significant biochemical changes contributing to the observed pathological changes (Burg, 1995; Hotta, 1997).

That a component of hyperglycemic injury is due to the increase in the polyol pathway activity is supported by extensive evidence showing that inhibition of AR prevents diabetic nephropathy, neuropathy, and cataract in rats (Jez et al., 1997; Kador et al., 1985). The contribution of AR to hyperglycemic injury is further supported by the observation that lens-specific overexpression of AR accelerates diabetic cataracts in mice (Lee et al., 1995). Nevertheless, the clinical utility of AR inhibitors in treating secondary diabetic complications remains unclear. Although, some of the variable clinical outcomes may be related to inappropriate dosing and hypersensitivity of selected individuals, the limited long-term efficacy of these drugs may be, in part, due to post-translational changes in AR which alter ligand binding and catalysis. The previous studies have shown that AR isolated from diabetic tissues displayed altered kinetic properties and was relatively insensitive to hydantoin inhibitors such as sorbinil as compared to the enzyme from normal tissues (Srivastava et al., 1985). Similar changes in kinetic and ligand-binding properties of AR were obtained upon in vitro thiol modification of the enzyme by hydrogen peroxide (H₂O₂) or NO, indicating that the intracellular activity of AR may be regulated by redox-sensitive reactions.

The high sensitivity of AR to oxidants such as H₂O₂ and NO is due to a reactive cysteine (Cys-298) present at the active site of the enzyme (Liu et al., 1993). The inventors have shown that Cys-298 is readily modified by NO-donors and that depending upon the conditions of the reaction and the nature of the NO-donor used, the enzyme is either S-thiolated or S-nitrosated (Chandra et al., 1997; Srivastava et al., 2001). On the basis of these observations The inventors hypothesized that NO regulates intracellular activity of AR and consequently the flux of glucose via the polyol pathway. To test this hypothesis, The inventors examined whether changes in NO synthesis or bioavailability affect AR activity or sorbitol synthesis in aorta from diabetic or non-diabetic animals. The results show that NO inactivates AR and inhibits sorbitol synthesis, and that this may relate to reversible S-thiolation of AR.

II. AR and Cardiovascular Disease

Cardiovascular complications are the major cause of morbidity and mortality in diabetes. Atherosclerosis is a multifactorial disease that results in endothelial dysfunction, abnormal proliferation of vascular smooth muscle cells and plaque formation Mitchell et al., 1998). These changes occlude blood flow and spontaneous plaque rupture leads to clinical symptoms of myocardial infarction and stroke. The process of atherosclerosis is accelerated by diabetes and the diabetic subjects have an increased risk of developing atherosclerotic disease (Kirpichnikov et al., 2001). Increased generation of reactive oxygen species (ROS) along with elevated levels of lipid peroxidation products such as α-β-unsaturated lipid aldehyde, 4-hydroxy-trans-nonenal (HNE) that accelerate vascular smooth muscle cell (VSMC) growth is considered to be one of the major factors underlying the increased incidence of atherosclerosis in diabetics (Yamanouchi et al., 2000; Cai et al., 2000). Previous studies suggest that the enzyme aldose reductase (AR), which catalyzes the reduction of glucose to sorbitol, represents a significant metabolic component in the development of secondary diabetic complications (Yabe-Nishimura, 1998). However, in addition to reducing glucose this enzyme also catalyzes the reduction of a broad range of aromatic and aliphatic aldehydes, particularly the atherogenic aldehydes that are generated during lipid peroxidation (Srivastava et al., 1999; Ramana et al., 2000; Srivastava et al., 2001). It was demonstrated that the active site of AR forms a glutathione-binding domain, which specifically recognizes and reduces glutathiolated aldehydes with high affinity (Ramana et al., 2000).

Aldose reductase constitutes the first and rate-limiting step of the polyol pathway and plays a central role in renal osmoregulation. The accelerated flux of sorbitol through the polyol pathway and enhanced oxidative stress is implicated in the pathogenesis of the secondary diabetic complications, such as cataractogenesis, retinopathy, neuropathy, nephropathy, and atherosclerosis (Yabe-Nishimura, 1998). It has been proposed that the increased flux of glucose via polyol pathway causes osmotic and oxidative stress, which, in turn, triggers a sequence of metabolic changes resulting in gross tissue dysfunction, altered intracellular signaling, and extensive cell death (Bucala, 1997). This view is supported by the observations that inhibition of AR prevents or delays several pleiotropic complications of diabetes such as cataractogenesis, retinopathy, neuropathy and nephropathy, and in transgenic mice, lens-specific overexpression of AR accelerates sugar cataract (Yabe-Nishimura, 1998; Lee et al., 1995). Nonetheless, the clinical utility of AR inhibitors remains uncertain. In several studies, inhibitors of AR do not interrupt or reverse progressive hyperglycemic injury. Moreover, unlike the cataractous lens, nerves or kidneys of diabetics do not accumulate high concentrations of sorbitol, yet they show functional improvement upon inhibition of AR.

The elevated ROS levels in hyperglycemia are known to trigger the inflammatory response in the tissues by upregulating several redox-sensitive kinases such as MAP kinase, protein kinase-C and also regulate transcription of several genes such as, TNF-α, IL-8 and AR by activating specific transcription factors (Koya et al. 1998; Rabinovitch, 1998). A major signaling pathway associated with the oxidative stress and inflammation is the activation of redox-sensitive nuclear factor-kappa binding protein (NF-κB). Modulation of NF-κB plays a central role in the mitogenic process initiated by ROS and related oxidants (Aggarwal, 2000).

III. Proteinaceous Compositions

Proteinaceous compositions are involved in screening, prognostic and treatment methods of the invention. The present embodiment of the invention contemplates inhibitors of aldose reductase, which is a proteinaceous composition, and the inhibitors are proteinaceous compositions in some embodiments of the invention. Furthermore, some of the screening methods can involve proteinaceous compositions such as TNFα, NK-κB, I-κB (proteins involved in screens that are not AR are referred herein as “screening proteins”). In this application, the amino acid sequence of an aldose reductase protein is involved. Furthermore, in some embodiments of the invention, proteinaceous compositions are used to identify candidate aldose reductase inhibitors. It is contemplated that any teaching with respect to one particular proteinaceous composition may apply generally to other proteinaceous compositions described herein.

As used herein, a “proteinaceous molecule,” “proteinaceous composition,” “proteinaceous compound,” “proteinaceous chain” or “proteinaceous material” generally refers, but is not limited to, a protein of greater than about 200 amino acids or the full length endogenous sequence translated from a gene; a polypeptide of greater than about 100 amino acids; and/or a peptide of from about 3 to about 100 amino acids. All the “proteinaceous” terms described above may be used interchangeably herein.

In certain embodiments of the invention, the proteinaceous composition may include such molecules that bear the size of at least one proteinaceous molecules that may comprise but is not limited to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 275, 300, 325, 350, 375, 383, 385 or greater amino molecule residues, and any range derivable therein. Such lengths are applicable to all polypeptides and peptides mentioned herein. It is contemplated that an aldose reductase inhibitor may specifically bind or recognize a particular region of AR, including 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 275, 300, 325, 350, 375, 383, 385 or greater contiguous amino acids of aldose reductase or any range of numbers of contiguous amino acids derivable therein. Aldose reductase may be from any organism, including mammals, such as a human, monkey, mouse, rat, hamster, cow, pig, rabbit, and may be from other cultured cells readily available. AR inhibitors may also affect polypeptides in pathways involving AR but found further upstream or downstream from AR in the pathway.

As used herein, an “amino molecule” refers to any amino acid, amino acid derivative or amino acid mimic as would be known to one of ordinary skill in the art. In certain embodiments, the residues of the proteinaceous molecule are sequential, without any non-amino molecule interrupting the sequence of amino molecule residues. In other embodiments, the sequence may comprise one or more non-amino molecule moieties. In particular embodiments, the sequence of residues of the proteinaceous molecule may be interrupted by one or more non-amino molecule moieties.

The term “functionally equivalent codon” is used herein to refer to codons that encode the same amino acid, such as the six codons for arginine and serine, and also refers to codons that encode biologically equivalent amino acids. Codon usage for various organisms and organelles can be found in codon usage databases, including, for example that made available by Nakamura (2002), which allows one of skill in the art to optimize codon usage for expression in various organisms using the disclosures herein. Thus, it is contemplated that codon usage may be optimized for other animals, as well as other organisms such as a prokaryote (e.g., an eubacteria, an archaea), an eukaryote (e.g., a protist, a plant, a fungi, an animal), a virus and the like, as well as organelles that contain nucleic acids, such as mitochondria, chloroplasts and the like, based on the preferred codon usage as would be known to those of ordinary skill in the art.

It will also be understood that amino acid sequences or nucleic acid sequences of AR, AR polypeptide inhibitors, or screening proteins may include additional residues, such as additional N- or C-terminal amino acids or 5′ or 3′ sequences, or various combinations thereof, and yet still be essentially as set forth in one of the sequences disclosed herein, so long as the sequence meets the criteria set forth above, including the maintenance of biological protein, polypeptide or peptide activity where expression of a proteinaceous composition is concerned. The addition of terminal sequences particularly applies to nucleic acid sequences that may, for example, include various non-coding sequences flanking either of the 5′ and/or 3′ portions of the coding region or may include various internal sequences, i.e., introns, which are known to occur within genes. In some embodiments, the C-terminal or N-terminal of the MIC polypeptide may also be glycosylated. It will be further understood that proteins of the invention may also be truncated or used as part of a chimeric protein, such as a fusion protein.

Proteinaceous compositions may be made by any technique known to those of skill in the art, including the expression of proteins, polypeptides or peptides through standard molecular biological techniques, the isolation of proteinaceous compounds from natural sources, or the chemical synthesis of proteinaceous materials. The nucleotide and protein, polypeptide and peptide sequences for various genes have been previously disclosed, and may be found at computerized databases known to those of ordinary skill in the art. For example, the Genbank and GenPept databases are available from the National Center for Biotechnology Information and are available online at the webpage for NCBI National Library of Medicine at the NIH (NCBI webpage, 2002). The coding regions for these known genes may be amplified and/or expressed using the techniques disclosed herein or as would be known to those of ordinary skill in the art. Alternatively, various commercial preparations of proteins, polypeptides and peptides are known to those of skill in the art.

In certain embodiments a proteinaceous compound may be purified. Generally, “purified” will refer to a specific or protein, polypeptide, or peptide composition that has been subjected to fractionation to remove various other proteins, polypeptides, or peptides, and which composition substantially retains its activity, as may be assessed, for example, by the protein assays, as would be known to one of ordinary skill in the art for the specific or desired protein, polypeptide or peptide. Polypeptides may also be “recombinant” meaning it was produced directly or indirectly (as from subsequent replication) from a nucleic acid that has been manipulated using recombinant DNA technology.

Recombinant vectors and isolated nucleic acid segments may variously include the coding regions themselves, coding regions bearing selected alterations or modifications in the basic coding region, and they may encode larger polypeptides or peptides that nevertheless include such coding regions or may encode biologically functional equivalent proteins, polypeptide or peptides that have variant amino acids sequences.

The nucleic acids of the present invention encompass biologically functional equivalent MIC proteins, polypeptides, or peptides, as well as MIC polypeptide binding agents, and detection agents. Such sequences may arise as a consequence of codon redundancy or functional equivalency that are known to occur naturally within nucleic acid sequences or the proteins, polypeptides or peptides thus encoded. Alternatively, functionally equivalent proteins, polypeptides or peptides may be created via the application of recombinant DNA technology, in which changes in the protein, polypeptide or peptide structure may be engineered, based on considerations of the properties of the amino acids being exchanged. Recombinant changes may be introduced, for example, through the application of site-directed mutagenesis techniques as discussed herein below, e.g., to introduce improvements or alterations to the antigenicity of the protein, polypeptide or peptide, or to test mutants in order to examine MIC protein, polypeptide, or peptide activity at the molecular level.

Another embodiment for the preparation of polypeptides according to the invention is the use of peptide mimetics. Peptide mimetics may be screened as a candidate substance. Mimetics are peptide-containing compounds, that mimic elements of protein secondary structure. The underlying rationale behind the use of peptide mimetics is that the peptide backbone of proteins exists chiefly to orient amino acid side chains in such a way as to facilitate molecular interactions, such as those of antibody and antigen. A peptide mimetic is expected to permit molecular interactions similar to the natural molecule. These principles may be used, in conjunction with the principles outlined above, to engineer second generation molecules having many of the natural properties of AR inhibitors, but with altered and even improved characteristics.

Sequence variants of the polypeptide, as mentioned above, can be prepared. These may, for instance, be minor sequence variants of the polypeptide that arise due to natural variation within the population or they may be homologues found in other species. They also may be sequences that do not occur naturally but that are sufficiently similar that they function similarly and/or elicit an immune response that cross-reacts with natural forms of the polypeptide. Sequence variants can be prepared by standard methods of site-directed mutagenesis such as those described below in the following section.

Amino acid sequence variants of the polypeptide can be substitutional, insertional or deletion variants. Deletion variants lack one or more residues of the native protein which are not essential for function or immunogenic activity, and are exemplified by the variants lacking a transmembrane sequence described above. Another common type of deletion variant is one lacking secretory signal sequences or signal sequences directing a protein to bind to a particular part of a cell.

Substitutional variants typically contain the exchange of one amino acid for another at one or more sites within the protein, and may be designed to modulate one or more properties of the polypeptide such as stability against proteolytic cleavage. Substitutions preferably are conservative, that is, one amino acid is replaced with one of similar shape and charge. Conservative substitutions are well known in the art and include, for example, the changes of: alanine to serine; arginine to lysine; asparagine to glutamine or histidine; aspartate to glutamate; cysteine to serine; glutamine to asparagine; glutamate to aspartate; glycine to proline; histidine to asparagine or glutamine; isoleucine to leucine or valine; leucine to valine or isoleucine; lysine to arginine; methionine to leucine or isoleucine; phenylalanine to tyrosine, leucine or methionine; serine to threonine; threonine to serine; tryptophan to tyrosine; tyrosine to tryptophan or phenylalanine; and valine to isoleucine or leucine.

Insertional variants include fusion proteins such as those used to allow rapid purification of the polypeptide and also can include hybrid proteins containing sequences from other proteins and polypeptides which are homologues of the polypeptide. For example, an insertional variant could include portions of the amino acid sequence of the polypeptide from one species, together with portions of the homologous polypeptide from another species. Other insertional variants can include those in which additional amino acids are introduced within the coding sequence of the polypeptide. These typically are smaller insertions than the fusion proteins described above and are introduced, for example, into a protease cleavage site.

Modification and changes may be made in the structure of a gene and still obtain a functional molecule that encodes a protein or polypeptide with desirable characteristics. The following is a discussion based upon changing the amino acids of a protein to create an equivalent, or even an improved, second-generation molecule.

Site-specific mutagenesis is a technique useful in the preparation of individual peptides, or biologically functional equivalent proteins or peptides, through specific mutagenesis of the underlying DNA. The technique further provides a ready ability to prepare and test sequence variants, incorporating one or more of the foregoing considerations, by introducing one or more nucleotide sequence changes into the DNA. Site-specific mutagenesis allows the production of mutants through the use of specific oligonucleotide sequences which encode the DNA sequence of the desired mutation, as well as a sufficient number of adjacent nucleotides, to provide a primer sequence of sufficient size and sequence complexity to form a stable duplex on both sides of the deletion junction being traversed. Typically, a primer of about 17 to 25 nucleotides in length is preferred, with about 5 to 10 nucleotides on both sides of the junction of the sequence being altered.

Within certain embodiments expression vectors are employed to express various genes to produce large amounts of AR polypeptide product, AR inhibitors, screening proteins, or any other proteinaceous composition for use with the invention, which can then be purified. Expression requires that appropriate signals be provided in the vectors, and which include various regulatory elements, such as enhancers/promoters from both viral and mammalian sources that drive expression of the genes of interest in host cells. Elements designed to optimize messenger RNA stability and translatability in host cells also are required. The conditions for the use of a number of dominant drug selection markers for establishing permanent, stable cell clones expressing the proteinaceous products are also required, as is an element that links expression of the drug selection markers to expression of the polypeptide.

In certain embodiments of the invention, it will be desirable to produce a functional AR polypeptide, AR polypeptide inhibitors, screening proteins, or variants thereof. Protein purification techniques are well known to those of skill in the art. These techniques tend to involve the fractionation of the cellular milieu to separate AR or related polypeptides from other components of the mixture. Having separated AR and related polypeptides from the other plasma components, the AR or related polypeptide sample may be purified using chromatographic and electrophoretic techniques to achieve complete purification. Analytical methods particularly suited to the preparation of a pure peptide are ion-exchange chromatography, exclusion chromatography; polyacrylamide gel electrophoresis; isoelectric focusing. A particularly efficient method of purifying peptides is fast protein liquid chromatography or even HPLC.

Certain aspects of the present invention concern the purification, and in particular embodiments, the substantial purification, of an encoded protein or peptide. The term “purified protein or peptide” as used herein, is intended to refer to a composition, isolatable from other components, wherein the protein or peptide is purified to any degree relative to its naturally-obtainable state, i.e., in this case, relative to its purity within a VEC or VSMC. A purified protein or peptide therefore also refers to a protein or peptide, free from the environment in which it may naturally occur. It is contemplated that purification of human AR can be achieved using the protocol of Chandra et al., 1997, which is specifically incorporated by reference.

Generally, “purified” will refer to a protein or peptide composition that has been subjected to fractionation to remove various other components, and which composition substantially retains its expressed biological activity. Where the term “substantially purified” is used, this designation will refer to a composition in which the protein or peptide forms the major component of the composition, such as constituting about 50% or more of the proteins in the composition.

There is no general requirement that the protein or peptide always be provided in their most purified state. Indeed, it is contemplated that less substantially purified products will have utility in certain embodiments. Partial purification may be accomplished by using fewer purification steps in combination, or by utilizing different forms of the same general purification scheme. For example, it is appreciated that a cation-exchange column chromatography performed utilizing an HPLC apparatus will generally result in a greater-fold purification than the same technique utilizing a low pressure chromatography system. Methods exhibiting a lower degree of relative purification may have advantages in total recovery of protein product, or in maintaining the activity of an expressed protein.

The present invention also describes the synthesis of peptides that can directly or indirectly inhibit AR. Because of their relatively small size, the peptides of the invention can also be synthesized in solution or on a solid support in accordance with conventional techniques. Various automatic synthesizers are commercially available and can be used in accordance with known protocols. See, for example, Stewart and Young, (1984); Tam et al., (1983); Merrifield, (1986); and Barany and Merrifield (1979). Short peptide sequences, or libraries of overlapping peptides, usually from about 6 up to about 35 to 50 amino acids, which correspond to the selected regions described herein, can be readily synthesized and then screened in screening assays designed to identify reactive peptides. Alternatively, recombinant DNA technology may be employed wherein a nucleotide sequence which encodes a peptide of the invention is inserted into an expression vector, transformed or transfected into an appropriate host cell and cultivated under conditions suitable for expression.

In some embodiments of the present invention, the use of binding agents that are immunoreactive with AR or a screening protein, or any portion thereof is contemplated. Any of the discussion regarding proteinaceous compositions may be applied to antibodies as well.

Binding agents include polyclonal or monoclonal antibodies and fragments thereof. In a preferred embodiment, an antibody is a monoclonal antibody. The following monoclonal antibodies of the present invention were prepared against MICA (2C10 and 3H5) and against MICA and MICB (6D4 and 6G6), Such antibodies may form part of an immunodetection kit as described herein below.

Means for preparing and characterizing antibodies are well known in the art (See, e.g., Harlow and Lane, 1988).

In the present invention, it is further contemplated that the antibody may be linked to a second antibody which may bind to a different epitope than the first antibody.

IV. Nucleic Acids

Proteins used in the context of the invention may be expressed from a cDNA. The engineering of DNA segment(s) for expression in a prokaryotic or eukaryotic system may be performed by techniques generally known to those of skill in recombinant expression. It is believed that virtually any expression system may be employed in the expression of the claimed nucleic acid sequences.

The term “nucleic acid” is well known in the art. A “nucleic acid” as used herein will generally refer to a molecule (i.e., a strand) of DNA, RNA or a derivative or analog thereof, comprising a nucleobase. A nucleobase includes, for example, a naturally occurring purine or pyrimidine base found in DNA (e.g., an adenine “A,” a guanine “G,” a thymine “T” or a cytosine “C”) or RNA (e.g., an A, a G, an uracil “U” or a C). The term “nucleic acid” encompass the terms “oligonucleotide” and “polynucleotide,” each as a subgenus of the term “nucleic acid.” The term “oligonucleotide” refers to a molecule of between about 3 and about 100 nucleobases in length. The term “polynucleotide” refers to at least one molecule of greater than about 100 nucleobases in length.

These definitions generally refer to a single-stranded molecule, but in specific embodiments will also encompass an additional strand that is partially, substantially or fully complementary to the single-stranded molecule. Thus, a nucleic acid may encompass a double-stranded molecule or a triple-stranded molecule that comprises one or more complementary strand(s) or “complement(s)” of a particular sequence comprising a molecule. As used herein, a single stranded nucleic acid may be denoted by the prefix “ss,” a double stranded nucleic acid by the prefix “ds,” and a triple stranded nucleic acid by the prefix “ts.”

In one embodiment, the nucleic acid sequences complementary to at least a portion of the nucleic acid encoding AR will find utility as AR inhibitors. Hybridization is particularly useful in the detection of cDNA clones derived from sources where an extremely low amount of mRNA sequences relating to the polypeptide of interest are present. In other words, by using stringent hybridization conditions directed to avoid non-specific binding, it is possible, for example, to allow the autoradiographic visualization of a specific cDNA done by the hybridization of the target DNA to that single probe in the mixture which is its complete complement (Wallace et al., 1981). The use of a probe or primer of between 13 and 100 nucleotides, preferably between 17 and 100 nucleotides in length, or in some aspects of the invention up to 1-2 kilobases or more in length, allows the formation of a duplex molecule that is both stable and selective. These nucleic acids may be used, for example, in diagnostic evaluation of tissue samples or employed to clone full length cDNAs or genomic clones corresponding thereto. In certain embodiments, these probes consist of oligonucleotide fragments. Such fragments should be of sufficient length to provide specific hybridization to a RNA or DNA tissue sample. The sequences typically will be 10-20 nucleotides, but may be longer. Longer sequences, e.g., 40, 50, 100, 500 and even up to full length, are preferred for certain embodiments.

DNA segments encoding a specific gene may be introduced into recombinant host cells and employed for expressing a specific structural or regulatory protein. Alternatively, through the application of genetic engineering techniques, subportions or derivatives of selected genes may be employed. Upstream regions containing regulatory regions such as promoter regions may be isolated and subsequently employed for expression of the selected gene.

A non-limiting example of an enzymatically produced nucleic acid include one produced by enzymes in amplification reactions such as PCR™ (see for example, U.S. Pat. Nos. 4,683,202 and 4,682,195), or the synthesis of an oligonucleotide described in U.S. Pat. No. 5,645,897. A non-limiting example of a biologically produced nucleic acid includes a recombinant nucleic acid produced (i.e., replicated) in a living cell, such as a recombinant DNA vector replicated in bacteria (see for example, Sambrook et al. 1989). A nucleic acid may be purified on polyacrylamide gels, cesium chloride centrifugation gradients, or by any other means known to one of ordinary skill in the art (see for example, Sambrook et al., 1989).

To express a recombinant encoded protein or peptide, whether mutant or wild-type, in accordance with the present invention one would prepare an expression vector that comprises an AR-encoding nucleic acids, or a nucleic acid that encodes an AR inhibitor or a screening protein, under the control of, or operatively linked to, one or more promoters. To bring a coding sequence “under the control of” a promoter, one positions the 5′ end of the transcription initiation site of the transcriptional reading frame generally between about 1 and about 50 nucleotides “downstream” (i.e., 3′) of the chosen promoter. The “upstream” promoter stimulates transcription of the DNA and promotes expression of the encoded recombinant protein. This is the meaning of “recombinant expression” in this context.

In order to mediate the effect transgene expression in a cell, it will be necessary to transfer the therapeutic expression constructs of the present invention into a cell. Such transfer may employ viral or non-viral methods of gene transfer. This section provides a discussion of methods and compositions of gene transfer.

Viral vectors that may be used include, but are not limited to, adenovirus, adeno-associated virus, retrovirus, herpesvirus, papilloma virus, vaccinia virus, or hepatitis virus.

DNA constructs of the present invention are generally delivered to a cell, in certain situations, the nucleic acid to be transferred is non-infectious, and can be transferred using non-viral methods. Several non-viral methods for the transfer of expression constructs into cultured mammalian cells are contemplated by the present invention. These include calcium phosphate precipitation (Graham and Van Der Eb, 1973; Chen and Okayama, 1987; Rippe et al., 1990) DEAE-dextran (Gopal, 1985), electroporation (Tur-Kaspa et al., 1986; Potter et al., 1984), direct microinjection (Harland and Weintraub, 1985), DNA-loaded liposomes (Nicolau and Sene, 1982; Fraley et al., 1979), cell sonication (Fechheimer et al., 1987), gene bombardment using high velocity microprojectiles (Yang et al., 1990), and receptor-mediated transfection (Wu and Wu, 1987; Wu and Wu, 1988).

Antisense methodology takes advantage of the fact that nucleic acids tend to pair with “complementary” sequences. By complementary, it is meant that polynucleotides are those which are capable of base-pairing according to the standard Watson-Crick complementarity rules. That is, the larger purines will base pair with the smaller pyrimidines to form combinations of guanine paired with cytosine (G:C) and adenine paired with either thymine (A:T) in the case of DNA, or adenine paired with uracil (A:U) in the case of RNA. Inclusion of less common bases such as inosine, 5-methylcytosine, 6-methyladenine, hypoxanthine and others in hybridizing sequences does not interfere with pairing.

Targeting double-stranded (ds) DNA with polynucleotides leads to triple-helix formation; targeting RNA will lead to double-helix formation. Antisense polynucleotides, when introduced into a target cell, specifically bind to their target polynucleotide and interfere with transcription, RNA processing, transport, translation and/or stability. Antisense RNA constructs, or DNA encoding such antisense RNAs, may be employed to inhibit gene transcription or translation or both within a host cell, either in vitro or in vivo, such as within a host animal, including a human subject.

Antisense constructs may be designed to bind to the promoter and other control regions, exons, introns or even exon-intron boundaries of a gene. It is contemplated that the most effective antisense constructs may include regions complementary to intron/exon splice junctions. Thus, antisense constructs with complementarity to regions within 50-200 bases of an intron-exon splice junction may be used. It has been observed that some exon sequences can be included in the construct without seriously affecting the target selectivity thereof. The amount of exonic material included will vary depending on the particular exon and intron sequences used. One can readily test whether too much exon DNA is included simply by testing the constructs in vitro to determine whether normal cellular function is affected or whether the expression of related genes having complementary sequences is affected.

As stated above, “complementary” or “antisense” means polynucleotide sequences that are substantially complementary over their entire length and have very few base mismatches. For example, sequences of fifteen bases in length may be termed complementary when they have complementary nucleotides at thirteen or fourteen positions. Naturally, sequences which are completely complementary will be sequences which are entirely complementary throughout their entire length and have no base mismatches. Other sequences with lower degrees of homology also are contemplated. For example, an antisense construct which has limited regions of high homology, but also contains a non-homologous region (e.g., ribozyme) could be designed. These molecules, though having less than 50% homology, would bind to target sequences under appropriate conditions.

It may be advantageous to combine portions of genomic DNA with cDNA or synthetic sequences to generate specific constructs. For example, where an intron is desired in the ultimate construct, a genomic clone will need to be used. The cDNA or a synthesized polynucleotide may provide more convenient restriction sites for the remaining portion of the construct and, therefore, would be used for the rest of the sequence.

The use of AR-specific ribozymes is claimed in the present application. The following information is provided in order to compliment the earlier section and to assist those of skill in the art in this endeavor.

Ribozymes are RNA-protein complexes that cleave nucleic acids in a site-specific fashion. Ribozymes have specific catalytic domains that possess endonuclease activity (Kim and Cech, 1987; Gerlack et al., 1987; Forster and Symons, 1987). For example, a large number of ribozymes accelerate phosphoester transfer reactions with a high degree of specificity, often cleaving only one of several phosphoesters in an oligonucleotide substrate (Cech et al., 1981; Michel and Westhof, 1990; Reinhold-Hurek and Shub, 1992). This specificity has been attributed to the requirement that the substrate bind via specific base-pairing interactions to the internal guide sequence (“IGS”) of the ribozyme prior to chemical reaction.

Ribozyme catalysis has primarily been observed as part of sequence specific cleavage/ligation reactions involving nucleic acids (Joyce, 1989; Cech et al., 1981). For example, U.S. Pat. No. 5,354,855 reports that certain ribozymes can act as endonucleases with a sequence specificity greater than that of known ribonucleases and approaching that of the DNA restriction enzymes. Thus, sequence-specific ribozyme-mediated inhibition of gene expression may be particularly suited to therapeutic applications (Scanlon et al., 1991; Sarver et al., 1990; Sioud et al., 1992). Recently, it was reported that ribozymes elicited genetic changes in some cell lines to which they were applied; the altered genes included the oncogenes H-ras, c-fos and genes of HIV. Most of this work involved the modification of a target mRNA, based on a specific mutant codon that is cleaved by a specific ribozyme. In light of the information included herein and the knowledge of one of ordinary skill in the art, the preparation and use of additional ribozymes that are specifically targeted to a given gene will now be straightforward.

Several different ribozyme motifs have been described with RNA cleavage activity (reviewed in Symons, 1992). Examples that would be expected to function equivalently for the down regulation of AR include sequences from the Group I self splicing introns including tobacco ringspot virus (Prody et al., 1986), avocado sunblotch viroid (Palukaitis et al., 1979 and Symons, 1981), and Lucerne transient streak virus (Forster and Symons, 1987). Sequences from these and related viruses are referred to as hammerhead ribozymes based on a predicted folded secondary structure.

Other suitable ribozymes include sequences from RNase P with RNA cleavage activity (Yuan et al., 1992, Yuan and Altman, 1994), hairpin ribozyme structures (Berzal-Herranz et al., 1992; Chowrira et al., 1993) and hepatitis δ virus based ribozymes (Perrotta and Been, 1992). The general design and optimization of ribozyme directed RNA cleavage activity has been discussed in detail (Haseloff and Gerlach, 1988, Symons, 1992, Chowrira, et al., 1994, and Thompson, et al., 1995).

The other variable on ribozyme design is the selection of a cleavage site on a given target RNA. Ribozymes are targeted to a given sequence by virtue of annealing to a site by complimentary base pair interactions. Two stretches of homology are required for this targeting. These stretches of homologous sequences flank the catalytic ribozyme structure defined above. Each stretch of homologous sequence can vary in length from 7 to 15 nucleotides. The only requirement for defining the homologous sequences is that, on the target RNA, they are separated by a specific sequence which is the cleavage site. For hammerhead ribozymes, the cleavage site is a dinucleotide sequence on the target RNA, uracil (U) followed by either an adenine, cytosine or uracil (A, C or U; Perriman, et al., 1992; Thompson, et al., 1995). The frequency of this dinucleotide occurring in any given RNA is statistically 3 out of 16. Therefore, for a given target messenger RNA of 1000 bases, 187 dinucleotide cleavage sites are statistically possible.

Designing and testing ribozymes for efficient cleavage of a target RNA is a process well known to those skilled in the art. Examples of scientific methods for designing and testing ribozymes are described by Chowrira et al., (1994) and Lieber and Strauss (1995), each incorporated by reference. The identification of operative and preferred sequences for use in AR-targeted ribozymes is simply a matter of preparing and testing a given sequence, and is a routinely practiced “screening” method known to those of skill in the art.

An RNA molecule capable of mediating RNA interference in a cell is referred to as “siRNA.” Elbashir et al. (2001) discovered a clever method to bypass the anti viral response and induce gene specific silencing in mammalian cells. Several 21-nucleotide dsRNAs with 2 nucleotide 3′ overhangs were transfected into mammalian cells without inducing the antiviral response. The small dsRNA molecules (also referred to as “siRNA”) were capable of inducing the specific suppression of target genes.

In the context of the present invention, siRNA directed against AR, NF-κB, and TNF-α are specifically contemplated. The siRNA can target a particular sequence because of a region of complementarity between the siRNA and the RNA transcript encoding the polypeptide whose expression will be decreased, inhibited, or eliminated.

An siRNA may be a double-stranded compound comprising two separate, but complementary strands of RNA or it may be a single RNA strand that has a region that self-hybridizes such that there is a double-stranded intramolecular region of 7 basepairs or longer (see Sui et al., 2002 and Brummelkamp et al., 2002 in which a single strand with a hairpin loop is used as a dsRNA for RNAi). In some cases, a double-stranded RNA molecule may be processed in the cell into different and separate siRNA molecules.

In some embodiments, the strand or strands of dsRNA are 100 bases (or basepairs) or less, in which case they may also be referred to as “siRNA.” In specific embodiments the strand or strands of the dsRNA are less than 70 bases in length. With respect to those embodiments, the dsRNA strand or strands may be from 5-70, 10-65, 20-60, 30-55, 40-50 bases or basepairs in length. A dsRNA that has a complementarity region equal to or less than 30 basepairs (such as a single stranded hairpin RNA in which the stem or complementary portion is less than or equal to 30 basepairs) or one in which the strands are 30 bases or fewer in length is specifically contemplated, as such molecules evade a mammalian's cell antiviral response. Thus, a hairpin dsRNA (one strand) may be 70 or fewer bases in length with a complementary region of 30 basepairs or fewer.

Methods of using siRNA to achieve gene silencing are discussed in WO 03/012052, which is specifically incorporated by reference herein. Designing and testing siRNA for efficient inhibition of expression of a target polypeptide is a process well known to those skilled in the art. Their use has become well known to those of skill in the art. The techniques described in U.S. Patent Publication No. 20030059944 and 20030105051 are incorporated herein by reference. Furthermore, a number of kits are commercially available for generating siRNA molecules to a particular target, which in this case includes AR, NF-κB, and TNF-α. Kits such as Silencer™ Express, Silencer™ siRNA Cocktail, Silencer™ siRNA Construction, MEGAScript® RNAi are readily available from Ambion, Inc.

Other candidate AR inhibitors include aptamers and aptazymes, which are synthetic nucleic acid ligands. The methods of the present invention may involve nucleic acids that modulate AR, NF-κB, and TNF-α. Thus, in certain embodiments, a nucleic acid, may comprise or encode an aptamer. An “aptamer” as used herein refers to a nucleic acid that binds a target molecule through interactions or conformations other than those of nucleic acid annealing/hybridization described herein. Methods for making and modifying aptamers, and assaying the binding of an aptamer to a target molecule may be assayed or screened for by any mechanism known to those of skill in the art (see for example, U.S. Pat. Nos. 5,840,867, 5,792,613, 5,780,610, 5,756,291 and 5,582,981, Burgstaller et al., 2002, which are incorporated herein by reference.

Another therapeutic embodiment of the present invention contemplates the use of single-chain antibodies to block the activity of AR, NF-κB, or TNF-α in cells. Single-chain antibodies can be synthesized by a cell, targeted to particular cellular compartments, and used to interfere in a highly specific manner with cell growth and metabolism (Richardson and Marasco, 1995).

Methods for the production of single-chain antibodies are well known to those of skill in the art. The skilled artisan is referred to U.S. Pat. No. 5,359,046, (incorporated herein by reference) for such methods. A single-chain antibody is created by fusing together the variable domains of the heavy and light chains using a short peptide linker, thereby reconstituting an antigen binding site on a single molecule.

V. Methods of Screening

The present invention also contemplates screening of compounds for activity in inhibiting AR. These assays may make use of a variety of different formats and may depend on the kind of “activity” for which the screen is being conducted. Contemplated functional “read-outs” include binding to a compound such as AR, NF-κB, or TNFα, inhibition of any or these protein's binding to a substrate, ligand, receptor or other binding partner by a compound, phosphatase activity, anti-phosphatase activity, post-translational modification of these proteins, inhibition or stimulation of apoptosis, cell signalling, transcriptional activation, DNA binding, or cytokine induction. Assays may be performed in vitro or in vivo, or both.

Determining the effectiveness of a compound in vivo may involve a variety of different criteria. Such criteria include, but are not limited to, survival, reduction of symptoms, and improvement in prognosis.

The goal of rational drug design is to produce structural analogs of biologically active polypeptides or compounds with which they interact (agonists, antagonists, inhibitors, binding partners, etc.). By creating such analogs, it is possible to fashion drugs which are more active or stable than the natural molecules, which have different susceptibility to alteration or which may affect the function of various other molecules.

One may design drugs that act as stimulators, inhibitors, agonists, antagonists of AR. By virtue of the availability of cloned AR sequences, sufficient amounts of AR can be produced to perform crystallographic studies. In addition, knowledge of the polypeptide sequences permits computer employed predictions of structure-function relationships.

VI. Pharmaceutical Compositions and Routes of Administration

Pharmaceutical compositions of the present invention may comprise an effective amount of one or more AR inhibitors, including NO inducers, (and/or an additional agents) dissolved or dispersed in a pharmaceutically acceptable carrier to a subject. The phrases “pharmaceutical or pharmacologically acceptable” refers to molecular entities and compositions that do not produce an adverse, allergic or other untoward reaction when administered to an animal, such as, for example, a human, as appropriate. The preparation of a pharmaceutical composition that contains at least one AR inhibitor or additional active ingredient will be known to those of skill in the art in light of the present disclosure, and as exemplified by Remington's Pharmaceutical Sciences, 18th Ed. Mack Printing Company, 1990, incorporated herein by reference. Moreover, for animal (e.g., human) administration, it will be understood that preparations should meet sterility, pyrogenicity, general safety and purity standards as required by FDA Office of Biological Standards.

As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18th Ed. Mack Printing Company, 1990, pp. 1289-1329, incorporated herein by reference). Except insofar as any conventional carrier is incompatible with the active ingredient, its use in the therapeutic or pharmaceutical compositions is contemplated.

A pharmaceutical composition of the present invention may comprise different types of carriers depending on whether it is to be administered in solid, liquid or aerosol form, and whether it needs to be sterile for such routes of administration as injection. A pharmaceutical composition of the present invention can be administered intravenously, intradermally, intraarterially, intraperitoneally, intraarticularly, intrapleurally, intranasally, topically, intramuscularly, intraperitoneally, subcutaneously, subconjunctival, intravesicularlly, mucosally, intrapericardially, intraumbilically, orally, topically, locally, inhalation (e.g., aerosol inhalation), injection, infusion, continuous infusion, via a catheter, via a lavage, in lipid compositions (e.g., liposomes), or by other method or any combination of the forgoing as would be known to one of ordinary skill in the art (see, for example, Remington's Pharmaceutical Sciences, 18th Ed. Mack Printing Company, 1990, incorporated herein by reference).

The actual dosage amount of a composition of the present invention administered to a subject can be determined by physical and physiological factors such as body weight, severity of condition, the type of disease being treated, previous or concurrent therapeutic interventions, idiopathy of the patient and on the route of administration. The number of doses and the period of time over which the dose may be given may vary. The practitioner responsible for administration will, in any event, determine the concentration of active ingredient(s) in a composition and appropriate dose(s), as well as the length of time for administration for the individual subject.

In certain embodiments, pharmaceutical compositions may comprise, for example, at least about 0.1% of an active compound. In other embodiments, the an active compound may comprise between about 2% to about 75% of the weight of the unit, or between about 25% to about 60%, for example, and any range derivable therein. In other non-limiting examples, a dose may also comprise from about 1 microgram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 mg/kg/body weight or more per administration, and any range derivable therein. In non-limiting examples of a derivable range from the numbers listed herein, a range of about 5 mg/kg/body weight to about 100 mg/kg/body weight, about 5 microgram/kg/body weight to about 500 milligram/kg/body weight, etc., can be administered, based on the numbers described above.

In any case, the composition may comprise various antioxidants to retard oxidation of one or more component. Additionally, the prevention of the action of microorganisms can be brought about by preservatives such as various antibacterial and antifungal agents, including but not limited to parabens (e.g., methylparabens, propylparabens), chlorobutanol, phenol, sorbic acid, thimerosal or combinations thereof.

An AR inhibitor(s) of the present invention may be formulated into a composition in a free base, neutral or salt form. Pharmaceutically acceptable salts, include the acid addition salts, e.g., those formed with the free amino groups of a proteinaceous composition, or which are formed with inorganic-acids such as for example, hydrochloric or phosphoric acids, or such organic acids as acetic, oxalic, tartaric or mandelic acid. Salts formed with the free carboxyl groups can also be derived from inorganic bases such as for example, sodium, potassium, ammonium, calcium or ferric hydroxides; or such organic bases as isopropylamine, trimethylamine, histidine or procaine.

In embodiments where the composition is in a liquid form, a carrier can be a solvent or dispersion medium comprising but not limited to, water, ethanol, polyol (e.g., glycerol, propylene glycol, liquid polyethylene glycol, etc), lipids (e.g., triglycerides, vegetable oils, liposomes) and combinations thereof. The proper fluidity can be maintained, for example, by the use of a coating, such as lecithin; by the maintenance of the required particle size by dispersion in carriers such as, for example liquid polyol or lipids; by the use of surfactants such as, for example hydroxypropylcellulose; or combinations thereof such methods. In many cases, it will be preferable to include isotonic agents, such as, for example, sugars, sodium chloride or combinations thereof.

In certain aspects of the invention, the AR inhibitors are prepared for administration by such routes as oral ingestion. In these embodiments, the solid composition may comprise, for example, solutions, suspensions, emulsions, tablets, pills, capsules (e.g., hard or soft shelled gelatin capsules), sustained release formulations, buccal compositions, troches, elixirs, suspensions, syrups, wafers, or combinations thereof. Oral compositions may be incorporated directly with the food of the diet. Preferred carriers for oral administration comprise inert diluents, assimilable edible carriers or combinations thereof. In other aspects of the invention, the oral composition may be prepared as a syrup or elixir. A syrup or elixir, and may comprise, for example, at least one active agent, a sweetening agent, a preservative, a flavoring agent, a dye, a preservative, or combinations thereof.

In certain preferred embodiments an oral composition may comprise one or more binders, excipients, disintegration agents, lubricants, flavoring agents, and combinations thereof. In certain embodiments, a composition may comprise one or more of the following: a binder, such as, for example, gum tragacanth, acacia, cornstarch, gelatin or combinations thereof, an excipient, such as, for example, dicalcium phosphate, mannitol, lactose, starch, magnesium stearate, sodium saccharine, cellulose, magnesium carbonate or combinations thereof; a disintegrating agent, such as, for example, corn starch, potato starch, alginic acid or combinations thereof; a lubricant, such as, for example, magnesium stearate; a sweetening agent, such as, for example, sucrose, lactose, saccharin or combinations thereof; a flavoring agent, such as, for example peppermint, oil of wintergreen, cherry flavoring, orange flavoring, etc.; or combinations thereof the foregoing. When the dosage unit form is a capsule, it may contain, in addition to materials of the above type, carriers such as a liquid carrier. Various other materials may be present as coatings or to otherwise modify the physical form of the dosage unit. For instance, tablets, pills, or capsules may be coated with shellac, sugar or both.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and/or the other ingredients. In the case of sterile powders for the preparation of sterile injectable solutions, suspensions or emulsion, the preferred methods of preparation are vacuum-drying or freeze-drying techniques which yield a powder of the active ingredient plus any additional desired ingredient from a previously sterile-filtered liquid medium thereof. The liquid medium should be suitably buffered if necessary and the liquid diluent first rendered isotonic prior to injection with sufficient saline or glucose. The preparation of highly concentrated compositions for direct injection is also contemplated, where the use of DMSO as solvent is envisioned to result in extremely rapid penetration, delivering high concentrations of the active agents to a small area.

The composition must be stable under the conditions of manufacture and storage, and preserved against the contaminating action of microorganisms, such as bacteria and fungi. It will be appreciated that endotoxin contamination should be kept minimally at a safe level, for example, less that 0.5 ng/mg protein.

In particular embodiments, prolonged absorption of an injectable composition can be brought about by the use in the compositions of agents delaying absorption, such as, for example, aluminum monostearate, gelatin or combinations thereof.

In order to increase the effectiveness of treatments with the compositions of the present invention, such as an AR inhibitor, it may be desirable to combine it with other therapeutic agents. This process may involve contacting the cell(s) with an AR inhibitor and a therapeutic agent at the same time or within a period of time wherein separate administration of the modulator and an agent to a cell, tissue or organism produces a desired therapeutic benefit. The terms “contacted” and “exposed,” when applied to a cell, tissue or organism, are used herein to describe the process by which a AR inhibitor and/or therapeutic agent are delivered to a target cell, tissue or organism or are placed in direct juxtaposition with the target cell, tissue or organism. The cell, tissue or organism may be contacted (e.g., by administration) with a single composition or pharmacological formulation that includes both a AR inhibitor and one or more agents, or by contacting the cell with two or more distinct compositions or formulations, wherein one composition includes an AR inhibitor and the other includes one or more agents.

The AR inhibitor may precede, be concurrent with and/or follow the other agent(s) by intervals ranging from minutes to weeks. In embodiments where the AR inhibitor and other agent(s) are applied separately to a cell, tissue or organism, one would generally ensure that a significant period of time did not expire between the time of each delivery, such that the inhibitor and agent(s) would still be able to exert an advantageously combined effect on the cell, tissue or organism. For example, in such instances, it is contemplated that one may contact the cell, tissue or organism with two, three, four or more modalities substantially simultaneously (i.e., within less than about a minute) as the modulator. In other aspects, one or more agents may be administered within of from substantially simultaneously, about 1 minute, about 5 minutes, about 10 minutes, about 20 minutes about 30 minutes, about 45 minutes, about 60 minutes, about 2 hours, or more hours, or about 1 day or more days, or about 4 weeks or more weeks, or about 3 months or more months, or about one or more years, and any range derivable therein, prior to and/or after administering the AR inhibitor.

Various combinations of a AR inhibitor(s) and a second therapeutic may be employed in the present invention, where a AR inhibitor is “A” and the secondary agent, such as a diabetic treatment, is “B”:

A/B/A B/A/B B/B/A A/A/B A/B/B B/A/A B/B/B/A B/B/A/B A/A/B/B A/B/A/B A/B/B/A B/B/A/A B/A/B/A B/A/A/B A/A/A/B B/A/A/A A/B/A/A A/A/B/A A/B/B/B B/A/B/B

Administration of modulators to a cell, tissue or organism may follow general protocols for the administration of agents for the treatment of the following diseases or conditions, taking into account the toxicity, if any: diabetes, diabetes complications, toxic shock, allergy, asthma, anaphylaxis, hyperglycemia-induced atherosclerosis, cataractogenesis, neuropathy, nephropathy, retinopathy, vasculopathy, an open wound, inflammation, restenosis, artery or vein graft rejection, complications from or with wound healing, microvaculopathy, macroangiopathy, heart disease, stroke, ischemia, septicemia, ischemic damage, arteriosclerosis, or stress. It is expected that the treatment cycles would be repeated as necessary. In particular embodiments, it is contemplated that various additional agents may be applied in any combination with the present invention.

In such combinations, AR inhibitors and other active agents may be administered together or separately. In addition, the administration of one agent may be prior to, concurrent to, or subsequent to the administration of other agent(s).

### EXAMPLES

The following examples are included to demonstrate particular embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Aldose Reductase Mediates the Mitogenic Signals of Cytokines

Materials and Methods

Materials: Dulbecco's Modified Eagle's Medium (DMEM), Phosphate buffered saline (PBS), penicillin/streptomycin solution, trypsin and fetal bovine serum (FBS) were purchased from GIBCO BRL Life Technologies (Grand Island, N.Y.). Antibodies against IκB-α and p65 were obtained from Santa Cruz Biotechnology. Phospho-IκB-α (Ser³²) antibody was purchased from New England BioLabs. Mouse anti-rabbit GAPDH antibodies were obtained from Research Diagnostics Inc., and anti-AR polyclonal antibodies against recombinant AR were raised in rabbits. LipofectAMINE Plus and Opti-minimal essential medium were obtained from Life Technologies, Inc. Aldose reductase antisense oligonucleotide (5′-CCTGGGCGCAGTCAATGTGG-3′) and mismatched control (scrambled) oligonucleotide (5-GGTGATAGCTGACGCGGTCC-3′) were used for transfection in VSMC to prevent the translation of AR mRNA. Consensus oligonucleotides for NF-κB (5′-AGTTGAGGGGACTTTCCCAGGC-3′) and API (5′-CGCTTGATGAGTCAGCCGGAA-3′) transcription factors were obtained from Promega Corp. Sorbinil and tolrestat were gifts from Pfizer and Ayerest, respectively. Mouse NF-κB monoclonal antibodies against p65 subunit that selectively binds to the activated form of NF-κB were obtained from Chemicon International Inc. Phorbol 12-myristate 13-acetate (PMA), 3-(4,5-dimethylthiazol-2-yl)-2,5-diphenyl tetrazolium bromide (MTT), and other reagents used in the EMSA and western blot analysis were obtained from Sigma. All other reagents used were of analytical grade.

Immunohistochemistry of balloon-injured rat carotid arteries: The carotid arteries of adult male Sprague-Dawley rats were injured as described previously (Ruef et al., 2000). Briefly, the rats were anesthetized by an intraperitoneal injection of ketamine (2 mg/kg) and xylazine (4 mg/kg). The left carotid artery was injured by balloon withdrawal 3 times, thus creating a denuded area. The right carotid artery was left uninjured and served as a control for each animal. Starting 1 day before injury and throughout the observation time, the animals were fed either the AR inhibitor—tolrestat (10 mg/kg/day) or PBS. There were no signs of toxicity related to drug exposure. Ten days after injury, the arteries were perfusion-fixed with 4% paraformaldehyde and stored in 70% ethanol. Five micron sections of formalin fixed, (fixation limited to 18 hours and tissues held in 70% alcohol until processed) paraffin embedded tissues taken from rat aorta, were stained with mouse monoclonal antibodies against activated RelA (p65) subunit of NF-κB from Chemicon (MAB 3026). Following deparaffinization and hydration, the sections were placed in a pressure cooker in Target Retrieval Solution (Dako Cat # S1699) consisting of a citrate buffer (pH 6.0) for 27½ minutes. Slides were cooled rapidly and immunostained using the Dako Autostainer. The slides were washed in Tris buffer (Dako Cat # S1968), endogenous peroxidase was removed with 3% hydrogen peroxide. The slides were incubated in primary antibody, anti-NF-κB diluted at 1:100 (10 μg of the primary antibody) for 120 min. Slides were incubated in the detection system, (Dako Cat # K0609), link and label each for 20 minutes. Slides were then incubated in the chromogen-diaminobenzidine (Dako Cat # K3466) for 10 min. Nuclei were stained in Mayer's hematoxylin at ½ the strength. Areas of positive reactivity are stained brown.

Cell culture: Rat VSMC were isolated from healthy rat aorta and characterized by smooth muscle cell specific α-actin expression. VSMC were maintained and grown in DMEM supplemented with 10% FBS and 1% penicillin/streptomycin at 37° C. in a humidified atmosphere of 5% CO₂.

Measurement of cell growth: For measuring growth, the VSMC, grown to 60 to 80% confluency, were incubated for 48 h in DMEM containing 0.1% FBS to induce quiescence. After serum-starvation for 24 h, the cells were stimulated with TNF-α (2 nM), in the presence or the absence of AR inhibitors (10 μM). Proliferation was determined by cell counts or by the MTT assay. DNA synthesis was measured by thymidine incorporation. For these experiments, [³H]-thymidine (10 μCi/ml) was added to the cells 6 h prior to the end of the serum-starvation period. Cells were harvested on Millipore multiscreen system 96-well filtration plates and were washed with PBS using multiscreen separation systems vacuum manifold. Filters were air-dried and the radioactivity was measured using a Beckman Counter, LS1801.

Cytotoxicity assays: The rat VSMC were grown in DMEM and were harvested by trypsinization and plated in a 96-well plate at a density of 2,500 or 5,000 cells/well. Cells were grown 24 h in the indicated media and were growth-arrested at 60 to 80% confluency for 24 h in media containing 0.1% FBS. Low serum levels were maintained during growth arrest to prevent slow apoptosis that accompanies complete serum deprivation of these cells. The growth-arrested cells were treated with TNF-α (10 μM to 10,000 μM), or AR inhibitors (0.5 [M to 20 μM), or medium containing both TNF-αα and AR inhibitors for another 24 h. The rate of cell proliferation or apoptosis was determined by cell count, MTT assay or the incorporation of [³H]-thymidine.

Cell number: The loss of membrane integrity indicated by the inability of the cells to exclude trypan-blue was used to measure cell viability using a hemocytometer. Briefly, the cells were harvested by trypsinization, washed and suspended in PBS, and incubated with equal amount of 0.1% trypan-blue. The percentage of trypan-blue positive cells was calculated and the values from 4 separate experiments for each treatment were used for statistical analysis.

MTT assay: Twenty five microliters of 5 mg/ml MTT were added to each well of the 96-well plate plated with VSMC. The plate was incubated at 37° C. for 2 h. The formazan granules generated by the live cells were dissolved in 100% DMSO and absorbance at 550 nm and 562 nm was monitored using a multiscanner ELISA autoreader. Cell viability was determined by the MTT-assay and direct cell counts. For these determinations, cells were incubated at 37° C. for 2 h with 25 μl of 5 mg/ml MTT. Apoptotic cell death was quantified using “Cell Death Detection ELISA” kit (Roche Inc.) as per the manufacturer's instructions. The activity of caspase-3 was measured by using the specific caspase-3 substrate Z-DEVD-AFC, (CBZ-Asp-Glu-Val-Asp-AFC) which was incubated with cell lysate and the fluorescence (ex 400 nm, em 505 nm) released by the cleavage of substrate was measured by using fluorescence 96-well plate reader.

Thymidine-incorporation: [³H]-thymidine (10 μCi/ml) was added to the cells 6 h prior to the end of the growth-arrest protocol. After mitogenic stimulation, the cells were harvested on Millipore multiscreen system, 96-well filtration plates and were washed with PBS using multiscreen separation systems vacuum manifold. Filters were air-dried and the radioactivity was measured using a LS1801 Beckman counter.

Apoptosis: Cell death was assessed by using “Cell Death Detection ELISA” kit (Roche Inc.) that measures cytoplasmic DNA-histone complexes, generated during apoptotic DNA fragmentation, and cell death detection was performed according to the manufacturer's instructions and monitored spectrophotometrically at 405 nm.

Caspase-3 activity: The activity of caspase-3 was measured by using the specific caspase-3 substrate Z-DEVD-AFC, (CBZ-Asp-Glu-Val-Asp-AFC), which was incubated with cell lysate and the fluorescence (excitation: 400 nm, emission: 505 nm) released by the cleavage of substrate was measured by using fluorescence 96-well plate reader.

Electrophoretic mobility gel shift assays (EMSA): Cytosolic and nuclear extracts were prepared as described (Chaturvedi et al., 2000). Consensus oligonucleotide for NF-κB transcription factors was 5′-end labeled using T4 polynucleotide kinase. The assay procedure was as described before (Chaturvedi et al., 2000). Briefly, nuclear extracts prepared from various control and treated cells were incubated with the labeled oligonucleotide for NF-κB for 15 min at 37° C., and the DNA-protein complex formed was resolved on 6.5% native polyacrylamide gels. The specificity of binding was examined by competition with excess of unlabeled oligonucleotide. Supershift assay was also performed to determine the specificity of NF-κB binding to its specific consensus sequence by using anti-p65 antibodies. After electrophoresis, the gels were dried by using a vacuum gel dryer and were autoradiographed on Kodak X-ray films. The radiolabeled bands were quantified by an Alpha Imager 2000 Scanning Densitometer equipped with the AlphaEase™ Version 3.3b software.

Immunostaining of VSMC with p65 antibodies: The VSMC preincubated without or with ARI for 24 h were exposed to or TNF-α (0.1 nM, 1 h) prior to immunofluorescence studies. The VSMC were fixed in 100% ice-cold acetone for 5 min and washed with PBS. Blocking was carried out in 10% goat serum in PBS for 30 min. Primary antibodies against p65 were added and incubated overnight at 4° C. Following washing with PBS, the cells were incubated with respective Alexa-488 secondary antibodies in 10% goat serum for 1 h at room temperature in the dark. The cells were washed with PBS, mounted on slides and a drop of FLUORSAVE™ reagent was added. The fluorescence staining was evaluated using Nikon Eclipse E800 epifluorescence microscope equipped with digital camera, interfaced to a computer.

Western blot analysis: Equal amount of either cytoplasmic or nuclear extracts were separated by 10% sodium dodecyl sulfate polyacrylamide gel electrophoresis (SDS-PAGE). After electrophoresis, the proteins were electroblotted to nitrocellulose filters and probed with rabbit polyclonal antibodies against either IκB-α or IκB-α-phosphorylated at Ser-32 or p65. The antibody binding was detected by enhanced chemiluminescence (Amersham Pharmacia Biotech, NJ).

Protein kinase C assay: The VSMC pretreated for 24 h with or without AR inhibitors were incubated with TNF-α (2 nM) for another 24 h. The VSMC, with or without mitogenic stimulation were washed twice with an ice-cold PBS, and sonicated with three 10-second bursts in 1 ml of the extraction buffer (25 mM Tris-HCl, pH 7.5 containing 0.5 mM EDTA, 0.5 mM EGTA, 0.05% Triton X-100, 10 mM 2-mercaptoethanol, 1 g/ml leupeptin, 1 μg/ml aprotinin and 0.5 mM phenylmethylsulfonyl fluoride). The homogenates were centrifuged at 100,000 g for 60 min at 4° C. in a Beckman ultracentrifuge. The pellets containing the membrane fraction were solublized by suspending in the assay buffer containing 1% Triton X-100 and stirring at 4° C. for 1 h. The PKC activity was measured by using the Promega Signa TECT PKC assay system. Aliquots of the reaction (25 mM Tris-HCl pH 7.5, 1.6 mg/ml phosphatidylserine, 0.16 mg/ml diacylglyceral, and 50 mM MgCl₂) were mixed with [c ³²P] ATP (3,000 Ci/mmol, 10 μCi/μl) and incubated at 30° C. for 10 min. To stop the reaction, 7.5 M guanidine hydrochloride was added and the phosphorylated peptide was separated on binding paper. After the paper was washed, the extent of phosphorylation was detected by determining the radioactivity. The incorporation of radioactivity was linear for 15 min, and the PKC activity was determined by subtracting the initial rate of protein kinase activity (in the absence of activators) from the rate of protein kinase activity in the presence of phosphatidylserine, and diacylglycerol.

Antisense Ablation of AR: VSMC grown to 60-70% confluency in DMEM supplemented with 10% FBS were washed with opti-minimal essential medium for four times, 60 min before the transfection with oligonucleotides. The cells were incubated with 1 μM AR antisense or scrambled control oligonucleotides using LipofectAMINE Plus (15 μg/ml) as the transfection reagent as suggested by the supplier. After 12 h, the medium was replaced with fresh DMEM (containing 10% FBS) for another 12 h followed by 24 h of incubation in serum free-DMEM (0.1% FBS) before TNF-α stimulation. Changes in the expression of AR were estimated by Western blot analysis using anti-AR antibodies and by measuring the AR activity in the total cell lysate.

Results

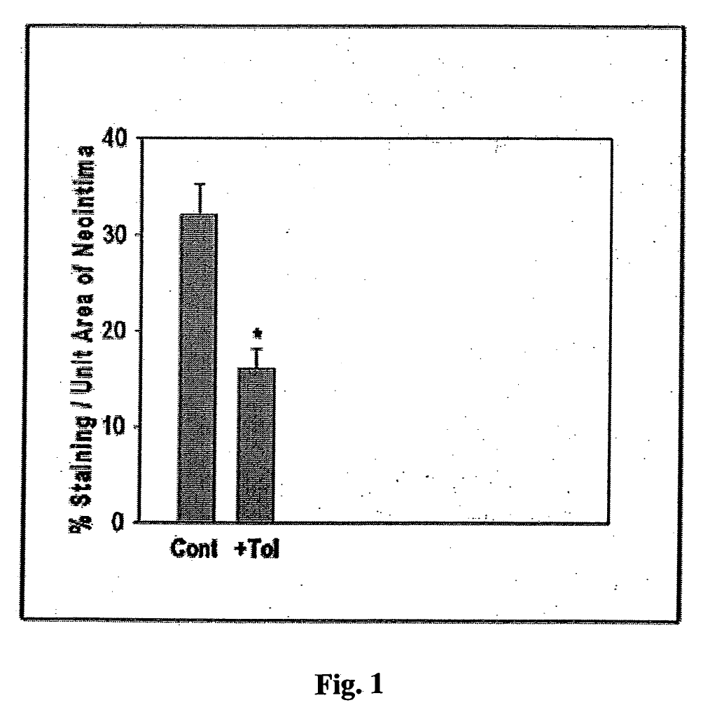

Inhibition of AR diminishes NF-κB activation: The inventors have previously reported that inhibition of AR prevents serum-induced VSMC growth in culture and decreases neointima formation in balloon-injured carotid arteries (Ruef et al., 2000). However, the mechanism by which AR facilitates VSMC growth was not examined. Because the transcription factor NF-κB plays a central role in VSMC mitogenesis (Hoshi et al., 2000; Selzman et al., 1999; Wang et al., 2001) and activated NF-κB has been localized to atherosclerotic lesions and restenotic vessels (Hajira et al., 2000), the inventors examined the effect of AR inhibition on NF-κB activity in balloon-injured arteries. Rat carotid arteries were injured as described before and were stained with antibodies that specifically recognize activated NF-κB. As shown in FIG. 1, no significant staining by antibodies directed against activated NF-κB was observed in control, uninjured carotid arteries. However, arteries obtained after 10 days of balloon injury displayed intense staining, and the intensity of staining was significantly lower in the arteries of rats fed tolrestat, indicating that inhibition of NF-κB activation could be one of the mechanism by which AR inhibitors diminish neointimal hyperplasia. To further assess the significance of this finding and to delineate the processes in mitogenic signaling sensitive to AR inhibition, The inventors examined the antimitogenic effects of AR inhibitors with VSMC in culture. For these experiments The inventors tested the effects of AR inhibition on TNF-α-mediated VSMC growth, because cell growth in injured vessels has been shown to be to a large extent due to TNF-α (Rectenwald et al., 2000; Niemann-Jonsson et al., 2001).

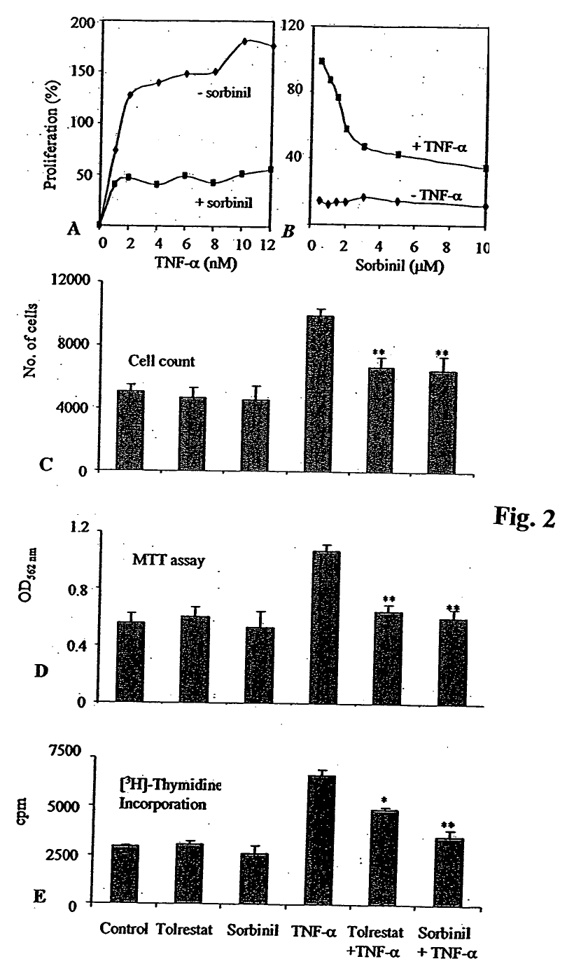

Attenuation of TNF-α-induced VSMC proliferation: To investigate the role of AR in the signal transduction pathway of TNF-α leading to VSMC proliferation, the inventors determined the effect of ARI, sorbinil or tolrestat. The extent of VSMC proliferation was determined by following VSMC cell number, MTT assay and DNA synthesis by following thymidine incorporation. The results shown in FIG. 2A demonstrate that the treatment of VSMC with several concentrations of TNF-α ranging from 1 to 12 pM for 24 h significantly stimulated VSMC growth. The increase in growth was attenuated by 10 μM sorbinil added to the incubation media under identical conditions (FIG. 2B). In the absence of TNF-α, increasing concentrations of sorbinil (from 0.1-10 μM) did not affect the growth, indicating that sorbinil by itself does not affect VSMC growth at the concentrations used (FIG. 2B). Similar results were obtained when the proliferation was estimated by counting cell number or by the MTT assay (data not shown). To rule out inhibitor-specific effects, The inventors also examined the effect of tolrestat, which is structurally different from sorbinil. Like sorbinil, tolrestat also inhibited VSMC proliferation caused by TNF-α (FIGS. 2C-E), but by itself had no effect on cell growth. Thus, inhibition of AR by two structurally-unrelated inhibitors prevents VSMC growth suggesting that AR is an obligatory mediator of TNF-α-induced VSMC growth.

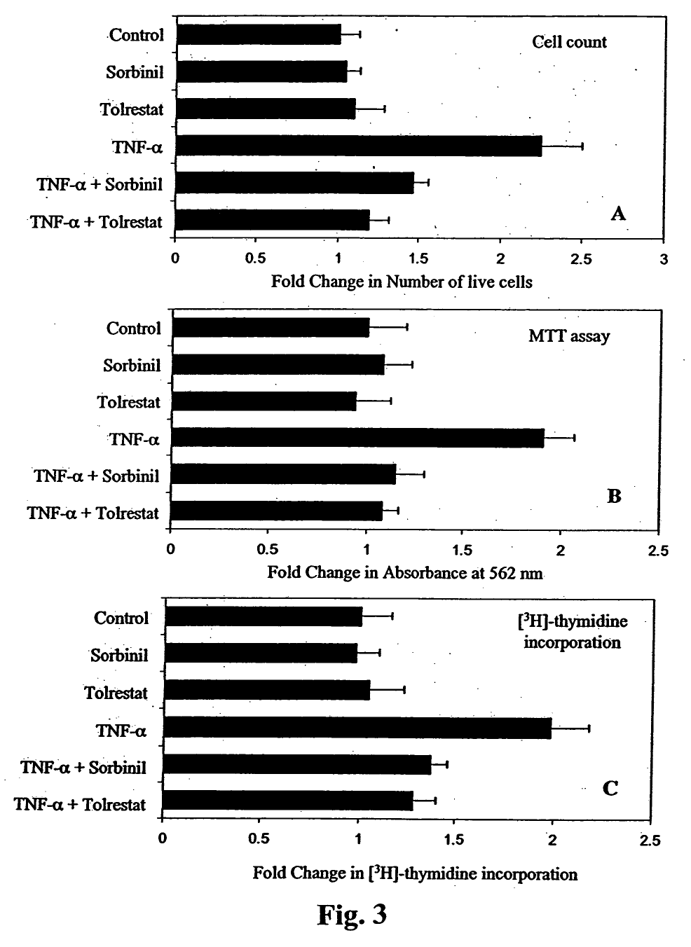

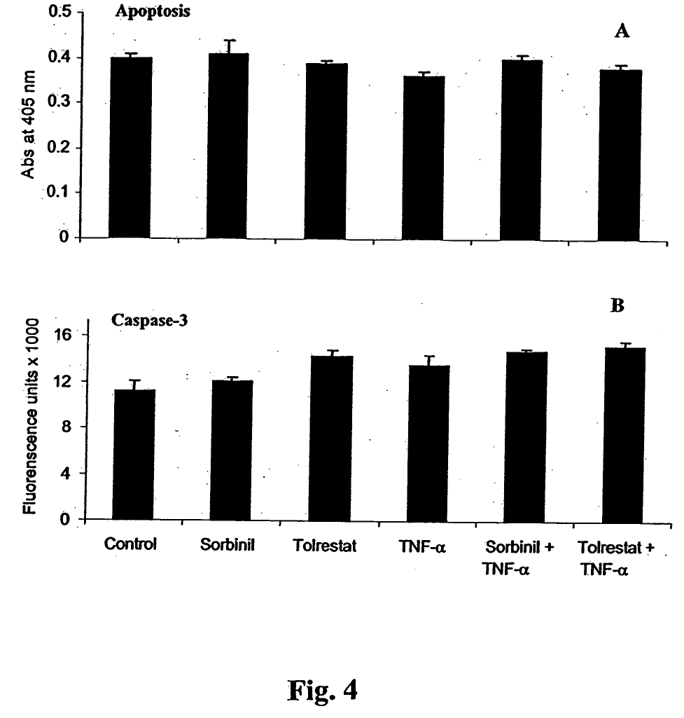

The inventors further observed that stimulation of VSMC for 24 h with TNF-α resulted in increased cell proliferation compared to non-stimulated cells (FIG. 3) as measured by cell counts using Trypan blue, thymidine incorporation, and MTT assay. Incubation of VSMC for 24 h with 10-20 μM sorbinil or tolrestat prior to stimulation with TNF-α prevented VSMC proliferation. In the absence of TNF-α, ARI did not affect VSMC growth. Together, these data suggest that inhibition of AR prevents TNF-α-induced VSMC growth, indicating that AR may be essential for the mitogenic effects of TNF-α. The ARI-mediated attenuation of TNF-α-induced VSMC proliferation is not due to apoptosis, since ARI, TNF-α or ARI+TNFα did not cause apoptosis or activation of caspase-3 (FIG. 4A and FIG. 4B).

Attenuation of VSMC proliferation by inhibiting AR is not due to apoptosis: To demonstrate that the sorbinil or tolrestat-mediated attenuation of TNF-α-induced VSMC proliferation is not due to apoptosis, the inventors measured apoptosis as well as caspase-3 activity under identical conditions used to prevent TNF-α-induced VSMC proliferation by sorbinil or tolrestat. However, neither of these inhibitors caused apoptosis or the activation of caspase-3 (data not shown), indicating that inhibition of AR prevents cell proliferation, not by increasing cell death but by inhibiting VSMC growth.

Attenuation of TNF-α-induced activation of NF-κB: The inventors next examined whether in cultured VSMC, inhibition of AR prevents TNF-α-mediated activation of NF-κB as observed in restenotic vessels (FIG. 1). Upon stimulation of VSMC with TNF-α, a pronounced activation of NF-κB was observed as determined by EMSA. To examine the role of AR, the inventors preincubated the VSMC for 24 h with different concentrations of sorbinil followed by incubation with TNF-α (0.1 nM) for 60 min at 37° C. and determined NF-κB activity by EMSA. To ascertain that the gel-retarded band, observed with the TNF-α-treated cells was indeed due to NF-κB, the inventors incubated the nuclear extract from TNF-α-activated cells with antibodies to p65 subunit followed by NF-κB determination by EMSA. Antibodies to p65 shifted the band to a higher molecular weight, at the same time, the preimmune serum had no effect on the mobility of NF-κB. In addition, excess (20 and 50 fold) cold NF-κB oligonucleotide completely eliminated the band, indicating that it was specifically due to NF-κB. These observations validate the measurement of NF-κB activity and substantiate that the specific activity reported by EMSA is entirely due to NF-κB activation. However, almost 60% of the TNF-α-induced NF-κB activation was prevented by 10 μM sorbinil. The extent of inhibition by sorbinil was dose-dependent, although sorbinil by itself did not activate NF-κB even when added to a concentration of 100 μM. On the basis of these observations The inventors conclude that inhibition of AR prevents TNF-α-induced activation of NF-κB.

To examine the mechanisms of inhibition of NF-κB, the inventors tested whether the effect of sorbinil could be overcome by higher concentration of TNF-α. Sorbinil (10 μM)-pretreated or -untreated VSMC were incubated with various concentrations of TNF-α (0-10,000 pM) for 60 min, and the activation of NF-κB was measured. Although, compared to 0.1 nM, 10 nM TNF-α caused a more pronounced activation of NF-κB, the extent of inhibition by sorbinil was unaffected by the concentration of TNF-α. To determine the minimum duration of sorbinil exposure required to prevent TNF-α signaling, VSMC were incubated with 10 μM sorbinil for 0-48 h prior to stimulation by TNF-α for 60 min. A significant inhibition of TNF-α-mediated activation of NF-κB in cells pre-incubated with ARI for 12 h was observed. However, for maximal inhibition, 24 h pretreatment of VSMC was necessary. No significant inhibition of NF-κB activation was observed when sorbinil and TNF-α were added together for 60 min. These results demonstrate that the extent of NF-κB inhibition by sorbinil is independent of the extent to which the pathway is activated, and that the inhibition requires prolonged pre-incubation, suggesting that changes in metabolism and/or gene expression may be necessary for sorbinil to disrupt TNF-α-signaling.

In addition to TNF-α, NF-κB is also activated by a variety of stimuli including growth factors such as PDGF-AB, bFGF, and Ang-II. The inventors, therefore, tested whether inhibition of AR would also prevent activation of NF-κB caused by mitogens other than TNF-α. For this, untreated or sorbinil-treated VSMC were incubated with mitogenic concentrations of bFGF, PDGF-AB and the hypertrophic concentration of Ang-II, and the activation of NF-κB was measured by EMSA. In all instances, a pronounced increase in the activity of NF-κB was observed, and preincubation of VSMC with sorbinil led to a decreased activation of NF-κB in FGF, PDGF or Ang-II stimulated cells. At the same time inhibition of AR did not attenuate NF-κB activation induced by the phorbol ester, PMA. On the basis of these observations The inventors conclude that inhibition of AR prevents NF-κB activation, regardless of the nature of the receptor involved in the process.

Attenuation of TNF-α-induced phosphorylation and degradation of IκB-α and NF-κB nuclear translocation: Extensive investigations show that phosphorylation, ubiquitination and proteolytic degradation of IκB-α precede the activation of NF-κB in the cytosol and the active dimer of NF-κB translocates to the nucleus, where it binds to specific DNA sequences and activates the transcription of inflammatory genes (Bours et al., 2000; Jourd'heuil et al., 1997; Rath and Aggarwal, 1999). The inventors, therefore, investigated whether the inhibition of AR prevents the phosphorylation and degradation of IκB-α. The inventors determined the effect of sorbinil on the cellular abundance and phosphorylation state of IκB-α protein by Western blot analysis using antibodies against IκB-α and phospho-IκB-α. Upon stimulation of VSMC with TNF-α, a partial IκB-α phoshophorylation in the VSMC was observed within 5 min and complete phosphorylation occurred by 15 min. However, when sorbinil-pretreated VSMC were stimulated with TNF-α, little or no phosphorylation of IκB-α was observed for 120 min (maximal observation time). Because the phosphorylated IκB-α is prone to proteolytic degradation, the inventors next determined the effect of sorbinil on the degradation of IκB-α. Upon stimulation with TNF-α, a complete degradation of IκB-α was observed in 15 min and full resynthesis was achieved in 30 min. However, in sorbinil-pretreated cells, no degradation of IκB-α was observed for a total observation time of 120 min. Since transcriptional activation by NF-κB requires its nuclear translocation where it can bind to its specific consensus sequences and activate the transcription of target genes, the inventors measured NF-κB activity by EMSA in the nuclear extracts and further identified NF-κB translocation by Western blot analysis using p65 antibodies in the cytosolic and nuclear extracts, 60 min after stimulation with TNF-α. Exposure of VSMC to TNF-α for 30 min resulted in the translocation of NF-κB to the nucleus, which was maximal in 60 min. However, in the sorbinil-pretreated cells, the inventors observed only a partial translocation of NF-κB in 60 min after exposure to TNF-α. From these results it is concluded that sorbinil inhibits the TNF-α-induced phopshorylation of IκB-α, prevents its proteolytic degradation, and attenuates active p65/p50 (NF-κB) dimer translocation from cytosol to nucleus.

Incubation with TNF-α led to nuclear localization of fluorescence, which corresponded to the intracellular staining of the Hoeshst nuclear dye, indicating that TNF-α, induces nuclear localization of p65. However, when the tolrestat-pretreated cells were stimulated with TNF-α, no nuclear staining was observed and these cells continued to show diffused perinuclear staining. Thus, the inhibition of AR prevents TNF-α-induced nuclear translocation of p65.

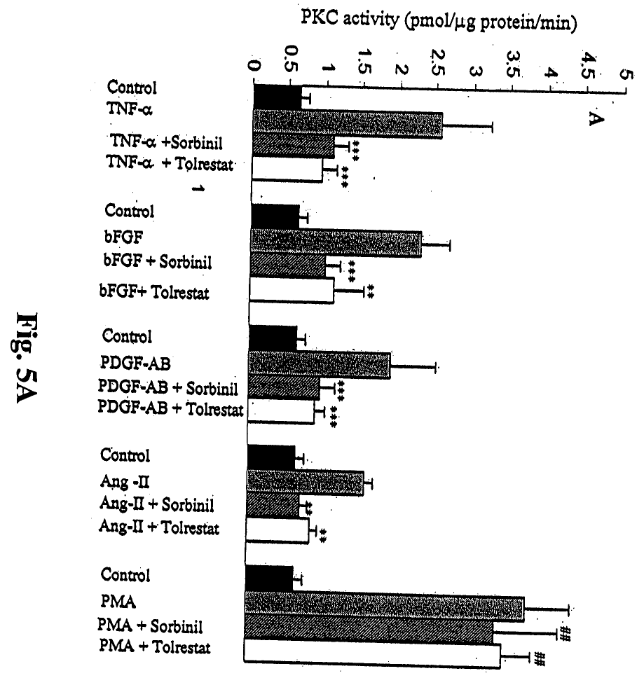

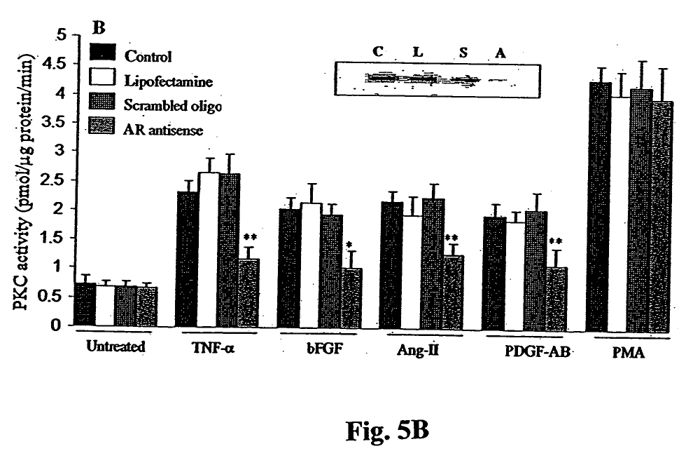

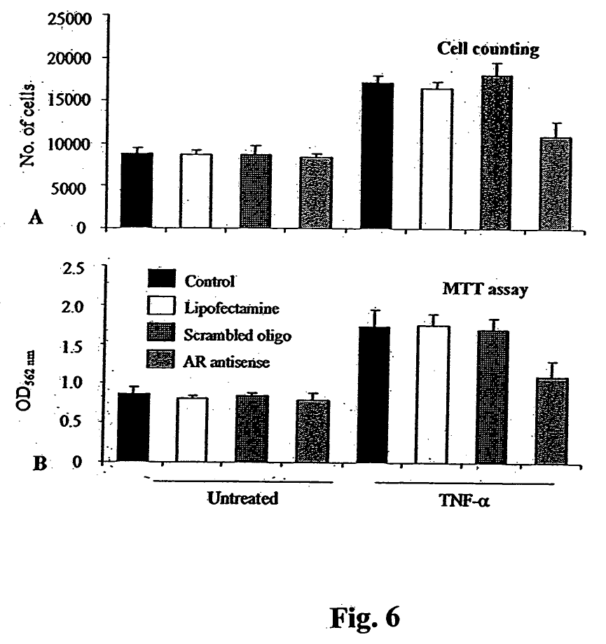

Attenuation of PKC activation: TNF-α and other VSMC mitogens are known to activate the PKC family of kinases possibly by first activating phospholipase A₂. The inventors therefore, incubated the VSMC without or with sorbinil or tolrestat for 24 h followed by the addition of TNF-α, PDGF-AB, bFGF, Ang-II and PMA. All these agents led to the activation of the total membrane bound PKC activity. The activation of PKC by all the agents except PMA was strongly abrogated by sorbinil as well as tolrestat (FIG. 5A). The PMA-induced PKC activation was not affected by inhibiting AR (FIG. 5A) under similar conditions, the activation of cytosolic PKC was not affected by the AR inhibitors themselves. Although The inventors used two structurally-unrelated compounds that selectively inhibit AR (Bhatnagar et al., (1990); Rittner et al., 1999), the non-specific effects of these drugs could not be rigorously excluded. Therefore, the inventors transfected VSMC with antisense AR oligonucleotides that decreased AR protein expression by >80% (FIG. 5B inset) and also the enzyme activity. In contrast to the cells transfected with scrambled oligonucleotides, cells transfected with antisense AR displayed markedly attenuated activation of PKC upon stimulation with TNF-α, bFGF, PDGF-AB or Ang-II (FIG. 5B), indicating that similar to pharmacological inhibition, antisense ablation of AR prevents PKC activation. Moreover, consistent with the pharmacological data, transfection with antisense, but not scrambled oligonucleotides, attenuated TNF-α-induced proliferation as assessed by cell count and MTT assay (FIG. 6). Together, these observations suggest that the anti-mitogenic effects of tolrestat and sorbinil are not a reflection of their non-specific toxicity, but are specific to the inhibition of AR and that reaction product(s) of AR catalysis may be involved in this signaling process.

AR inhibitors are specific to redox-sensitive transcription factors: Because activating NF-κB, TNF-α is known to activate the transcription factor-AP1, the inventors determined the effect of sorbinil on the TNF-α-induced activation of AP1. The VSMC were preincubated for 24 h with different concentrations of sorbinil, after which the cells were stimulated with TNFα (0.1 nM) for 60 min at 37° C. and AP1 activity was determined by EMSA. Pretreatment with 10 μM sorbinil caused a 60% decrease in the TNFα-induced activation of AP1. To determine the specificity of ARI towards non-redox sensitive transcription factors, we investigated the effect of ARI on constitutive transcription factors such as SP1 and OCT1. ARI alone or in combination with TNF-α had no effect on the modulation of these transcription factors indicating the specificity of ARI towards redox-insensitive transcription factors.

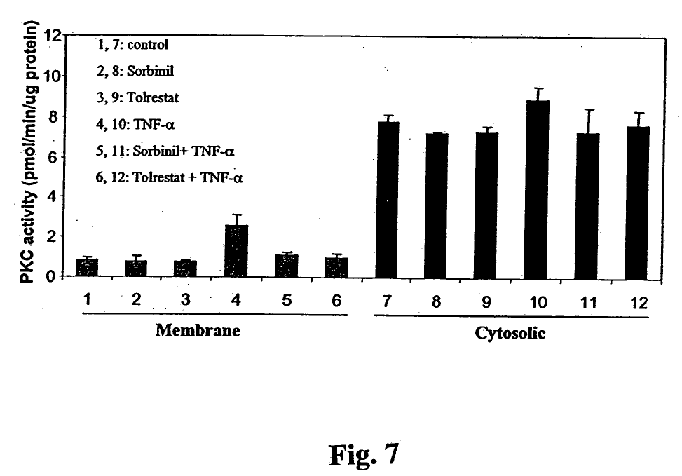

The cytokine TNF-α is known to activate PKC possibly by first activating phospholipase A₂. We therefore, incubated the VSMC without or with ARI for 24 h followed by the addition of TNF-α. We observed that the TNF-α-induced activation of membrane bound but not cytosolic PKC was drastically inhibited by ARI (FIG. 7). Although, the inventors did not identify the specific PKC isoform activated, the diacylglycerol (DAG) and Ca²⁺ activated PKC isozymes appears to be the most likely candidates since TNF-α-induced activation of phospholipase is known to activate DAG and IP₃. Finally, our results show that phorbal ester-induced activation of PKC as well as NF-κB was not affected by ARI (data not shown).

### Example 2

### Nitric Oxide Regulates the Polyol Pathway of Glucose Metabolism in Vascular Smooth Muscle Cells

Materials and Method

Materials: S-Nitroso-N-acetylpenicillamine (SNAP), diethylamine NONOate (NONOate), S-nitrosoglutathione mono-ethyl-ester (GSNO-Ester), [2-(4-carboxyphenyl)-4,4,5,5-tetramethylimidazoline-1-oxyl-3oxide] (carboxy-PTIO), L-arginine and N^{G}-nitro-L-arginine methyl ester (L-NAME) were purchased from Calbiochem. S-nitrosoglutathione (GSNO), 3-morpholinosydnonimine (SIN-1), NADPH, D,L-glyceraldehyde, D,L-dithiothreitol (DTT), cycloheximide and protease inhibitor cocktail (AEBSF, Leupeptin, Bestatin, E-64, Pepstatin-A) were obtained from Sigma. Sorbinil and tolrestat were obtained as gifts from Pfizer and Ayrest, respectively. Deriva-Sil was purchased from Regis Technologies Inc., USA. Polyclonal antibodies against recombinant AR were raised in rabbits. [³⁵S]-L-cysteine was obtained from New England Nuclear. Dulbecco's modified Eagle's medium (DMEM), phosphate-buffered saline (PBS), penicillin/streptomycin solution, trypsin and fetal bovine serum (FBS) were purchased from GIBCO BRL Life Technologies. Reagents for sodium dodecyl sulfate polyacrylamide gel electrophoresis (SDS-PAGE) and transblotting were obtained from Bio-Rad. All other reagents were of analytical grade.

In vivo regulation of polyol pathway in normal and diabetic rat aorta: To investigate the in vivo effects of NO, diabetes was induced in ˜3 months old Sprague-Dawley rats by injecting streptozotocin (STZ; 65 mg/kg body wt). Only those rats, which had, blood glucose levels >400 mg % on the 4^{th day} of the STZ injection were used in the study (group II). Non-diabetic and diabetic rats were divided in four groups each-groups I to IV nondiabetic and groups V-VIII diabetic. Groups I and V were injected with the carrier; groups II and IV with L-arginine (200 mg/kg body wt); groups III and VII with L-NAME (50 mg/kg body wt); and in groups IV and VIII nitroglycerine patches were applied which released 200 ng NO/min. The nitroglycerine patches were applied to the pre-shaved dorsal neck region, and were replaced every day. After 10 days of treatment, the rats were euthanized and their aorta was removed. The aorta was homogenized in 1 ml of PBS containing 20 μl of the protease inhibitor cocktail. The AR activity and sorbitol content of the homogenates were measured. Data is presented as mean±SEM and the P values were determined by unpaired students t-test using Microsoft Excel 2000.

Regulation of AR activity and sorbitol accumulation in aorta ex vivo: The abdominal aorta was dissected from Sprague-Dawley rats, C57/BL-6 mice, or the eNOS-null mice in the C57/BL6 background (obtained from Jackson Laboratories). The aorta was dissected into six 5 mm strips. Aortic strips from 6 to 8 animals were pooled and divided into groups with 6 random pieces in each group. The aortic strips were incubated in M-199 medium containing 10% fetal bovine serum, 1% penicillin/streptomycin and 2 μg/ml cycloheximide in the absence or presence of 2 mM L-arginine or 1 mM L-NAME at 37° C. in a humidified CO₂ incubator. After 12 h of incubation, 50 mM glucose was added to the medium and the incubation was continued for another 24 h. The samples were washed with ice cold PBS and homogenized in 1 ml of 0.1 M phosphate (pH 7.4) containing protease inhibitor cocktail, and the AR activity and the sorbitol content were measured (Ramana et al., 2000; Dixit et al, 2000).

Cell culture and treatment: The VSMC were maintained and grown to confluency in DMEM supplemented with 10% FBS and 1% penicillin/streptomycin at 37° C. in a humidified atmosphere of 5% CO₂. Prior to the addition of the NO-donors, the medium was replaced with Krebs-Hansliet (KH) buffer containing (in mM): NaCl, 118; KCl, 4.7; MgCl₂, 1.25; CaCl₂, 3.0; KH₂PO₄, 1.25; EDTA, 0.5; NaHCO₃, 25; glucose 5, pH 7.4. Freshly prepared solutions of the nitric oxide donors (SNAP, SIN-1, GSNO, GSNO-ester, NOC-9 or NONOate) or AR inhibitors (sorbinil and tolrestat) at a final concentration of 1 mM were added to the culture medium. In some experiments, SNAP was added to the VSMC cultured in the presence of DMEM with 10% FBS. The samples were incubated at 37° C. under 5% CO₂ for 2 h, after which 40 mM glucose was added to the incubation medium and the incubation was continued for an additional 4 h. For regeneration of the AR activity, the VSMC were incubated with NO-donors for 2 h followed by the replacement of the media with fresh media without NO-donors and the incubation was continued for an additional 6 h. The cells were harvested and lysed in 10 mM phosphate (pH 7.0) containing 20 μl of the protease inhibitor cocktail. An aliquot of the sample was removed to determine the total protein content and AR enzyme activity and the rest of the sample was used to measure sorbitol.

Measurement of AR and sorbitol: Tissues or cells were homogenized in 1 ml of 0.1 M phosphate (pH 7.4) containing protease inhibitor cocktail. The AR activity was measured using glyceraldehyde as substrate as described previously (Ramana et al., 2000; Dixit et al., 2000). For sorbitol measurements, proteins in the homogenate (0.5 ml) were removed by precipitating with Ba(OH)₂ and ZnSO₄ (0.5 M each). The 10,000×g supernatants were ultrafiltered using Amicon YM-10 microcon and lyophilized. The lyophilized samples were dried overnight in a vacuum desiccator over CaCl₂ and derivatized by adding 0.1 ml of Deriva-Sil. One microliter of the derivatized sample was applied to a Varian 3400 gas chromatograph coupled to a hydrogen flame ionization detector. The sugars were separated on a Chrompack capillary column packed with CP Sil 24CB. The column temperature was set at 140° C. and programmed to increase at a rate of 4° C./min to 170° C. then to 250° C. at a rate of 50° C./min. The temperature was then held constant for an additional 3 min. The injection port was maintained at 250° C. and detector temperature was set at 300° C. The amount of sorbitol in the sample was calculated using reagent sorbitol derivatized and processed using an identical protocol.

Metabolic labeling of VSMC and immunoprecipitation of AR: The medium from the flask containing confluent VSMC was removed and the cells were washed with the KH buffer. The cells were then re-incubated with the KH buffer containing 2 μg/ml of cycloheximide (to inhibit protein synthesis) at 37° C. in 5% CO₂. After 60 min of incubation, 20 μmol/ml L-[³⁵S]-cysteine was added to the flask and the cells were incubated for an additional 5 h to label the glutathione pool. The metabolically-labeled cells were incubated with SNAP for the indicated durations. To immunoprecipitate AR, the cells were lysed with cold Tris-Triton buffer (1% Triton X-100, 150 mM NaCl, 10 mM Tris pH 7.4, 1 mM EDTA, 1 mM EGTA, 0.2 mM Na₂O₂V₇, 0.2 mM PMSF, 0.5% NP-40 and 20 μl of protease inhibitor cocktail) and centrifuged at 10,000×g for 5 min at 4° C. An aliquot of the supernatant was used for measuring the protein concentration. To 500 μg of total lysate protein, 2 volumes of immunoprecipitation buffer (2% Triton X-100, 300 mM NaCl, 20 mM Tris pH 7.4, 2 mM EDTA, 2 mM EGTA, 0.4 mM Na₂O₂V₇, 0.4 mM PMSF, 1.0% NP-40 and 20 μl of protease inhibitor cocktail) and 50 μg of affinity-purified AR antibodies were added and the samples were incubated at 4° C. for 2 h. After the incubation, 100 μl of protein-A Agarose beads were added and the samples were incubated overnight on a shaker at 4° C. to precipitate free and bound IgG. The samples were centrifuged at 10,000×g for 5 min and washed twice with immunoprecipitation buffer. The pellet was resuspended in 50 μl of 250 mM Tris pH 6.8 containing 4% SDS, mixed and centrifuged at 10,000×g for 5 min. The supernatant was used for SDS-PAGE using 10% gel. The gel was then dried and autoradiographed.

Results

Regulation of the polyol pathway by NO in normal and diabetic rats: In the first series of experiments, we examined whether NO regulates the polyol pathway in situ. For this, the inventors studied both diabetic and non-diabetic rats in which NO synthesis was stimulated or inhibited. In addition, the inventors tested the possibility that exogenous NO delivery by nitroglycerine patches could affect polyol accumulation. In control, non-diabetic rats, the sorbitol content of the dorsal aorta was minimal (3.5 nmoles/mg protein). However, this was considerably higher in the aorta of diabetic rats (Table 1). The dramatic 22-fold difference in the sorbitol content of the diabetic and non-diabetic aorta was correlated with a 20-fold higher AR activity in the homogenates of aorta from diabetic rats as comparted to aorta from non-diabetic rats. These results demonstrate that diabetes is associated with a marked upregulation of the polyol pathway, which could be accounted for by a parallel increase in AR activity, and that the diabetic changes in the pathway lead to a net accumulation of sorbitol in the vessel wall.

TABLE 1
Regulation of AR activity and sorbitol accumulation by
NO in non-diabetic and diabetic rat aorta.

| | Non-Diabetic Rats | | Diabetic Rats | |
|---|---|---|---|---|
| | | Sorbitol | | Sorbitol |
| | AR | content | AR activity | content |
| | activity (mU/ | (nmoles/ | (mU/mg | (nmoles/mg |
| Treatment | mg protein) | mg protein) | protein) | protein) |
| Vehicle | 6.7 ± 9.95 | 3.5 ± 0.46 | 145.7 ± | 83.8 ± 5.1 |
| | | | 11.13 | |
| L-NAME | 11.8 ± 0.65* | 6.2 ± 0.77* | 245.2 ± | 211.6 ± 26.3** |
| | | | 29.3** | |
| L-arginine | 4.4 ± 0.35* | 2.7 ± 0.40* | 54.6 ± | 14.8 ± 1.9** |
| Nitro- | 5.6 ± 1.49* | 2.9 ± 0.48* | 74.7 ± | 436 ± 25** |
| glycerine | | | 10.0** | |
| patch | | | | |

Male Sprague-Dawley rats were made diabetic by a single intraperitoneal injection of streptozotocin (65 mg/kg body wt). Both normal and diabetic rats were injected with L-arginine (200 mg/kg body wt/day) or L-NAME (50 mg/kg body wt/day). Nitroglycerine patches were applied on the pre-shaved dorsal neck region of the rats. At the end of the experiment, the aorta was removed and homogenized and the AR activity and sorbitol content of the homogenates were measured as described under Experimental Procedures. Data represents mean±S.E. (n=5) **P<0.001, * P<0.01, as compared to the vehicle-treated group.

To examine whether NO affects the vascular activity of the polyol pathway, non-diabetic and diabetic rats were treated with L-arginine, a substrate of nitric oxide synthase (NOS) which when delivered systemically increases NO production. As shown in Table 1, the L-arginine-treated rats accumulated 25% less sorbitol in the aorta as compared to untreated animals. The inhibitory effects were more pronounced in diabetic rats, in sorbitol content of the aorta was 80% lower as compared to the untreated animals. The decrease in sorbitol accumulation in diabetic and non-diabetic aorta upon L-arginine treatment was accompanied by a corresponding inhibition of AR activity. Application of the nitroglycerine patches also resulted in decreased levels of sorbitol and AR activity in the diabetic and non-diabetic aorta. However, sorbitol levels and AR activity decreased less dramatically than that observed with L-arginine (Table 1). Collectively, these observations indicate that NO inhibits AR and polyol accumulation in the aorta of diabetic and non-diabetic rats. To test the converse case, i.e., inhibition of NO synthesis promotes sorbitol accumulation, the inventors examined the effects of the NOS inhibitor—L-NAME. As shown in Table 1, treatment with L-NAME led to a 1.7-fold increase in sorbitol accumulation in the non-diabetic rats and a 3-fold increase in diabetic rats. These changes were accompanied by a proportionate increase in AR activity (Table 1), suggesting that inhibiting NO synthesis increases sorbitol accumulation and AR activity.

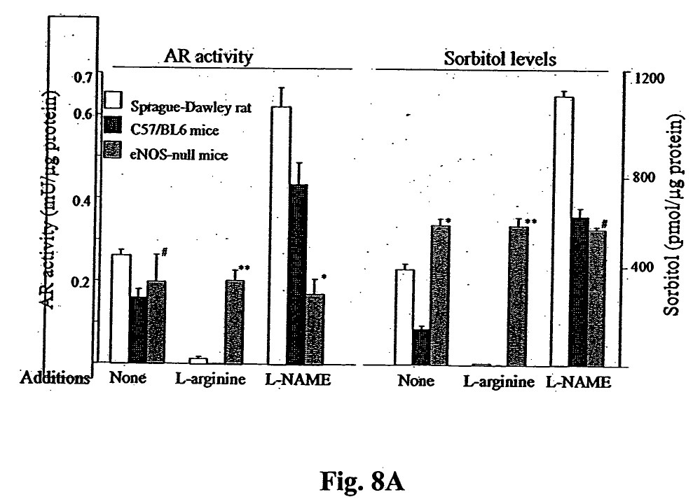

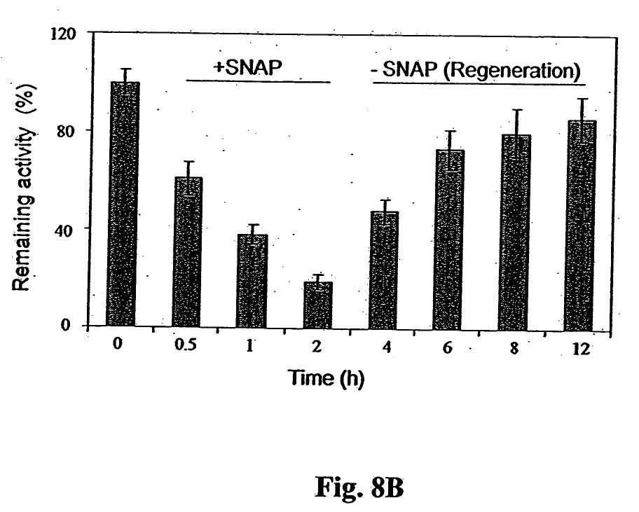

Acute regulation of AR by NO: Chronic changes in AR activity and sorbitol accumulation in the aorta of non-diabetic and diabetic animals are likely to be due to multiple processes and regulatory influences. Hence to assess whether NO could acutely affect AR activity, we examined the role of NO in regulating sorbitol accumulation in ex vivo preparations of aorta. Ex vivo changes in the polyol pathway are unlikely to be modulated by NO-induced changes in hormones and cytokines, which could influence the polyol pathway. Furthermore, to minimize the confounding influence of NO on protein expression, the incubation medium was supplemented with cycloheximide to inhibit protein synthesis. Under these conditions, incubation of the aortic strips with 50 mM glucose resulted in significant accumulation of sorbitol. The accumulation of sorbitol in the aortic strips of eNOS-deficient mice was, however, significantly greater than those prepared from the wild type (C57/BL6) mice, indicating that the lack of eNOS promotes sorbitol accumulation. Addition of L-arginine to the medium completely abolished the sorbitol accumulation and inhibited AR activity in the aortic strips prepared from non-diabetic Sprague-Dawley rats or C57/BL6 mice. However, L-arginine did not inhibit either the AR activity or sorbitol accumulation in the aortic strips of eNOS-null mice (FIG. 8A), indicating that the inhibitory effects of L-arginine are entirely due to its ability to stimulate NO synthesis via eNOS and that it does not directly influence AR activity or sorbitol formation. Similarly, inhibition of NOS by L-NAME led to a significant increase in the AR activity and sorbitol accumulation in the aortic strips prepared from Sprague-Dawley rats or C57/BL6 mice. However, L-NAME had no significant effect on either the AR activity or the sorbitol accumulation in aorta strips prepared from eNOS-null mice. Together, these data suggest that the ability of L-NAME and L-arginine to modulate the vascular activity of the polyol pathway is entirely due to their effects on eNOS and that NO-derived from the endothelium is a key modulator of polyol synthesis. Moreover, these data provide additional evidence supporting the observations made in situ that increased generation of NO leads to an increase in AR activity and sorbitol accumulation, whereas inhibition of NO generation has the opposite effect. Moreover, because the ex vivo effects were observed in the absence of protein synthesis, they raise the possibility that post-translational modification of AR may be a significant mechanism by which NO regulates the polyol pathway.

Effect of NO donors on VSMC: To probe the post-translational mechanism by which NO regulates AR, the inventors used cultured VSMC in which NO levels could be controlled readily in a homogenous cell population without using NOS inhibitors or activators. For these studies, the confluent VSMC were incubated in KH buffer with several concentrations of SNAP ranging from 0.25 to 2.0 mM for 2 h, after which the cells were harvested, lysed and used for measuring sorbitol and AR. Incubation with SNAP led to a dose-dependent decrease in AR activity (data not shown). Incubation with 1 mM SNAP led to a progressive decline in the enzyme activity and maximum (˜80%) inhibition was observed after 2 h of incubation with 1 mM SNAP (FIG. 8B). When the SNAP containing medium was removed and the cells were re-incubated in SNAP-free medium, a progressive increase in the AR activity was observed and >85% of the activity was restored, indicating that the inhibition of AR by SNAP was readily reversible.

To prevent the non-specific binding of NO to serum proteins, the studies with SNAP were conducted in serum-free KH medium. However, removal of serum could adversely affect the viability of VSMC or initiate signaling events, which could affect the regulatory role of NO. Therefore, in one series of experiments, the inventors incubated the VSMC with SNAP in DMEM containing 10% FBS. In these experiments, the AR activity was inhibited by SNAP even in the presence of the serum, although five times more SNAP (5 mM) was required to inhibit 60% of the enzyme activity in 6 h (data not shown). These observations suggest that inhibition of AR by SNAP persists in the presence of serum and is not secondary to the stress induced by serum-withdrawal. Furthermore, to ascertain that the inhibition of AR was due to NO and not restricted to SNAP, the inventors investigated the effects of other NO donors, and tested whether scavenging NO could abolish AR inhibition. As shown in Table 2, the incubation of VSMC with KH buffer containing 1.0 mM each of SNAP, GSNO, GSNO-ester, NONOate, or NOC-9 resulted in a 60 to 80% decrease in the AR activity. To examine the cellular consequences of inhibiting AR, we measured changes in the sorbitol accumulation. The sorbitol levels of VSMC incubated in medium containing 5.5 mM glucose were very low, ˜10 nmoles/mg protein. However, when the cells were incubated with 40 mM glucose for 4 h, high concentrations of sorbitol to the level of 150 nmoles/mg protein were observed. To test whether the accumulation of sorbitol by these cells was due to AR, the effects of two structurally different AR inhibitors was studied. As shown in Table 2, incubation with tolrestat or sorbinil inhibited 95 to 97% of sorbitol accumulation. These results show that the generation of sorbitol in these cells is entirely, if not exclusively, due to AR-mediated reduction of glucose. When the VSMC were incubated with the NO-donors, there was a marked decrease in cellular sorbitol content as compared to untreated cells incubated in the medium without the NO donors. The extent of inhibition of sorbitol accumulation was comparable to the extent of inhibition of AR activity. No inhibition of AR was observed with the non-NO containing analogs of these compounds (data not shown), indicating that the inhibition was specifically due to the release of NO. Furthermore, the inhibition of AR activity by SNAP was prevented by the NO scavenger PTIO, confirming that the inhibition of AR was due to NO released from SNAP and not due to non-specific effects of the donor itself. Thus, together, these series of experiments show that NO inhibits AR in VSMC in culture, and that this inhibition prevents sorbitol accumulation and is readily reversed upon removing NO.

TABLE 2
Effect of NO donors on AR activity and sorbitol levels in
VSMC incubated with 40 mM glucose

| | AR Activity | |
|---|---|---|
| Additions | (mU/μg protein) | Sorbitol level (pmol/μg protein) |
| None | 11.5 ± 0.7 | 149.8 ± 10.3 |
| SNAP | 3.3 ± 0.6**## | 16.1 ± 3.2**^{##} |
| GSNO | 3.6 ± 0.6** | 37.7 ± 1.5** |
| GSNO-Ester | 3.1 ± 0.4** | 41.2 ± 2.3** |
| NOC-9 | 4.7 ± 0.3** | 44.2 ± 7.0** |
| NONOate | 2.5 ± 1.5** | 53.6 ± 25.3** |
| Tolrestat | 2.5 ± 0.9** | 3.4 ± 4.4** |
| Sorbinil | 2.7 ± 0.5** | 6.0 ± 1.9** |
| PTIO + SNAP | 7.7 ± 1.5 | 122.0 ± 23.3 |
| *Regeneration | | |
| studies | | |
| SNAP removed | 6.9 ± 0.1 | 137.0 ± 16.3 |
| GSNO removed | 8.8 ± 0.6 | 117.3 ± 34.5 |

The AR activity in cells cultured in the 5.5 mM glucose alone was 9.0 mU/μg protein and their sorbitol content was below the detection limit. The values are the means±S.D. of three separate experiments. ***P<0.001, ** P<0.01, as compared to untreated group with NO donor treated group, and ## P<0.01 when PTIO-SNAP-treated group was compared with the SNAP-treated group.

S-thiolation of AR: The previous studies show that incubation of recombinant AR with GSNO leads to glutathiolation of the enzyme at cys-298. To examine whether NO donors S-thiolate the AR protein in VSMC, these cells were preincubated with [³⁵S] L-cysteine in the presence of the protein synthesis inhibitor, cycloheximide to prevent direct incorporation of the label in the cellular proteins, and to generate an intracellular pool of [³⁵S]-labeled GSH. After the metabolic labeling, the cells were incubated with 1 mM SNAP, and the AR protein was immunoprecipitated using anti-AR antibodies, and separated by SDS-PAGE under reducing and non-reducing conditions. Maximal labeling of the protein was achieved in 2 h, which corresponds in time to the progressive inhibition of VSMC AR upon SNAP treatment. Replacement of the incubation solution with the culture media without SNAP resulted in a significant loss of [³⁵S] label from AR in 6 h. Moreover, the radioactivity associated with AR was considerably diminished when the protein was separated on reducing gels containing □-mercaptoethanol, demonstrating that the label was incorporated in the protein via a disulfide bond. Finally, to investigate the possibility that SNAP might decrease the AR activity by suppressing the protein levels of AR, equal amounts of the immunoprecipitate were loaded on SDS-PAGE, and Western blot analysis was performed using anti-AR antibody. No changes in the AR protein levels suggests that the differences in the radioactivity associated with the AR band could not be accounted for by changes in protein expression and are specifically due to S-thiolation of AR in the SNAP-exposed cells.

### Example 3

### Regulation of Aldose Reductase and the Polyol Pathway Activity by Nitric Oxide

Materials and Methods

Material: S-Nitroso-N-acetylpenicillamine (SNAP), S-nitrosoglutathione mono-ethyl-ester (GSNO-ester), and [2-(4-carboxyphenyl)-4,4,5,5-tetramethylimidazoline-1-oxy]-3oxide] (carboxy-PTIO) were purchased from Calbiochem. S-nitrosoglutathione (GSNO), 3-morpholinosydnonimine (SIN-1), NADPH, D,L-glyceraldehyde, D,L-dithiothreitol (DTT), cycloheximide and protease inhibitor cocktail (AEBSF, Leupeptin, Bestatin, E-64, Pepstatin-A) were obtained from Sigma. Deriva-Sil was purchased from Regis Technologies Inc., USA. All other reagents were of analytical grade.

In vitro modification of aldose reductase (AR) by nitric oxide donors: Human recombinant AR was purified as described earlier (Chandra et al. (1997)). Before the start of each experiment, stored AR was reduced by incubating with 0.1 M DTT at 37° C. for 1 h and passed through a Sephadex G-25 column (PD-10). The enzyme activity was determined in a 1 ml system containing 10 mM HEPES, pH 7.4, 10 mM D,L-glyceraldehyde and 0.15 mM NADPH at 25° C. Reduced AR was incubated with various freshly prepared NO donors such as GSNO, SNAP or GlycoSNAP (1 mM each) in 0.1 M potassium phosphate, pH 7.0, at 25° C. and aliquots from the reaction mixture were withdrawn at different time intervals to measure the enzyme activity as described above. The NO-modified forms of AR were identified by electrospray ionization mass spectrometry (ESI⁺/MS) using a Micromass LCZ mass spectrometer. The desalted enzyme was diluted with the flow injection solvent consisting of 50:50:1 (v/v/v) of 10 mM ammonium acetate:acetonitrile:formic acid. The solution was introduced into the mass spectrometer using a Harvard syringe pump at a rate of 10 μl/min. The operating parameters were as follows: capillary voltage, 3.1 kV; cone voltage, 27 V; extractor voltage, 4 V; source block temperature, 100° C. and desolvation temperature of 200° C. Spectra were acquired at the rate of 200 amu per sec over the range of 20-2,000 amu.

In vivo regulation of AR by NO-donors: Rat erythrocytes were incubated with phosphate-buffered saline (PBS) containing freshly prepared NO donors and 1 μg/ml of cycloheximide at 37° C. for 2 h under 95% oxygen and 5% CO₂ atmosphere, followed by the addition of 5 or 40 mM glucose to the same media. Erythrocytes were incubated for another 4 h, harvested and lysed, and the protein was precipitated using 0.5 M each of barium hydroxide and zinc sulfate. The suspension was centrifuged at 10,000 g for 10 min and the clear supernatant was lyophilized using SpeedVac. The lyophilized material was dissolved and derivatized by adding 0.1 ml of the deravasil solvent. The derivatized mixture, 1 μl, was injected into a Varian Gas Chromatography System for sorbitol analysis. The amount of sorbitol present in the sample was calculated using standard reagent sorbitol measured by GC under similar conditions.

Results

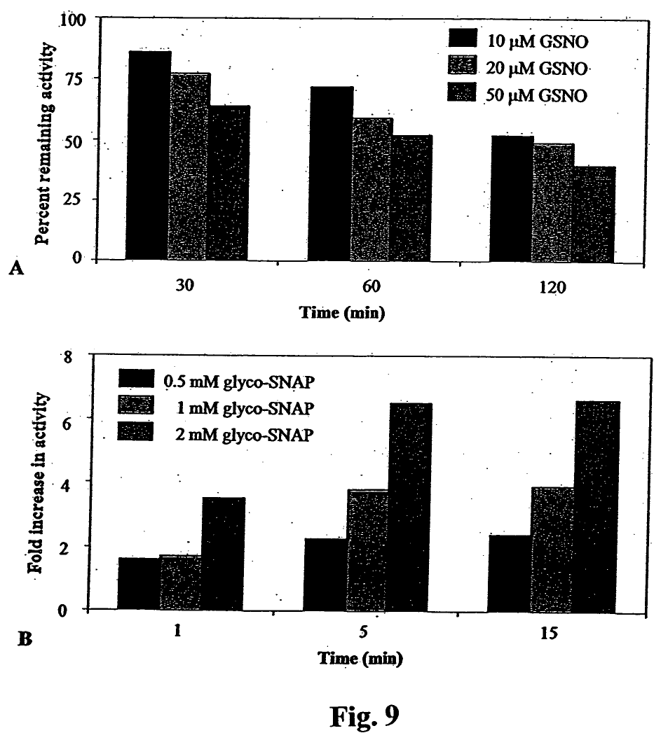

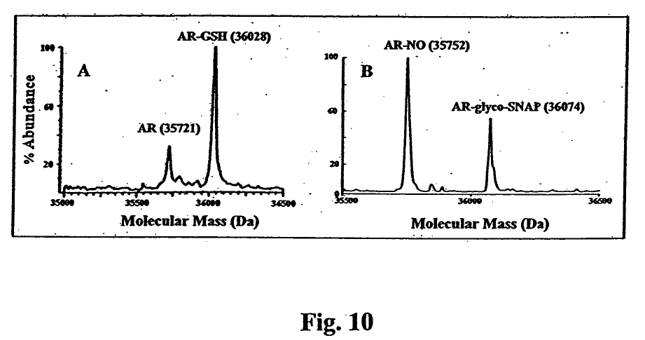

In vitro modification of AR by NO donors: Incubation of reduced recombinant AR with 10-50 μM GSNO led to a time- and concentration-dependent inactivation of the enzyme (FIG. 9A), with a second-order rate constant of 0.087±0.009 M⁻¹ min⁻¹ (data not shown). However, even upon exhaustive modification, 30-40% of the enzyme activity was retained. Significantly higher catalytic activity was retained when the enzyme was modified in the presence of NADPH, suggesting relatively low reactivity of the E-NADPH complex with GSNO. The electrospray mass spectrum of the GSNO-modified enzyme revealed a major modified species (70% of the protein) with a molecular mass of 36,028 Da (FIG. 10A), suggesting that the inactivation of AR by GSNO is due to the selective formation of a single mixed disulfide between glutathione and Cys-298 located at the NADP-(H)-binding site of the enzyme. Subsequent to the inventors observation that GSNO inhibited AR by glutathiolating Cys-298, the inventors investigated the effect of nitrosation of AR-Cys-298 by the NO donors, S-nitroso-N-acetyl penicillamine (SNAP) and N-(β-glucopyranosyl)-N-acetyl-S-nitroso-penicillamide (glyco-SNAP). Incubation of the enzyme with these NO donors resulted in a 3- to 7-fold increase in the enzyme activity (FIG. 9B). Compared to the native protein, the modified enzyme was less sensitive to inhibition by sorbinil and was not activated by sulfate anions. The ESI-MS studies revealed that the modification reaction proceeds via the formation of an adduct between glyco-SNAP and AR (FIG. 10B). Modification of AR by the non-thiol NO donor, diethylamine NONOate (DEANO) also increased enzyme activity, but resulted in the formation of a protein species with a molecular mass 30 DA more than the native protein (data not shown), consistent with the exclusive generation of AR nitrosated uniquely at a single site (AR-NO). These results demonstrate that depending upon their chemical nature, nitrosothiols can induce multiple structural modifications in AR, which could result in disparate changes in the kinetics of the enzyme protein.

In vivo regulation of AR by NO-donors: Modification of AR by NO-donors in vitro suggests that AR may also be susceptible to NO-induced modification in vivo. To determine in vivo changes in AR activity, the inventors examined the effects of several NO donors on red blood cells by monitoring changes in sorbitol formation (Table 3). For this, rat erythrocytes were incubated with 1 mM each of the NO donors—NONOate, SNAP and GSNO for 2 h and the incubation was continued for another 4 h in media containing 40 mM glucose for 4 h. As compared to cells that were incubated in the medium with no additive, cells incubated in the presence of NO donors showed decreased formation of sorbitol. Similar results were obtained with vascular smooth muscle cells (VSMC). When cultured rat VSMC were incubated with SNAP a significant decrease in the AR activity and sorbitol formation was observed (data not shown). In addition, the inventors discovered that the inactivation of AR activity was associated with S-glutathiolation of the enzyme. Inhibition of AR activity was also observed when the rat aorta was incubated with nitric oxide synthase (NOS) substrate, L-arginine and was inhibited when NOS inhibitor, L-NAME was added to the incubation medium. These results further suggest that in vivo, NO can regulate the AR activity.

TABLE 3
Nitric oxide donors prevent sorbitol formation rat erytbrocytes

| | Sorbitol | Inhibition |
|---|---|---|
| NO-Donor | (nmoles/ml RBC) | (%) |
| None | 38.33 ± 2.6 | 0 |
| SNAP | 5.17 ± 2.6** | 86.5 ± 5.0 |
| GSNO | 11.40 ± 2.3** | 70.2 ± 2.6 |
| GSNO-Ester | 10.48 ± 1.7** | 72.6 ± 3.7 |
| SIN-1 | 9.24 ± 2.5** | 75.9 ± 4.1 |
| NONOate | 10.51 ± 27** | 72.6 ± 4.6 |

Erythrocytes were isolated from normal rats and were incubated with 40 mM glucose with or without the indicated NO-Donors (1 mM) for 6 h as described under “Materials and Methods”. The sorbitol content was determined by gas chromatography. The data are mean ± SE (n = 6). Percent inhibition was calculated using the sorbitol concentration of the erythrocytes determined without NO donor.
**p < 0.001 as compared to without NO donor.

### Example 4

### Role of Aldose Reductase in TNF-α Induced Apoptosis of Vascular Endothelial Cells

Materials and Methods

Materials: Phosphate-buffered-saline (PBS), penicillin/streptomycin solution, trypsin and fetal bovine serum were purchased from GIBCO BRL Life Technologies (Grand Island, N.Y.). Consensus oligonucleotides for NF-κB (5′-AGTTGAGGGGACTTTCCCAGGC-3′) was obtained from Promega corp. Sorbinil and tolrestat were gifts from Pfizer and Ayerest, respectively. Reagents used in the electrophoretic mobility shift assay (EMSA) and Western blot analyses were obtained from Sigma. All other reagents used were of analytical grade.

Cell culture conditions: Human vascular endothelial cells (VEC) were obtained from ATCC and were maintained and grown confluent in Ham's F12K medium supplemented with 2 mM L-glutamate, 0.1 mg/ml heparin and 0.05 mg/ml endothelial growth supplement (ECGS) and 10% fetal bovine serum at 37° C. in a humidified atmosphere of 5% CO₂.

Cytotoxicity assays: The cells were grown to confluency in the indicated media and were harvested by trypsinization and were platted either 5000 cells/well in a 96 well plate. Cells were grown 24 h and at 60 to 80% confluency their growth was arrested for 24 h by replacing fresh media containing 0.1% FBS and prior to the treatment with TNF-α or aldose reductase inhibitor (ARI). Twenty-four hours after substitution of medium, cells were treated with either TNF-α (2 nM) alone or ARI (10 μM) alone or both the experimental agents for another 24 h. The rate of cell death was determined using thymidine incorporation.

Thymidine-incorporation: [³H]-thymidine (10 μCi/ml) was added to the cells 6 hr before the end of the incubation periods. Cells were harvested on Millipore multiscreen system 96-well filtration plates and were washed with PBS using multiscreen separation systems vacuum manifold. Filters were air-dried and were counted on beta counter.

Apoptosis: Apoptosis was evaluated by using “Cell Death detection ELISA” kit (Roche inc.) that measures cytoplasmic DNA-histone complexes, generated during apoptotic DNA fragmentation, and cell death detection was performed according to the manufacture's instructions and monitored spectrometrically at 405 nm.

Caspase-3 activity: The activity of caspase-3 was measured by using the specific caspase-3 substrate Z-DEVD-AFC, (CBZ-Asp-Glu-Val-Asp-AFC) which was incubated with cell lysate and the fluorescence (ex 400 nm, em 505 nm) released by the cleavage of substrate was measured by using fluorescence 96-well plate reader.

Electrophoretic mobility gel shift assays (EMSA) for NF-κB: The VEC were pretreated with various concentrations of ARI for 24 h and then TNF-α (100 pM) was added and incubated for 1 h at 37° C. The total cell cytosolic as well as nuclear extracts were prepared as described by Chaturvedi et al. (2000) [M. Chaturvedi, A. Mukhopadhyay, and B. B. Aggarwal, Assay for redox-sensitive transcription factors. Methods Enzymol. 319 (2000) 585-602.]. Consensus oligonucleotides for NF-κB transcription factor was 5′-end labeled using T4 polynucleotide kinase. The EMSA were performed as described by Chaturvedi et al (2000). Briefly, nuclear extracts prepared from various control and treated cells were incubated with respective labeled oligonucleotides for NF-κB or API for 15 min at 37° C., and the DNA-protein complex formed was resolved in 6.5% native polyacrylamide gels. After the electrophoresis the gels were dried by using a vacuum gel dryer and were autoradiographed on kodak X-ray films.

Western blot analysis for ICAM-1: The expression of ICAM-1 was determined by immunoblot analysis using specific antibodies against ICAM-1. VEC were either untreated or pretreated with ARI for 24 hr and then were treated with 100 pM of TNF-α. Equal amount of cytoplasmic extracts were subjected to 10% SDS-PAGE. After electrophoresis, the proteins were electrotransferred to nitrocellulose filters probed with rabbit polyclonal antibodies against ICAM-1, and were detected by enhanced chemiluminescence (Amersham Pharmacia Biotech, NJ).

Results

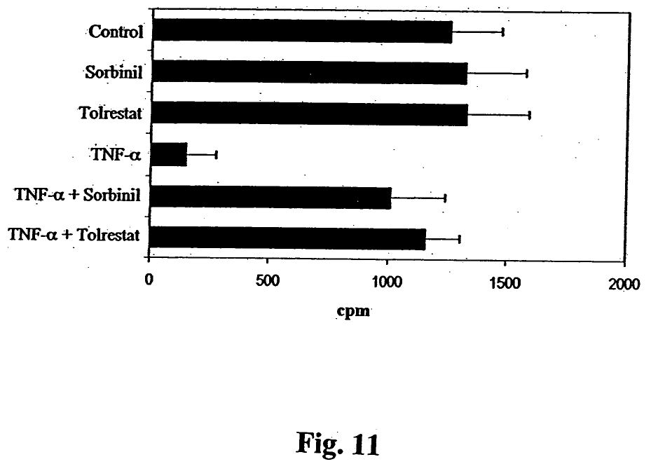

Attenuation of TNF-α induced VEC apoptosis by ARI: In the first series of experiments the inventors examined TNF-α-induced changes in VEC growth. As shown in the FIG. 11, treatment of VEC with 10 nM TNF-α for 24 h prevented VEC growth as determined by the thymidine incorporation. This effect was attenuated by two structurally distinct ARI, sorbinil or tolrestat (10 μM) added to the incubation media under identical conditions. Both sorbinil and tolrestat themselves did not cause affect VEC growth. These results show that two structurally different inhibitors of AR can prevent changes in VEC growth caused by TNF-α, suggesting the involvement of AR in the signal transduction pathway of TNF-α.

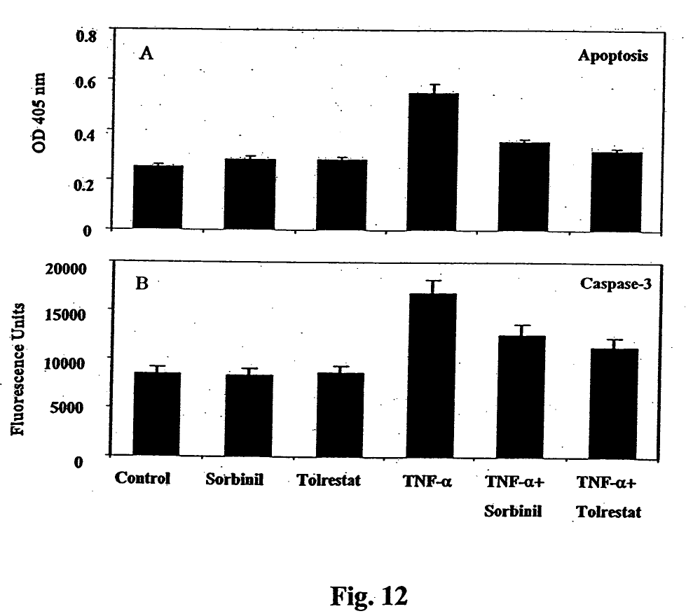

To determine whether TNF-α-mediated growth arrest was due to apoptosis, the inventors measured nucleosomal degradation as well as caspase-3 activation under identical conditions used in the above experiments. The results shown in FIG. 12A and FIG. 12B demonstrate that treatment of VEC with TNF-α caused caspase-3 activation and nucleosomal degradation. Pretreatment of VEC with sorbinil and tolrestat attenuated these changes. At the same time, ARI themselves did not result in caspase-3 activation or apoptosis, suggesting the inhibition of AR, in the absence of TNF-α stimulation does not induce cell death.

Inhibition of AR Attenuates TNF-α-Induced NF-κB Activation

For these experiments, growth-arrested VEC were preincubated for 24 h with 10 μM of tolrestat followed by the treatment with TNF-α (0.1 nM) for 60 min at 37° C., followed by the measurement of NF-κB activity by EMSA. Pretreatment with tolrestat led to an almost 60% inhibition of TNF-α-induced NF-B activation, suggesting that tolrestat is a potent inhibitor NF-κB activation. To show that tolrestat itself does not directly inhibit NF-κB, the inventors incubated the VEC with both TNF-α and tolrestat for 30 min and 60 min and examined NF-κB activation. No significant inhibition or activation of NF-κB was observed (data not shown), suggesting that pre-incubation with tolrestat is essential for preventing NF-κB activation and that tolrestat added at the same time as TNF-α does not prevent NF-κB activation. Similar type of results was obtained when the inventors used another structurally different AR inhibitor, sorbinil (data not shown).

Inhibition of AR attenuates TNF-α induced upregulation of ICAM-1: To examine whether inhibition of AR could also attenuate the expression of TNF-α induced inflammatory genes, the inventors measured changes in ICAM-1 protein expression levels by Western blot analysis. Although in untreated VEC and in tolrestat-pretreated cells, partial ICAM-1 expression was observed, a significant increase in the expression of ICAM-1 protein was observed upon treatment with TNF-α. However, pretreatment with tolrestat attenuated TNF-α-induced upregulation of ICAM-1, suggesting that inhibition of AR interrupts transcription of TNF-α/NF-κB dependent genes.

### Example 5

### Aldose Reductase Mediates Cytotoxic Signals of Hyperglycemia and TNF-α in Human Lens Epithelial Cells

Materials and Methods

Materials: Eagle's minimal essential medium (MEM), phosphate-buffered saline (PBS), gentamycin solution, trypsin and fetal bovine serum (FBS) were purchased from GIBCO BRL Life Technologies (Grand Island, N.Y.). The nuclear dye—Hoechst 33342 was obtained from Molecular Probes. Antibodies against IκB-α and p65 were obtained from Santa Cruz Biotechnology. Phospho-IκB-α (Ser³²) antibody was purchased from New England BioLabs. The antibodies against Phospho-JNK and JNK and Phospho-p38 and p38 were obtained from Cell Signaling Inc. Sorbinil and tolrestat were obtained as gifts from Pfizer and American Home Products, respectively. Mouse anti-rabbit glyceraldehyde phosphate dehydrogenase (GAPDH) antibodies were obtained from Research Diagnostics Inc., and anti-AR polyclonal antibodies against recombinant AR were raised in rabbits. Recombinant TNF-α was a gift by Dr. B. B. Aggarwal, University of Texas, M.D. Andersen, Houston. LipofectAMINE Plus and Opti-minimal essential medium were obtained from Life Technologies, Inc. Phosphorothioate AR antisense oligonucleotide (5′-CCTGGGCGCAGTCAATGTGG-3′) and mismatched control (scrambled) oligonucleotide (5-GGTGATAGCTGACGCGGTCC-3′) were used to transfect HLEC to prevent translation of AR mRNA. Consensus oligonucleotides for NF-κB (5′-AGTTGAGGGGACTTTCCCAGGC-3′) and AP1 (5′-CGCTTGATGAGTCAGCCGGAA-3′) transcription factors were obtained from Promega Corp. FLUORSAVE™ reagent was obtained from Calbiochem Corp. Phorbol 12-myristate 13-acetate (PMA), 3-(4,5-dimethylthiazol-2-yl)-2,5-diphenyl tetrazolium bromide (MTT), and other reagents used in the EMSA and Western blot analysis were obtained from Sigma Chem. Co. All other reagents were of analytical grade.

Cell culture conditions: The human lens epithelial cell line B-3 (HLEC) obtained after infecting infant human lens epithelial cells with adenovirus 12-SV40 was kindly provided by Dr. Usha P. Andley, Washington University School of Medicine, St. Louis, Mo. The cells were cultured in minimal essential media (MEM) with 20% fetal bovine serum at 37° C. in a 5% CO₂ humidified atmosphere. The cells at the 20-27 passages were used for this study.

Cytotoxicity assays: For investigating the cytotoxic effects of TNF-α and high glucose on HLEC, The cells were grown to confluency in MEM, harvested by trypsinization, and plated at a density of 5000 cells/well in a 96 well plate. The cells were grown for 12 to 24 h in the indicated media until they were 60 to 80% confluent. The cells were growth-arrested for 24 h by replacing fresh media containing 0.5% FBS and 50 μg/ml of gentamycin. The low serum levels were maintained during growth arrest to prevent slow apoptosis that accompanies complete serum deprivation. After 24 h, indicated concentrations of TNF-α or glucose without or with AR inhibitors were added to the media at the same time and the cells were incubated for another 24 h. In each dish, the number of cells was counted; cell viability was determined by the MTT assay and cell growth was estimated by thymidine incorporation. Apoptosis was determined by using Roche's cell death ELISA kit, nuclear staining with Hoechst 33342 and caspase-3 activation.

Cell count: The loss of membrane integrity, indicated by the inability of the cells to exclude trypan-blue, was used as a measure of cell viability on a hemocytometer. Briefly, the cells were harvested by trypsinization, washed with PBS and mixed with an equal amount of trypan-blue dye. The percentile of the cell population excluding trypan-blue was calculated. Four individual measurements were used for each treatment.

MTT assay: The MTT assay was used as an additional index of cell viability. After the indicated treatments, 10 μl of 5 mg/ml MTT were added to each well of the 96 well-plate and incubated at 37° C. for 2 h. The formazan granules obtained were dissolved in 100% DMSO and absorbance at 562 nm was detected using 96-well multiscanner ELISA autoreader.

Thymidine-incorporation: [³H]-thymidine (10 μCi/ml) was added to the cells 6 h before the end of incubation. Cells were harvested using Millipore multiscreen system 96-well filtration plates and were washed with PBS on a multiscreen separation system vacuum manifold. Filters were air-dried and counted on a beta scintillation counter.

Apoptosis: Apoptosis was evaluated by using “Cell Death detection ELISA” kit (Roche Inc), which measures cytoplasmic DNA-histone complexes generated during apoptotic DNA fragmentation. Cell death detection was performed according to manufacturer's instructions and monitored spectrophotometrically at 405 nm.

Nuclear staining with Hoechst 33342: After the indicated treatments, the HLEC were washed with cold PBS and incubated with 5 μg/ml of Hoechst 33342, a DNA-binding fluorescent dye, for 30 min at 4° C. The cells were examined under a fluorescent microscope (ECLIPSE E800, Nikon, Tokyo, Japan) using an excitation wavelength of 540 nm. Cells with fragmented and/or condensed nuclei were classified as apoptotic cells.

Caspase-3 activity: Caspase-3 activity was measured with the specific caspase-3 substrate Z-DEVD-AFC (CBZ-Asp-Glu-Val-Asp-AFC). The substrate was incubated with cell lysate and the product formed by the cleavage of substrate was quantified on a fluorescence 96-well plate reader using an excitation wavelength of 400 nm and emission at 505 nm.

TNF-α and High Glucose Induced Changes in Transcription Factors:

Immunostaining of HLEC cells with p65 antibodies: The cells preincubated without or with AR inhibitors for 24 h were exposed to glucose (50 mM, 2 h) or TNF-α (0.1 nM, 1 h) before immunostaining. The cells were fixed in 100% ice-cold acetone for 5 min, washed with PBS and blocked with 10% goat serum in PBS for 30 minutes. Anti-p65 antibodies were diluted 1:500 in 10% goat serum and the cells were incubated with the diluted antibodies overnight at 4° C. Following washing with PBS, the cells were incubated with respective Alexa-488 secondary antibodies in 10% goat serum for 1 h at room temperature in the dark. The cells were washed with PBS, mounted on slides and a drop of FLUORSAVE™ reagent was added. The extent of fluorescence staining was examined under a Nikon Eclipse E800 epifluorescence microscope equipped with digital camera interfaced to a computer.

Electrophoretic mobility gel shift assays (EMSA) for NF-κB and AP1: The cells were pretreated with various concentrations of AR inhibitors for 24 h and then with TNF-α (0.1 nM) for 1 h or high glucose (50 mM) for 4 h at 37° C. The cytosolic as well as nuclear extracts were prepared as described by Chaturvedi et al. (2000). Consensus oligonucleotides for NF-κB and AP1 transcription factors were 5′-end labeled using T4 polynucleotide kinase. The EMSA were performed as described by Chaturvedi et al (2000). Briefly, nuclear extracts prepared from control and treated cells were incubated with labeled oligonucleotides for NF-κB or AP1 for 15 min at 37° C., and the DNA-protein complex formed was resolved on 6.5% native polyacrylamide gels. Specificity of binding was examined by competition with an excess of unlabeled oligonucleotide. Supershift assays were also performed to determine the specificity of NF-κB binding to its specific consensus sequence by using specific antibodies to p65. After electrophoresis, the gels were dried by using a vacuum gel dryer and autoradiographed on Kodak X-ray films. The radiolabeled bands were quantified using Alpha Imager 2000 Scanning Densitometer with ALPHAEASE™ equipped with Version 3.3b software.

Western blot analysis: To determine the IκB-A phosphorylation and degradation, JNK and p38 phosphorylation, and AR expression, Western blot analyses were carried out using antibodies against IκB-α, phospho-IκB, JNK, phospho-JNK, p38, phosphop-p38 and AR. Equal amount of cytoplasmic extracts were subjected to 10% SDS-PAGE. After electrophoresis, the proteins were electrotransferred to nitrocellulose filters, probed with different antibodies and the antigen-antibody complex was detected by enhanced chemiluminescence (Amersham Pharmacia Biotech, NJ).

Measurement of Protein Kinase C (PKC) activity: To measure PKC activity, the cells were washed twice with an ice-cold PBS, and sonicated with three 10 s bursts in 1 ml of the extraction buffer (25 mM Tris-HCl, pH 7.5 containing 0.5 mM EDTA, 0.5 mM EGTA, 0.05% Triton X-100, 10 mM 2-mercaptoethanol, 1 μg/ml leupeptin, 1 μg/ml aprotinin and 0.5 mM phenylmethylsulfonyl fluoride). The homogenates were centrifuged at 100,000 g for 60 min at 4° C. in a Beckman ultracentrifuge. The pellets containing the membrane fraction were solublized by suspending in the assay buffer containing 1% Triton X-100 and stirring at 4° C. for 1 h. PKC activity was measured using the Promega Signa TECT PKC assay system. Aliquots of the reaction (25 mM Tris-HCl pH 7.5, 1.6 mg/ml phosphatidylserine, 0.16 mg/ml diacylglyceral, and 50 mM MgCl₂) were mixed with [γ-³²P] ATP (3,000 Ci/mmol, 10 μCi/μl) and incubated at 30° C. for 10 min. To stop the reaction, 7.5 M guanidine hydrochloride was added and the phosphorylated peptide was separated on binding paper. After the paper was washed, the extent of phosphorylation was detected by measuring radioactivity. The incorporation of radioactivity was linear for 15 min, and the PKC activity was determined by subtracting the initial rate of protein kinase activity (in the absence of activators) from the rate of protein kinase activity in the presence of phosphatidylserine and diacylglycerol.

Transfection with antisense oligonucleotides: Cells grown to 60-70% confluency in MEM containing 20% FBS were washed with opti-minimal essential medium four times, 60 min before transfection. The cells were incubated with 1 μM AR antisense or scrambled oligonucleotides using LipofectAMINE Plus (15 μg/ml) as the transfection reagent as suggested by the supplier. After 12 h, the medium was replaced with fresh MEM (containing 20% FBS) for another 24 h followed by 24 h of incubation in serum free-MEM (0.5% FBS) before stimulation by high glucose or TNF-α. Changes in the expression of AR were estimated by Western blot analysis using anti-AR antibodies and by measuring the AR activity in the total cell lysate. For investigating the effect of AR ablation on TNF-α and high glucose-induced apoptosis, the cells were incubated with TNF-α (2 nM) or high glucose (50 nM) for 24 h and to determine the PKC activity the cells were incubated with TNF-α (2 nM) or high glucose (50 mM) for 4 h.

Results

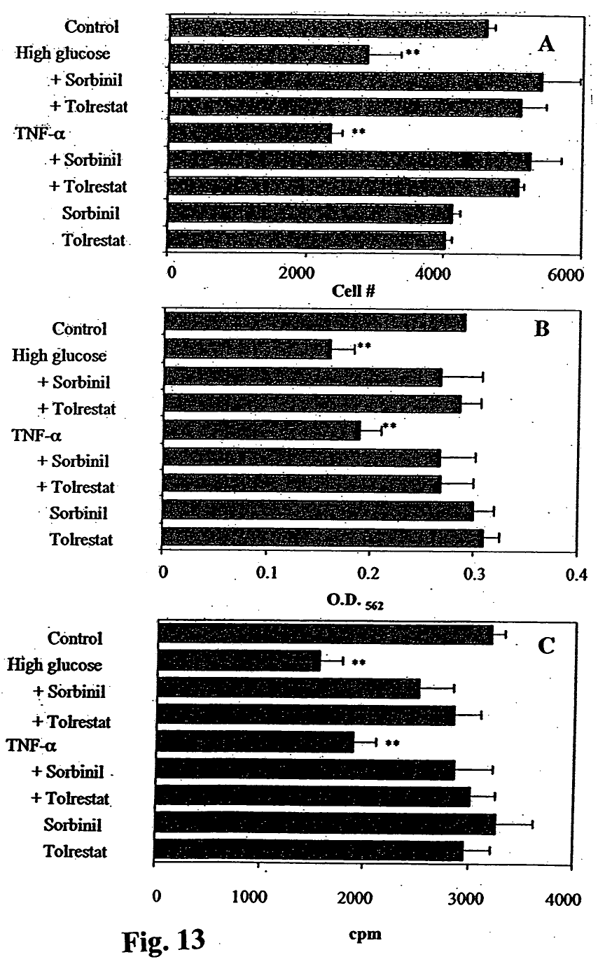

Inhibition of AR prevents TNF-α and high glucose-induced cell death: Treatment of growth-arrested HLEC with either TNF-α (2 nM) or high glucose for 24 h induced cell death as assessed by a decrease in the number of cells in the dish, MTT assay and [³H]-thymidine incorporation (FIG. 13A, FIG. 13B and FIG. 13C). The effects of high glucose and TNF-α were prevented when the cells were pretreated with AR inhibitors—tolrestat or sorbinil. Neither of the AR inhibitors induced cell death by themselves nor did they affected cell proliferation in serum-free conditions. The inhibition of the cytotoxic effects of high glucose and TNF-α by these two structurally unrelated AR inhibitors suggests that AR activity may be essential for induction of cell death under these conditions.

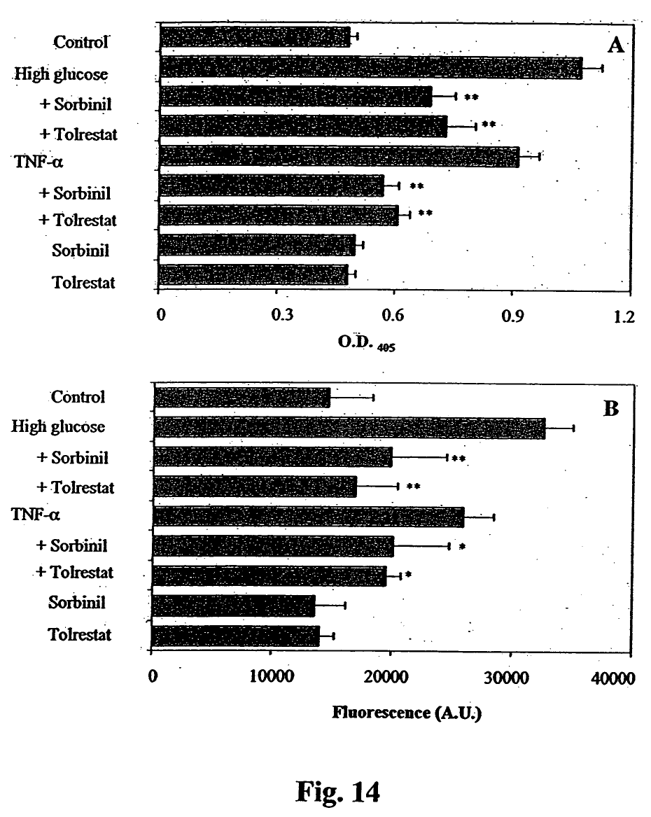

To examine the nature of cell death, the inventors measured caspase-3 activation as well as free histones released upon nucleosomal degradation. Both these indices are hallmarks of apoptotic cell death (Earnshaw et al. (1999) and Saraste et al. (2000)). As shown in FIG. 14 TNF-α as well as high glucose caused activation of caspase-3 and resulted in the degradation of nucleosomal histones. Preincubating the cells with either sorbinil or tolrestat prevented these changes. Under similar conditions, neither sorbinil nor tolrestat caused caspase-3 activation or apoptosis. To ensure accuracy of our measurements, the inventors used Hoechst 33342 staining, which can detect apoptotic cells with morphological changes leading to nuclear fragmentation (Lizard et al. (1999). Cells treated with high glucose or TNF-α displayed nuclear fragmentation and condensation, whereas, preincubation with tolrestat prevented the cells from undergoing apoptosis induced by either glucose or TNF-α.

Inhibition of AR prevents NF-κB activation: To identify changes in intracellular signaling caused by inhibiting AR, the inventors determined the activation of NF-κB by high glucose and TNF-α. Activation of this transcription factor has been shown to be a critical determinant of cell death or survival in several types of cells (Karin et al. (1999) and Tak et al. (2001)). For this, the HLEC were grown to confluency and pre-incubated for 24 h with different concentrations of sorbinil ranging from 5 to 100 μM, and then stimulated with either 0.1 nM TNF-α for 60 min or with 50 mM glucose for 2 h at 37° C. At the end of the incubation period, the cells were harvested and lysed and their nuclear extracts were prepared. The NF-κB activity was determined by EMSA as described under Materials and Methods. Pre-incubation with sorbinil caused a dose-dependent inhibition of NF-κB activation. The inhibitory effects of sorbinil were evident at 10 μM. At a concentration of 20 μM, sorbinil induced a 60% inhibition of NF-κB binding to its cognate DNA sequence. Sorbinil by itself did not affect the NF-κB activity at a concentration of 10 μM, however, at higher concentrations (20 to 100 μM) NF-κB activity was slightly inhibited. This may be a reflection of the inhibitory effect of sorbinil on basal NF-κB activation by residual growth factors and mitogens present in 0.5% serum used to maintain the serum-starved cells.

In the next series of experiments, the inventors examined the time course of sorbinil inhibition. For this, quiescent HLEC were pre-incubated with 3, 6, 12, 24, and 48 h with 10 or 20 μM sorbinil prior to 60 min exposure to TNF-α or 2 h exposure to glucose, and the NF-κB binding activity was determined as before. The inhibitory effects of sorbinil were evident after 12 h of pre-incubation, and maximal inhibition was observed in cells that were pre-incubated with sorbinil for 24 h. No additional inhibition was observed when the pre-incubation period was increased to 48 h. To determine if sorbinil would acutely inhibit TNF-α or high glucose-initiated signaling, the HLEC were incubated with TNF-α+sorbinil or glucose+sorbinil for 60 min and NF-κB activation was measured. Under these conditions, sorbinil did not significantly inhibit NF-κB activity, indicating that pre-incubation with sorbinil is essential for inhibiting NF-κB and that sorbinil does not directly interfere with NF-κB activation once the signaling cascade is initiated by either TNF-α or high glucose. Furthermore, to ascertain that the gel-retarded band visualized by EMSA in TNF-α or glucose-treated cells was indeed due to NF-κB, the inventors incubated the nuclear extract from glucose-treated or TNF-α-activated cells with anti-p65 antibodies before EMSA.

Inhibition of AR prevents nuclear translocation of the p50/p65 dimer: In unstimulated cells, the NF-κB protein is located primarily in the cytoplasm as a heterotrimer of p50, p65 and the inhibitory subunit of NF-κB (IκB-α). Upon stimulation, IκB-α undergoes phosphorylation, ubiquitination, and degradation thereby exposing the active dimer of p50/p65, which then translocates to the nucleus, and initiates the transcription of several inflammatory response genes that cause cell growth or apoptosis (Karin et al. (1999) and Tak et al. (2001). To examine which component(s) of this signaling mechanism is affected by inhibiting AR, the inventors measured the nuclear translocation of NF-κB and the phosphorylation and degradation of IκB-α. Most of the inactive form of NF-κB was present in the cytosol of unstimulated cells. Incubation with either high glucose or TNF-α led to sharp localization of fluorescence, which corresponded to the intracellular staining of the Hoeshst nuclear dye, indicating that both TNF-α and high glucose induce nuclear localization of p65. Incubation of these cells with tolrestat alone did not affect the cellular localization of p65 as evident from the diffuse staining that was comparable to that observed in untreated or unstimulated cells. However, when the tolrestat-pretreated cells were stimulated with either TNF-α or high glucose, no nuclear staining was observed and these cells continued to show diffuse perinuclear staining. This results suggest that inhibition of AR prevents high glucose or TNF-α induced nuclear translocation of p65.

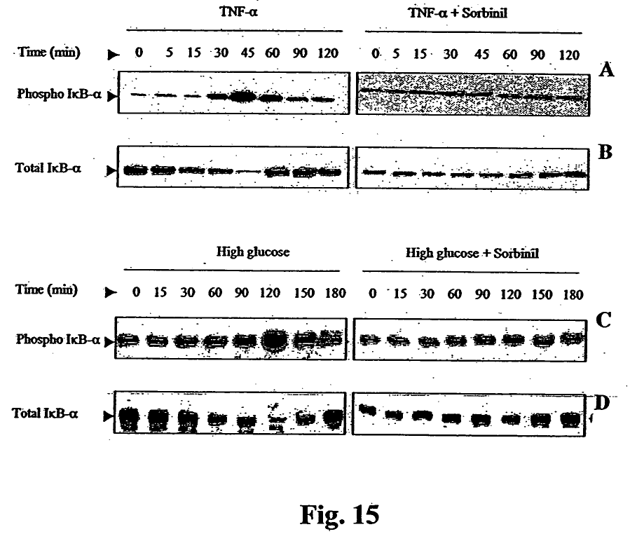

Inhibition of AR prevents degradation of IκB-α and nuclear translocation of p50/p65: The nuclear translocation of NF-κB is preceded by phosphorylation and proteolytic degradation of Iκ-Bα (Karin et al. (1999) and Tak et al. (2001). Hence, to determine whether inhibition of AR prevents events upstream to the nuclear translocation of NF-κB, the inventors examined changes in Iκ-Bα and phospho-Iκ-Bα on Western blots developed with antibodies specific to these proteins. In untreated cells, partial Iκ-Bα phoshophorylation was observed within 15 min of stimulation with TNF-α and maximal phosphorylation was evident at 45 min, after which a progressive decrease in the immunoreactive band was observed (FIG. 15A). Parallel blots developed with anti-Iκ-Bα showed transient decrease in the Iκ-Bα abundance, which was maximal at 45 min and returned to control levels within 60 to 90 min of stimulation. These observations show that stimulation with TNF-α leads to rapid phosphorylation and degradation of Iκ-Bα followed by complete resynthesis in 60 min. This sequence of events was dramatically affected by inhibiting AR. In sorbinil-treated cells, little Iκ-Bα phosphorylation was observed upon stimulation with TNF-α, and there was no change in the cellular abundance of the Iκ-α protein. A similar sequence of events, albeit with a delayed time course, was observed in HLEC cultured in high glucose. In this case, maximal phosphorylation and degradation of Iκ-Bα was observed after 120 min of stimulation, however, pretreatment with sorbinil prevented high glucose-induced Iκ-Bα phosphorylation (FIG. 15C) and degradation (FIG. 15D). Together, these results show that inhibition of AR prevents TNF-α as well as high glucose-induced phopshorylation and proteolytic degradation of Iκ-Bα.

Attenuation of PKC activation: Both TNF-α and high glucose are known to activate the PKC family of protein kinases by first activating phospholipases (Brownlee (2001), Nishikawa et al. (2000) and Terry et al. (1999). In several cell types, PKC activation is essential for stimulating downstream signaling events leading to the Iκ-Bα phosphorylation and nuclear translocation of the p65/p50 dimer (Lallena et al. (1999) and Trushin et al. (1999). The inventors, examined whether inhibition of AR would prevent NF-κB activation by phorbol ester (PMA), which bypasses the upstream signaling and directly stimulates PKC and downstream signaling. Although stimulation with PMA resulted in marked stimulation of NF-κB activity, neither sorbinil nor tolrestat prevented the PMA-induced NF-κB activation. These observations suggest that the locus of inhibition by these drugs is upstream of PKC and if PKC is directly activated, inhibition of AR does not abolish downstream signaling.

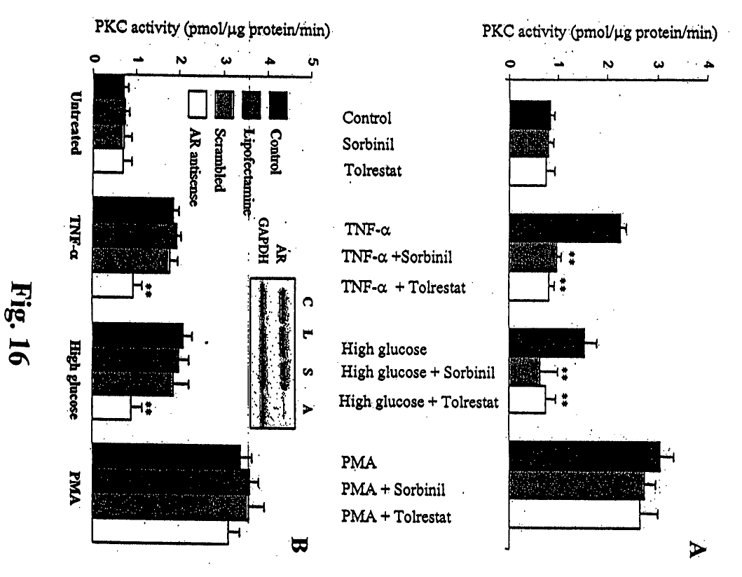

To elucidate further the effects of AR inhibitors, the inventors directly measured PKC activity in high glucose and TNF-α stimulated cells. As shown in FIG. 16, sorbinil and tolrestat by themselves did not activate or inhibit basal PKC activity. Stimulation with TNF-α or high glucose however, led to a significant increase in the membrane-bound PKC activity. The PKC activity was also dramatically increased in these cells by PMA stimulation. Pretreatment with either sorbinil or tolrestat prevented PKC activation by the increase in PKC activity in TNF-α or high glucose. Activation of cytosolic PKC was not affected by AR inhibitors (data not shown). However, the AR inhibitors did not prevent PMA-induced activation of PKC. Collectively, these results suggest that inhibition of AR does not directly affect PKC activity but prevents PKC activation by interrupting upstream signaling events, and that the pathways downstream to PKC are insensitive to AR.

Attenuation of JNK, p38 MAPK and AP1: In addition to PKC, high glucose and TNF-α also activated other kinases particularly JNK and p38, which have been shown to be critical mediators of cell growth and apoptosis, and could represent signaling events upstream or parallel to PKC (Purves et al. (2001) and Ryden et al. (2002)). The inventors, therefore, examined whether, similar to the effects observed with PKC, inhibition of AR would also prevent the activation of these MAP kinases. The phosphorylated forms of JNK and p38 MAPK were markedly enhanced in HLEC stimulated with either high glucose or TNF-α. There was no change in the expression of total JNK and p38 MAPK. Pre-incubation with sorbinil significantly attenuated the phosphorylation of JNK and p38 stimulated by TNF-α and high glucose without affecting the total cellular abundance of JNK and p38. AP1, a transcription factor, downstream to JNK and p38 (Lee et al. (2000)) was also activated by high glucose and TNF-α as determined by EMSA, and the activation was attenuated by AR inhibitors. The activation of redox-insensitive transcription factors, SP1 and OCT1 by high glucose or TNF-α was, however, not inhibited by AR-inhibitors.

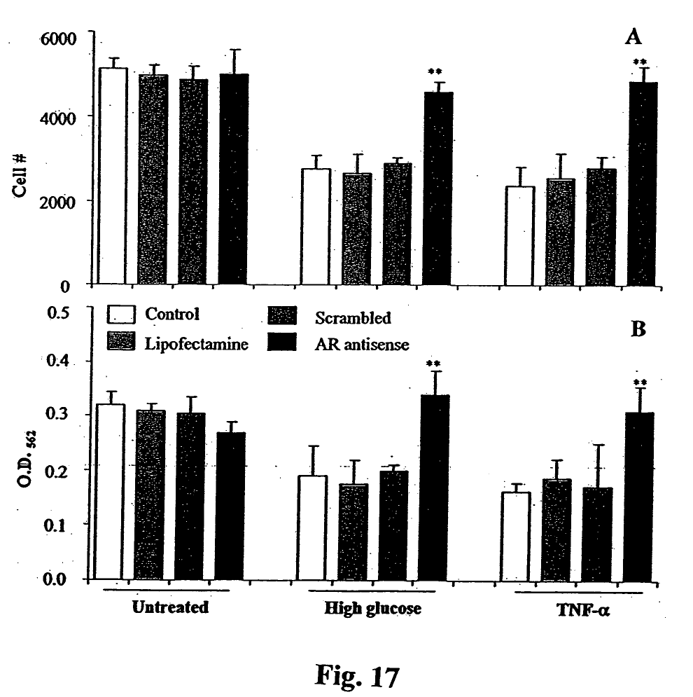

Antisense ablation of AR: Although sorbinil and tolrestat are considered relatively specific inhibitors of aldose reductase (Kinoshita (1990), Bhatnagar et al. (1992) and Yabe-Nishimura (1998)), their non-specificity cannot be rigorously excluded. The inventors therefore, examined the cellular consequences of ablating the AR message. Exposing HLEC to the antisense oligonucleotides inhibited AR expression by more than 90% as compared to scrambled oligonucleotide transfected cells (FIG. 16, inset). Antisense inhibition of AR was accompanied by a decrease in the membrane bound PKC activity in the TNF-α and glucose-treated cells. At the same time, the ablation of AR did not prevent PMA-induced activation of PKC (FIG. 16B). Interestingly, along with preventing the high glucose and TNF-α-induced PKC activation, AR ablation also prevented increased apoptosis by these agents (FIG. 17).

Inhibition of AR attenuates high glucose and TNF-α-induced apoptosis in HLEC: Incubation of the serum-starved transformed human lens epithelial cells—B3 (HLEC) with high glucose (50 mM) or TNF-α to for 24 h decreased cell growth, viability, and DNA synthesis ([³H]-thymidine incorporation) and increased caspase-3 activity, nuclear fragmentation and degradation of nucleosomal histones (measured using Roche's Cell Death ELISA kit); consistent with increased apoptosis. Pre-incubation of these cells with two structurally-unrelated AR inhibitors, i.e., sorbinil and tolrestat (10 μM each), attenuated high glucose or TNF-α-induced apoptosis, suggesting that AR may be an essential mediator of cell death-induced by high glucose or TNF-α.

Inhibition of AR abrogates high glucose and TNF-α-induced activation of NF-κB in HLEC: The transcription factor NF-κB regulates the expression of genes involved in cell growth, differentiation, inflammation, and apoptosis and is activated by oxidants, cytokines and growth factors. Therefore, the inventors examined whether the pro-apoptotic role of AR relates to NF-κB activation. Incubation of serum-starved HLEC with high glucose (50 mM) for 4 h or TNF-α for 1 h resulted in significant activation of NF-κB as measured by electrophoretic mobility gel shift assay (EMSA). Preincubation with sorbinil caused a dose-dependent inhibition of NF-κB activated by either TNF-α or high glucose. However, 10 μM sorbinil caused >60% inhibition of NF-κB activity stimulated by high glucose, whereas 20 μM sorbinil was required to cause the same extent of inhibition of NF-κB activated by TNF-α; suggesting a greater AR-dependence of high glucose signaling. Preincubation with AR-inhibitor for at least 12 h was required for inhibiting NF-κB-induction by either TNF-α or high glucose, indicating that sorbinil by itself does not directly react with components of NF-κB signaling, but that inhibition of AR prevents metabolic changes permissive of NF-κB activation.

Inhibition of AR attenuates high glucose and TNF-α-induced NF-κB translocation, IκB-α phosphorylation, and degradation: To further elucidate the involvement of AR, the inventors examined events upstream of NF-κB activation. In unstimulated cells, NF-κB is present as a heteromeric form of p65, p50 and inhibitory partner IκB, which gets phosphorylated, ubiquitinated, and degraded, leaving active NF-κB dimer of p65 and p50 to translocate into the nucleus. Incubation of serum-starved HLEC with high glucose or TNF-α caused translocation and accumulation of active NF-κB in the nuclear region. However, preincubation of serum-starved HLEC B-3 with AR inhibitors prevented the nuclear migration of NF-κB. Both high glucose and TNF-α-induced phosphorylation of IκB-α within 120 and 45 min of exposure, respectively. This was followed by degradation and rapid resynthesis of IκB-α. Preincubation of the cells with sorbinil (10 or 20 μM) attenuated glucose and TNF-α-induced IκB-α phosphorylation and degradation, indicating that inhibition of AR prevents events upstream to the activation sequelae of IκB-α.

Involvement of protein kinase C (PKC) in the activation of NF-κB in HLEC induced by high glucose and TNF-α Serum-kinases including proteins kinase C (PKC) can phosphorylate IκB-α and initiate NF-κB activation. Because IκB-α phosphorylation is mediated by upstream kinases such as PKC, MAPK and IKK, the inventors measured the effect of inhibiting AR on high glucose and TNF-α-induced activation of PKC using Promega's SignaTECT PKC assay system. Incubation of the cells with high glucose (50 mM) or TNF-α (2 nM) for 4 h led to nearly a 2-fold increase in membrane-bound PKC activity (FIG. 16A), whereas preincubation with AR-inhibitors attenuated the increase in the membrane-bound PKC induced by either high glucose or TNF-α. Interestingly, inhibition of AR did not prevent the activation of PKC or NF-κB caused by stimulating the cells with 10 nM phorbol ester (PMA) for 4 h, indicating that AR probably mediates high glucose and TNF-α signals upstream of PKC.

To rule out the nonspecific effects of AR-inhibitors, the inventors transfected the HLEC with AR antisense oligonucleotides. This treatment led to a significant decrease in the AR activity and AR protein (as quantified by Western blot analysis using recombinant AR antibodies), whereas treatment with scrambled oligonucleotides had no effect. Compared with untransfected cells or cells transfected with scrambled oligonucleotides, the AR antisense-transfected cells displayed less PKC activation upon stimulation by high glucose or TNF-α. Transfection with AR antisense did not affect PKC activation by PMA (FIG. 16B). Antisense ablation of AR also attenuated apoptosis induced by high glucose and TNF-α. These observations confirm that AR plays a critical role in PKC-NF-κB signaling leading to apoptosis and that the changes observed with AR inhibitors are not due to the non-specific effects of these drugs.

Inhibition of AR specifically attenuates redox-sensitive signals: In addition to PKC, the inventors examined the effect of AR inhibition on other apoptotic signaling events such as phosphorylation of JNK, p38, and the activation of AP1, SP1, and OCT1. Incubation of HLEC with high glucose or TNF-α, induced phosphorylation of JNK and p38 but did not affect the total cellular abundance of these proteins. Preincubation of the cells with AR inhibitors attenuated high glucose and TNF-α-induced phosphorylation of JNK and p3⁸ but did not affect total JNK and p38. The high glucose and TNF-α-induced activation of transcription factor, AP1, which is downstream to JNK/p38, was also attenuated by AR-inhibitors. However, the AR inhibitors had no effect on the high glucose or TNF-α-stimulated redox-insensitive transcription factors, SP1 or OCT1, further indicating that inhibition of AR specifically affects redox-sensitive signaling events initiated by high glucose and TNF-α.

AR activity is essential for the apoptotic signaling events associated with high glucose or TNF-α stimulation. Inhibition of this enzyme prevents apoptosis as well as the activation of the PKC/NF-κB pathway. Aldose reductase represents the first and the rate-limiting step in the polyol pathway, which is a subsidiary route for glucose metabolism. Although under normal physiological conditions, the AR catalyzed transformation represents only a minor fate of glucose, under hyperglycemia, where the glucose concentration is increased, or under stress when AR is activated, reduction to sorbitol may be an important route of glucose metabolism. However, because the AR consumes NADPH and generates osmotically active polyols, increased flux of glucose via AR has been linked with oxidative and osmotic stress. In agreement with this view, inhibition of AR has been shown to prevent tissue injury and dysfunction associated with chronic exposure to high glucose or galactose or due to long-term diabetes.

The inventors discovered that exposure to high glucose or TNF-α induces cell death in HLEC with features characteristic of apoptosis. Inhibition of AR by using specific-inhibitors or antisense oligonucleotides prevented apoptosis in these cells, suggesting that AR is essential for the metabolic and signaling events that precede programmed cell death. Inhibition of AR prevented the activation of cellular kinases JNK, p38 and PKC and the activation of redox-sensitive transcription factors like NF-κB and AP1. Significantly, inhibition of AR did not prevent the activation of redox-insensitive transcription factors SP1 and OCT1 and did not prevent the direct activation of PKC by phorbol ester. AR-dependent metabolism is essential for cytokine and high glucose-mediated cell death and that inhibition of this enzyme prevents redox-sensitive events preceding the activation of PKC and NF-κB. Because oxidative stress has been suggested to be a causative factor in the development of diabetic and hyperglycemic injury, the results of this discovery may be of significance to the understanding and the treatment of diabetic complications.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the methods described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

These references are specifically incorporated by reference.

U.S. Pat Pub. 20030059944

U.S. Pat Pub. 20030105051

U.S. Pat. No. 4,682,195

U.S. Pat. No. 4,683,202

U.S. Pat. No. 5,354,855

U.S. Pat. No. 5,359,046

U.S. Pat. No. 5,359,046

U.S. Pat. No. 5,582,981

U.S. Pat. No. 5,645,897

U.S. Pat. No. 5,756,291

U.S. Pat. No. 5,780,610

U.S. Pat. No. 5,792,613

U.S. Pat. No. 5,840,867

PCT App. No. WO 03/012052

Aggarwal et al., Ann. Rheum. Dis. 59 (S1), i6-16, 2000.

Airey et al., Cochrane Database Syst Rev. 2:CD002182, 2000.

Akaike, Free. Radic. Res. 33, 461-469, 2000.

Barany and Merrifield, The Peptides, Gross and Meienhofer, eds., Academic Press, New York, pp. 1-284 1979.

Barber et al., J. Clin. Invest. 102, 783-791, 1998.

Barisani et al., FEBS Lett. 469, 208-212, 2000.

Baynes et al., Free Radic. Biol. Med, 28:1708-1716, 2000.

Beckman et al., JAMA. 287:2570-2581, 2002.

Berk, Thromb. Haemost 82:810-817, 1999.

Berzal-Herranz et al., Genes Dev, 6(1):129-134, 1992.

Bhatnagar et al., Biochem. Med. Metab. Biol. 48, 91-121, 1992.

Bhatnagar et al., Biochem. Pharmacol. 39, 1115-1124, 1990.

Bierhaus et al., Diabetes 50, 2792-808, 2001.

Blann et al., Inflammation 22:483-491, 1998.

Blass et al., Mol. Cell. Biol. 22:182-195, 2002.

Bours et al., Toxicology 153, 27-38, 2000.

Brownlee, Nature 414:813-820, 2001.

Bucala, Exp. Phsyiol. 82:327-337, 1997.

Burg et al., Ann. Rev. Physiol. 59, 437-55, 1997.

Burg, Am. J. Physiol. 268, F983-F996, 1995.

Burgstaller et al., Curr. Opin. Drug Discov. Devel., 5(5):690-700, 2002.

Cai et al., Circ. Res. 87:840-844, 2000.

Cao et al., Cold Spring Harb Symp. Quant. Biol. 64:473-483, 1999.

Cech et al., Cell, 27(3 Pt 2):487-496, 1981.

Chandra et al., Biochemistry 36, 15801-15809, 1997.

Chaturvedi et al., Methods. Enzymol. 319, 585-602, 2000.

Chen and Okayama, Mol. Cell Biol., 7(8):2745-2752, 1987.

Chowrira et al., J. Biol. Chem., 268:19458-62, 1993.

Chowrira et al., J. Biol. Chem., 269(41):25856-25864, 1994.

Choy et al., J. Mol. Cell. Cardiol. 33:1673-1690, 2001.

Collins et al., J. Clin. Invest. 107:255-264, 2001.

Costantino et al., Med. Res. Rev. 19:3-23, 1999.

Czech et al., J. Biol. Chem. 274,1865-1868, 1999.

Davis, Cell 103:239-252, 2000.

Dempsy et al., Am. J. Physiol. 279:L429-L438, 2000.

Denninger et al., Biochim. Biophys. Acta. 1411, 334-350, 1999.

Dixit et al., J. Biol. Chem. 275, 21587-21595, 2000.

Donohue et al., J. Biol. Chem. 269, 8604-8609, 1994.

Droge, Physiol. Rev. 82:47-95, 2002.

Dyck et al., Diabetic Neuropathy. W. B. Sauders Co. Philadelphia, 1999.

Earnshaw et al., Ann. Rev. Biochem. 68:383-424, 1999.

Elkind et al., Stroke 33:31-37, 2002.

Fechheimer et al., Proc. Natl. Acad. Sci. USA, 84:8463-8467, 1987.

Ferraris et al., J. Biol. Chem. 271:18318-18321, 1996.

Fiordaliso et al., Diabetes, 50:2363-2375, 2001.

Forster and Symons, Cell, 49(2):211-220, 1987.

Fraley et al., Proc. Natl. Acad. Sci. USA, 76:3348-3352, 1979.

Fukagawa et al., Free Radic Biol Med 31:1560-1569, 2001.

Gerlach et al., Nature 328:802-805, 1987.

Giardino et al., Diabetes 47:1114-1120, 1998.

Golovchenko et al., Circ. Res. 87, 746-752, 2000.

Gonzalez et al., Diabetes 33:196-199, 1984.

Gopal, Mol. Cell. Biol., 5:1188-1190, 1985.

Graham and Van Der Eb, Virology, 52:456-467, 1973.

Grant et al., Mol. Cell. Biol. 19, 2650-2656, 1999.

Greene et al., Eur. J. Pharmacol. 375:217-223, 1999.

Hajra et al., Proc. Natl. Acad. Sci. USA 97, 9052-9057, 2000.

Hamaoka et al., J. Biochem. 126:41-47, 1999.

Harland and Weintraub, J. Cell Biol., 101:1094-1099, 1985.

Harlow and Lane, Antibodies: A Laboratory Manual. Cold Spring Harbor Laboratory Press, Cold Spring harbor, N.Y., 553-612, 1988.

Haseloff and Gerlach, Nature, 334:585-591, 1988.

Hashimoto et al., Br. J. Pharmacol. 132:270-276, 2001.

Henry et al., Am. J. Physiol 277, F97-F104, 1999.

Henry et al., Cell Mol. Life. Sci. 55,1003-1014, 1999.

Herr et al., Blood 98:2603-2614, 2001.

Ho et al., Circulation, 101:2618-2624, 2000.

Ho et al., Proc. Soc. Exp. Biol. Med. 222, 205-213, 1999.

Hoshi et al., J. Biol. Chem. 275, 883-889, 2000.

Hotta, Nagoya J. Med. Sci. 60, 89-100, 1997.

Hsu et al., Biochem. J. 328, 593-598, 1997.

Hurst et al., Brain Res 894:181-188, 2001.

Iwata et al., J. Biol. Chem. 274:7993-8001, 1999.

Jacquin-Becker et al., GLIA. 20, 135-44, 1997.

Jeay et al., Endocrinology 142(1):147-156, 2001..

Jez et al., Biochem. J. 326, 625-636, 1997.

Jourd'heuil et al., J. Clin. Gastroenterol. 25, S61-S72, 1997.

Joyce, Nature, 338:217-244, 1989.

Kador et al., Annu. Rev. Pharmacol. Toxicol. 25, 691-714, 1985.

Karin, J. Biol. Chem. 274:27339-27342, 1999.

Kassab et al., Vasc. Med. 6, 249-255, 2001.

Kim and Cech, Proc. Natl. Acad. Sci. USA, 84:8788-8792, 1987.

King et al., Diabetes 45, S105-S108, 1996.

Kinoshita, Exp Eye Res 50:567-573, 1990.

Kirpichnikov, Trends Endocrinol. Metab. 12:225-230, 2001.

Klatt et al., Eur. J. Biochem. 267, 4928-4944, 2000.

Knickelbein et al., Am J Physiol. 270:L115-122, 1996.

Ko et al., Biochem. Biophys. Res. Comm. 270, 52-61, 2000.

Konorev et al., Free Radic Biol Med. 28:1671-1678, 2000.

Koya et al., Diabetes 47:859-866, 1998.

Kunsch et al., Circ. Res. 85:753-766, 1999.

Kuo et al., Mol Cell Biochem. 233:57-63, 2002.

Lallena et al., Mol. Cell. Biol. 19:2180-2188, 1999.

Lee et al., Mol. Pharmacol. 58:1536-1545, 2000.

Lee et al., Proc. Natl. Acad. Sci. USA. 92, 2780-2784, 1995.

Li et al., J Pathol., 190:244-254, 2000.

Lieber and Strauss, Mol. Cell. Biol., 15: 540-551, 1995.

Litherland et al., Mol. Membr. Biol. 18, 195-204, 2001.

Liu et al., Biochem. Biophys. Acta. 1120:329-336, 1992.

Liu et al., Biochim Biophys Acta. 1164, 268-272, 1993.

Lizard et al., Arterioscl. Thromb. Vase Biol. 19:1190-1200, 1999.

Majumdar et al., J. Biol. Chem. 275:21793-21796, 2000.

Mallat et al., Br. J. Pharmacol. 130:947-962, 2000.

Mallis et al., Biol. Chem. 383:649-662, 2002.

Mathias et al., Am. J. Physiol. 249:C181-C190, 1985.

Merrifield, Science, 232: 341-347, 1986.

Michel and Westhof, J. Mol. Biol., 216:585-610, 1990.

Mitchell et al., Semin. Vasc. Surg. 11:134-141, 1998.

Miyakawa et al., Proc. Natl. Acad. Sci. USA. 96, 2538-42, 1999

Moller, Trends Endo Metabol 11:212-217, 2000.

Murata et al., Chem-Bio Interact. 130-132:617-625, 2001.

Nakamura et al., Diabetes 48, 2090-2095, 1999.

Nakamura et al., Diabetologia 44:480-487, 2001.

Nakamura et al., Free Rad. Biol. Med. 29, 17-25, 2000.

Nakamura et al., J. Biol. Chem., 277:2687-2694, 2002.

NCBI webpage, 2002.

Nicolau and Sene, Biochim. Biophys. Acta, 721:185-190, 1982.

Niemann-Jonsson et al., Arterioscle. Thromb. Vasc. Biol. 21, 1909-14, 2001.

Nishikawa et al., Kidney Internt Suppl. 77:S26-S30, 2000.

Nishikawa et al., Nature 404, 787-790, 2000.

O'Connor et al., Biochem. J. 343, 487-504, 1999.

Ortiz et al., J. Invest. Med. 45:50-56, 1997.

Palukaitis et al., Virology, 99:145-151, 1979.

Pampfer et al., Diabetes 46:1214-1224, 1997.

Perriman et al., Gene, 113:157-163, 1992.

Perrotta and Been, Biochemistry, 31(1):16-21, 1992.

Ping et al., Circ. Res. 88:59-62, 2001.

Podesta et al., Am. J. Pathol. 156:1025-1032, 2000.

Potter et al., Proc. Nat. Acad. Sci. USA, 81:7161-7165, 1984.

Prody et al., Science, 231:1577-1580, 1986.

Purves et al., FASEB J. 15:2508-2514, 2001.

Rabinovitch, Diabetes Metab. Rev. 14:129-151, 1998.

Ramana et al., Biochemistry 39, 12172-12180, 2000.

Rath et al., J. Clin. Immunol. 19, 350-364, 1999.

Reaven et al., Arterioscler Thromb. Vase. Biol. 17:2250-2256, 1997.

Recchia, Heart. Fail. Rev. 7, 141-148, 2002.

Rectenwald et al., Circulation 102, 1697-1702, 2000.

Reinhold-Hurek and Shub, Nature, 357:173-176, 1992.

Remington's Pharmaceutical Sciences, 18th Ed. Mack Printing Company, 1289-1329, 1990.

Richardson and Marasco, Trends Biotechnol., 13(8):306-310, 1995.

Rippe et al., Mol. Cell Biol., 10:689-695, 1990.

Rittner et al., J. Clin. Invest. 103, 1007-1013, 1999.

Rossig et al., J. Biol. Chem. 274:6823-6826, 1999.

Ruderman et al., FASEB J. 6, 2905-2914, 1992.

Ruef et al., Arterioscler. Thromb. Vasc. Biol. 20, 1745-1752, 2000.

Ryden et al., J. Biol. Chem. 277:1085-1091, 2002.

Salomon et al., Chem. Res. Toxicol. 13:557-564, 2000.

Sambrook et al., In: Molecular cloning, Cold Spring Harbor Laboratory Press, Cold Spring Harbor, N.Y., 1989.

Saraste et al., Cardiovas. Res. 45:528-537, 2000.

Sarver et al., Science, 247:1222-1225, 1990.

Scanlon et al., Proc. Natl. Acad. Sci. USA, 88:10591-10595, 1991.

Schmidt et al., Circ. Res. 84:489-497, 1999.

Selzman et al., Circ. Res. 84, 867-875, 1999.

Seo et al., Mol. Pharmacol. 57, 709-717, 2000.

Shinmura et al., Circulation 102(Suppl II), 11-120, 2000.

Shizukuda et al., Am. J. Physiol. 282:H1625-H1634, 2002.

Shrivastava et al., Antioxid. Redox Signal 1:181-191, 1999.

Sioud et al., J. Mol. Biol., 223:831-835, 1992.

Spycher et al., FASEB J. 11, 181-188, 1997.

Srinivasan et al., Diabetes 49:1932-1938, 2000.

Srinivasan et al., J. Clin. Invest. 102:1454-1462, 1998.

Srivastava et al., Atherosclerosis 18:339-350, 2001.

Srivastava et al., Biochem J. 358, 111-118, 2001.

Srivastava et al., Biochem. Biophys. Res. Comm., 217:741-746, 1995.

Srivastava et al., Biochemistry 38, 42-54, 1999.

Srivastava et al., Biochemistry 37, 12909-12917, 1998.

Srivastava et al., Chemico-Biological Interactions, 130-132:563-571, 2001.

Srivastava et al., Free Radic. Biol. Med. 29:642-651, 2000.

Srivastava et al., Invest. Ophthalmol. Vis. Sci. 38:2300-2312, 1997.

Srivastava et al., J. Biol. Chem. 273, 10893-10900, 1998.

Srivastava et al., Proc. Natl. Acad. Sci. USA. 82, 7222-7226, 1985.

Srivastava et al., Prog. Clin. Biol. Res, 304:171-184, 1989.

Stewart and Young, Solid Phase Peptide Synthesis, 2d. ed., Pierce Chemical Co., 1984.

Symons, Annu. Rev. Biochem., 61:641-671, 1992.

Symons, Annu. Rev. Biochem., 61:641-671, 1992.

Tak et al., J. Clin. Invest. 107:7-11, 2001.

Tam et al., J. Am. Chem. Soc., 105:6442, 1983.

Terry et al., Am. J. Physiol. 276:H1493-H1501, 1999.

Thompson et al., Nature Genet., 9:444-450, 1995.

Torreilles, Front. Biosci. 6, D1161-D1172, 2001.

Traverse et al., Am. J. Physiol. Heart. Circ. Physiol. 282:H2278-H2283, 2002.

Trushin et al., J. Biol. Chem. 274:22923-22931, 1999.

Tur-Kaspa et al., Mol. Cell Biol., 6:716-718, 1986.

Uchida et al., Biochemistry 33:12487-12494, 1994.

van Goor et al., Nitric Oxide 5, 525-33, 2001.

Vander Jagt et al., Biochim. Biophys. Acta., 1249,117-126, 1995.

Vincent et al., Ann. N.Y. Acad. Sci. 959:368-383, 2002.

Wallace, et al., Nucl. Acid Res., 9:879, 1981.

Wang et al., FEBS Lett. 508, 360-364, 2001.

Wang et al., Invest. Ophthalmol. Vis. Sci. 42:194-199, 2001.

Wennmalm, Blood Press 3:279-22, 1994.

West et al., Synapse 44, 227-245, 2002.

Wu and Wu, Biochemistry, 27:887-892, 1988.

Wu and Wu, J. Biol. Chem., 262:4429-4432, 1987.

Wu, J. Clin. Lab. Anal. 7, 293-300, 1993.

Yabe-Nishimura, Pharmacol. Rev. 50, 21-33, 1998.

Yamanouchi et al., Exp. Anim. 49:267-274, 2000.

Yang et al., Circulation 102, 3046-3052, 2000.

Yang et al., Proc Natl. Acad. Sci. USA, 87:9568-9572, 1990.

Yasunari et al., Arterioscler Thromb Vasc Biol. 15:2207-2212, 1995.

Yasunari et al., Hypertension 35:1092-1098, 2000.

Yoon et al., Ann N.Y. Acad. Sci. 928:200-211, 2001.

Yuan and Altman, Science, 263:1269-1273, 1994.

Yuan et al., Proc. Natl. Acad. Sci. USA, 89:8006-8010, 1992.

Zhang et al., Biochem Mol Med 61:58-62, 1997.

What is claimed is:

1. A method of reducing the risk of diabetes complications in a patient comprising administering to the patient an effective amount of a composition comprising a nitric oxide inducer, whereby the amount of nitric oxide in the patient is increased.

2. The method of claim 1, wherein the nitric oxide inducer is nitric oxide, a nitric oxide precursor, a nitric oxide donor, or an inhibitor of a nitric oxide synthase inhibitor.

3. The method of claim 2, wherein the composition comprises a nitric oxide precursor.

4. The method of claim 3, wherein the nitric oxide precursor is L-arginine.

5. The method of claim 4, wherein the composition comprises a nitric oxide donor.

6. The method of claim 5, wherein the nitric oxide donor is a nitric oxide synthase substrate, sildenafil citrate, nitroglycerine, S-nitrosoglutathione (GSNO), GSSG, or HNE.

7. The method of claim 6, wherein the nitroglycerine is administered to the patient by applying a nitroglycerine patch to the patient.

8. The method of claim 6, wherein the nitric oxide donor is a nitric oxide synthase substrate.

9. The method of claim 8, wherein the nitric oxide synthase substrate is L-arginine.

10. The method of claim 2, wherein the composition comprises an inhibitor of a nitric oxide synthase inhibitor.

11. The method of claim 10, wherein the nitric oxide inducer inhibits at least one of the following nitric oxide synthase inhibitors: L-NAME or L-NNA.

12. The method of claim 1, wherein the composition comprises more than 1 nitric oxide inducer.

13. The method of claim 1, wherein the diabetes complication is cataractogenesis, neuropathy, nephropathy, retinopathy, vasculopathy, atherosclerosis, restenosis, artery or vein graft rejection, or wound healing.

14. The method of claim 1, further comprising identifying a patient with diabetes or a patient at risk for diabetes complications.

15. The method of claim 1, further comprising administering insulin to the patient.

16. The method of claim 1, wherein the patient is administered the composition directly, locally, topically, orally, endoscopically, intratracheally, intratumorally, intravenously, intralesionally, intramuscularly, intraperitoneally, regionally, percutaneously, or subcutaneously.

17. The method of claim 1, wherein the composition is a pharmaceutically acceptable formulation.

18. A method of reducing the risk of diabetes complications in a patient comprising modifying a cysteine at position 298 (Cys-298) of aldose reductase (AR) in the patient, wherein AR activity is reduced.

19. The method of claim 18, wherein the Cys-298 is modified by administering to the patient a nitric oxide inducer, a hydrogen peroxide inducer, lipid-peroxidation derived aldehydes, or advanced glycosylation end products.

20. The method of claim 19, wherein the Cys-298 of AR is modified by administering to the patient a nitric oxide inducer.

21. The method of claim 20, wherein the nitric oxide inducer is a nitric oxide precursor, a nitric oxide donor, or an inhibitor of nitric oxide synthase inhibitor.

22. A method for preventing inflammation in a patient comprising administering to the patient an effective amount of a composition comprising a nitric oxide inducer.

23. The method of claim 22, wherein the nitric oxide inducer is a nitric oxide precursor, a nitric oxide donor, or an inhibitor of nitric oxide synthase inhibitor.

24. The method of claim 23, wherein the composition comprises a nitric oxide precursor.

25. The method of claim 24, wherein the nitric oxide precursor is L-arginine.

26. The method of claim 25, wherein the composition comprises a nitric oxide donor.

27. The method of claim 26, wherein the nitric oxide donor is a nitric oxide synthase substrate, sildenafil citrate, or nitroglycerine

28. The method of claim 27, wherein the nitroglycerine is administered to the patient by applying a nitroglycerine patch to the patient.

29. The method of claim 27, wherein the nitric oxide donor is a nitric oxide synthase substrate.

30. The method of claim 29, wherein the nitric oxide synthase substrate is L-arginine.

31. The method of claim 23, wherein the composition comprises an inhibitor of a nitric oxide synthase inhibitor.

32. The method of claim 31, wherein the nitric oxide inducer inhibits at least one of the following nitric oxide synthase inhibitors: L-NAME and L-NNA.

33. The method of claim 22, wherein the composition comprises more than 1 nitric oxide inducer.

34. The method of claim 22, wherein the patient is administered the composition directly, locally, topically, orally, endoscopically, intratracheally, intratumorally, intravenously, intralesionally, intramuscularly, intraperitoneally, regionally, percutaneously, or subcutaneously.

35. The method of claim 22, wherein the composition is a pharmaceutically acceptable formulation.

36. A method for treating a patient with a disease or condition involving aldose reductase or at risk for the disease or condition comprising administering to the patient an effective amount of a composition comprising a nitric oxide inducer, thereby conferring a therapeutic benefit on the patient.

37. The method of claim 36, wherein the disease or condition is selected from the group consisting of diabetes, diabetes complications, toxic shock, allergy, asthma, anaphylaxis, hyperglycemia-induced atherosclerosis, cataractogenesis, neuropathy, nephropathy, retinopathy, vasculopathy, an open wound, inflammation, restenosis, artery or vein graft rejection, complications from or with wound healing, microvaculopathy, macroangiopathy, heart disease, stroke, ischemia, septicemia, ischemic damage, arteriosclerosis, iron overload, alcholic liver disease, hear failure, myocardial ischmia, vascular inflammation, and stress.

38. A method for reducing an immune response in a patient comprising administering to the patient an effective amount of a composition comprising a nitric oxide inducer.

39. The method of claim 38, wherein the patient exhibits symptoms of asthma.

40. The method of claim 38, wherein the patient exhibits symptoms of an allergic reaction.

41. The method of claim 40, wherein the patient exhibits symptoms of anaphylaxis.

42. The method of claim 38, further comprising administering to the patient adrenaline, epinephrine, or a derivative or analog thereof.

43. A method for treating a patient with an allergy comprising administering to the patient an effective amount of a composition comprising a nitric oxide inducer, whereby the patient receives a therapeutic benefit.

44. A method for treating a patient undergoing anaphylaxis comprising administering to the patient an effective amount of a composition comprising a nitric oxide inducer, wherein the patient receives a therapeutic benefit.

45. A method for relieving asthma symptoms in a patient comprising administering to the patient an effective amount of a composition comprising a nitric oxide inducer, whereby asthma symptoms in the patient are reduced.

46. A method for reducing a reaction in a patient to a toxin comprising administering to the patient an effective amount of a composition comprising a nitric oxide inducer, whereby the reaction to the toxin is reduced.

47. The method of claim 46, wherein the toxin is a bacterial toxin.

48. A method of treating hyperglycemia-induced atherosclerosis in a patient comprising administering to the patient an effective amount of a composition comprising a nitric oxide inducer, wherein a symptom of atherosclerosis is reduced.

49. A method of reducing or preventing stress-induced change in a patient comprising administering to the patient an effective amount of a composition comprising a nitric oxide inducer, wherein the stress-induced change is reduced or prevented.

50. A method of inhibiting aldose reductase in a cell comprising providing to the cell a nitric oxide inducer, wherein aldose reductase activity is inhibited.

51. The method of claim 50, wherein the cell is in a patient.

52. The method of claim 51, wherein the patient exhibits symptoms of diabetes, diabetes complications, toxic shock, allergy, asthma, anaphylaxis, hyperclycemia-induced atherosclerosis, cataractogenesis, neuropathy, nephropathy, retinopathy, vasculopathy, an open wound, or stress.

53. A method of treating or preventing cancer in a patient comprising administering an effective amount of a composition comprising a nitric oxide inducer, whereby a therapeutic benefit is conferred upon the patient.

54. The method of claim 53, further comprising administering to the patient a second-anti-cancer therapy selected from the group consisting of chemotherapy, radiotherapy, hormone therapy, and gene therapy.

55. A method of reducing aldose reductase in a cell comprising administering an inhibitor of aldose reductase, whereby the inhibitor reduces the amount of aldose reductase in the cell.

56. The method of claim 55, wherein the inhibitor comprises a nucleic acid molecule complementary to all or part of a mRNA encoding aldose reductase.

57. The method of claim 56, wherein the nucleic acid molecule is an antisense molecule.

58. The method of claim 56, wherein the molecule is a ribozyme.

59. The method of claim 56, wherein the molecule is an siRNA.

60. A method for reducing NF-κB activity in a cell comprising administering to the cell an effective amount of aldose reductase inhibitor, wherein the activity of NF-κB is reduced.

61. The method of claim 60, wherein the activity of NF-κB is reduced by reducing the amount of NF-κB capable of activating transcription.

62. The method of claim 60, wherein the cell is in a patient with diabetes, inflammation, or atherosclerosis, or at risk for diabetes, inflammation, or atherosclerosis.

63. The method of claim 1, further comprising administering to the patient an inhibitor of NF-κB.

64. A method for screening for a candidate aldose reductase inhibitor comprising:
a) contacting aldose reductase with a candidate substance; and,
b) assaying for S-glutathiolation of aldose reductase, wherein S-glutathiolation of aldose reductase identifies substance as a candidate aldose reductase inhibitor.

65. The method of claim 64, wherein the candidate substance is an NO donor.

66. The method of claim 64, further comprising assaying the activity of S-glutathiolated aldose reductase.

67. A method of reducing ICAM-1 expression in a cell comprising administering to the cell an effective amount of a composition comprising an aldose reductase inhibitor.

68. A method of inhibiting TNF-α-induced apoptosis in a cell comprising administering to the cell an effective amount of a composition comprising an aldose reductase inhibitor.

69. A method of inhibiting apoptosis of a vascular endothelial cell comprising administering to the cell an effective amount of a composition comprising an aldose reductase inhibitor.

70. The method of claim 69, wherein the cells are in a patient exhibiting symptoms of atherosclerosis, restenosis, microvaculopathy, or macroangiopathy or the patient is at risk for atherosclerosis, restenosis, microvaculopathy, or macroangiopathy.

71. A method of screening for an aldose reductase inhibitor comprising:
a) stimulating a cell with TNF-α in the presence of a candidate substance,
b) assaying for apoptosis of the cell, wherein inhibition of apoptosis identifies the cell as a candidate aldose reductase inhibitor; and,
c) determining whether the candidate aldose reductase inhibitor inhibits the activity of aldose reductase.
